(19) United States
(12) Patent Application Publication
(10) Pub. No.: US 2007/0292936 A1
(43) Pub. Date: Dec. 20, 2007

(54) BINDING POLYPEPTIDES WITH OPTIMIZED SCAFFOLDS

(75) Inventors: Pierre A. Barthelemy, Burlingame, CA (US); Sachdev S. Sidhu, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/745,644

(22) Filed: May 8, 2007

Related U.S. Application Data
(60) Provisional application No. 60/886,994, filed on Jan. 29, 2007.
(60) Provisional application No. 60/866,370, filed on Nov. 17, 2006.
(60) Provisional application No. 60/798,812, filed on May 9, 2006.

(51) Int. Cl. C07K 16/00; C07H 21/00; C12N 1/00; C12N 15/64

(52) U.S. Cl. 435/243; 435/320.1; 530/387.1; 536/23.53

(57) ABSTRACT

The invention provides variant heavy chain variable domains (VH) with increased folding stability. Libraries comprising a plurality of these polypeptides are also provided. In addition, compositions and methods of generating and using these polypeptides and libraries are provided.

### RELATED APPLICATIONS

This application claims priority under 35 U.S.C. § 119(e) to U.S. provisional application No. 60/798,812, filed May 9, 2006, to U.S. provisional application No. 60/866,370, filed Nov. 17, 2006, and to U.S. provisional application No. 60/886,994, filed Jan. 29, 2007, the contents of which are incorporated in their entirety herein by reference.

### FIELD OF THE INVENTION

The invention relates to variant isolated heavy chain variable domains (VH) with increased folding stability, and libraries comprising a plurality of such molecules. The invention also relates to methods and compositions useful for identifying novel binding polypeptides that can be used therapeutically or as reagents.

### BACKGROUND

Phage display technology has provided a powerful tool for generating and selecting novel proteins that bind to a ligand, such as an antigen. Using the techniques of phage display allows the generation of large libraries of protein variants that can be rapidly sorted for those sequences that bind to a target antigen with high affinity. Nucleic acids encoding variant polypeptides are fused to a nucleic acid sequence encoding a viral coat protein, such as the gene III protein or the gene VIII protein. Monovalent phage display systems where the nucleic acid sequence encoding the protein or polypeptide is fused to a nucleic acid sequence encoding a portion of the gene III protein have been developed. (Bass, S., Proteins, 8:309 (1990); Lowman and Wells, Methods: A Companion to Methods in Enzymology, 3:205 (1991)). In a monovalent phage display system, the gene fusion is expressed at low levels and wild type gene III proteins are also expressed so that infectivity of the particles is retained. Methods of generating peptide libraries and screening those libraries have been disclosed in many patents (e.g. U.S. Pat. No. 5,723,286, U.S. Pat. No. 5,432,018, U.S. Pat. No. 5,580,717, U.S. Pat. No. 5,427,908 and U.S. Pat. No. 5,498,530).

The demonstration of expression of peptides on the surface of filamentous phage and the expression of functional antibody fragments in the periplasm of E. coli was important in the development of antibody phage display libraries. (Smith et al., Science (1985), 228:1315; Skerra and Pluckthun, Science (1988), 240:1038). Libraries of antibodies or antigen binding polypeptides have been prepared in a number of ways including by altering a single gene by inserting random DNA sequences or by cloning a family of related genes. Methods for displaying antibodies or antigen binding fragments using phage display have been described in U.S. Pat. Nos. 5,750,373, 5,733,743, 5,837,242, 5,969,108, 6,172,197, 5,580,717, and 5,658,727. The library is then screened for expression of antibodies or antigen binding proteins with desired characteristics.

Phage display technology has several advantages over conventional hybridoma and recombinant methods for preparing antibodies with the desired characteristics. This technology allows the development of large libraries of antibodies with diverse sequences in less time and without the use of animals. Preparation of hybridomas or preparation of humanized antibodies can easily require several months of preparation. In addition, since no immunization is required, phage antibody libraries can be generated for antigens which are toxic or have low antigenicity (Hoogenboom, Immunotechniques (1988), 4:1-20). Phage antibody libraries can also be used to generate and identify novel human antibodies.

Phage display libraries have been used to generate human antibodies from immunized and non-immunized humans, germ line sequences, or naïve B cell Ig repertories (Barbas & Burton, Trends Biotech (1996), 14:230; Griffiths et al., EMBO J. (1994), 13:3245; Vaughan et al., Nat. Biotech. (1996), 14:309; Winter E P 0368 684 B1). Naïve, or nonimmune, antigen binding libraries have been generated using a variety of lymphoidal tissues. Some of these libraries are commercially available, such as those developed by Cambridge Antibody Technology and Morphosys (Vaughan et al., Nature Biotech 14:309 (1996); Knappik et al., J. Mol. Biol. 296:57 (1999)). However, many of these libraries have limited diversity.

The ability to identify and isolate high affinity antibodies from a phage display library is important in isolating novel human antibodies for therapeutic use. Isolation of high affinity antibodies from a library is traditionally thought to be dependent, at least in part, on the size of the library, the efficiency of production in bacterial cells and the diversity of the library (see, e.g., Knappik et al., J. Mol. Biol. (1999), 296:57). The size of the library is decreased by inefficiency of production due to improper folding of the antibody or antigen binding protein and the presence of stop codons. Expression in bacterial cells can be inhibited if the antibody or antigen binding domain is not properly folded. Expression can be improved by mutating residues in turns at the surface of the variable/constant interface, or at selected CDR residues. (Deng et al., J. Biol. Chem. (1994), 269:9533, Ulrich et al., PNAS (1995), 92:11907-11911; Forsberg et al., J. Biol. Chem. (1997), 272:12430). The sequence of the framework region is also a factor in providing for proper folding when antibody phage libraries are produced in bacterial cells.

Antibodies have become very useful as therapeutic agents for a wide variety of conditions. For example, humanized antibodies to HER-2, a tumor antigen, are useful in the diagnosis and treatment of cancer. Other antibodies, such as anti-INF-γ antibody, are useful in treating inflammatory conditions such as Crohn's disease. Antibodies, however, are large, multichain proteins, which may pose difficulties in targeting molecules in obstructed locations and in production of the antibodies in host cells. Different antibody fragments (i.e., Fab′, F(ab)2, scFV) have been explored; most suffer the same drawbacks as full-length antibodies, but to different degrees. Recently, isolated antibody variable domains (i.e., VL, VH) have been studied.

Isolated VH or VL domains are the smallest functional antigen-binding fragments of an antibody. They are small, and thus can be used to target antigens in obstructed locations like tumors. Drug- or radioisotope-conjugated VH or VL can be more safely used in treatment because isolated VH or VL should be rapidly cleared from the system, thus minimizing contact time with the drug or radioisotope. Furthermore, isolated VH or VL can theoretically be highly expressed in bacterial cells, thus permitting increased yields and less need for costly and time-consuming mammalian cell expression. Development of VH or VL-based therapeutics have been hampered thus far by a tendency to aggregate in solution, believed to be due to the exposure to the solvent of a large hydrophobic patch that would normally associate with the other antibody chain (VH typically associates with VL in the context of a full-length antibody molecule).

Studies of single-chain antibodies lacking light chain that were discovered to naturally circulate in camel serum showed that a heavy chain is capable of recognizing and specifically binding antigen despite possessing only three of the six antigen recognition sites typically found in an antigen binding fragment having both light and heavy chains (Hamers-Casterman et al., Nature (1993) 363:446-8). The VHH domains (heavy chain variable domain of the HC antibody) of those camelid antibodies are highly soluble and expressed in large quantities in bacterial hosts. When first cloned, VHH solubility was attributed to four highly conserved mutations at the former interface with VL: Val37Tyr or Phe, Gly44Glu or Gln, Leu45Arg or Cys, and Trp47Gly or Ser, Leu, or Phe (Muyldermans et al., Protein Eng. (1994) 7:1129-35). When such mutations were introduced in human VH domains in a process known as camelisation, the modified domains aggregated less, but expression of the domains was significantly impaired (Davies et al., Biotechnology (1995) 13: 475-479). The discovery of llama VHH sequences not including the camelid conserved mutations has since further weakened support for the role of those mutations in domain solubilization and expression (Harmsen et al., Mol. Immunol. (2000) 37: 579-90; Tanha et al., J. Immunol. Methods (2002) 263:97-109; Vranken et al., Biochemistry (2002) 41:8570-79). Studies of camelid VHH also showed that their CDR-H3 was on average longer than that of human counterparts, possibly folding back onto and protecting residues from the hydrophobic interface with VL from solvent exposure (Desmyter et al., Nat. Struct. Biol. (1996) 3:803-811; Desmyter et al., J. Biol. Chem. (2002) 277:23645-50). Lengthening of CDR-H3 in camelised and human VH domains improved solubility and expression of those domains (Tanha et al., J. Biol. Chem. (2001) 276:24774-80; Ewert et al., J. Mol. Biol. (2003) 325:531-553).

Other approaches have also been attempted to improve human VH properties. Modification of the glycine at position 44 to lysine in a murine VH was reported to prevent non-specific binding and aggregation of those proteins without further camelisation at the former VL interface (Reiter et al., J. Mol. Biol. (1999) 290:685-98). Separately, improved solubility and decreased aggregation were observed in a human VH in which the histidine at position 35 was modified to glycine. (Jespers et al., J. Mol. Biol. (2004) 337: 893-903). The crystal structure of that domain showed that the side-chain of framework residue Trp47 fits into a cavity created by the removal of the side chain at position 35, in sharp contrast to the glycine at position 47 in the camel VHH. Id. Furthermore, no length modifications were made to CDR-H3 in that molecule, and it is unclear what effect lengthening CDR-H3 might have had in the context of the His35Gly mutation. Heat-selection studies have been performed to identify residues that may be involved in temperature stability (see WO2004/101790). No systematic analysis of VH modifications has yet been undertaken to understand the principles driving the conformational stability of the human VH domain, and in particular which residues support its proper folding.

VH domains appear to be ideal scaffolds for the development of synthetic phage-displayed libraries. Because of their small size and single domain nature, properly folded VH domains are likely to be highly expressed and secreted in bacterial hosts, and therefore, to be better displayed on phage than Fab or scFv. Moreover, VH domains have only three CDRs and are thus more straightforward to engineer for high specificity and affinity against a target of choice. However, as described above, the general principles and specific residues involved in proper folding of a human VH domain have not yet been ascertained. There remains a need to improve the human VH domain such that it is optimized for use in phage display libraries, where it must permit modification within the CDRs while still allowing proper folding, high levels of expression, and low aggregation. The invention described herein meets this need and provides other benefits.

### SUMMARY OF THE INVENTION

The present invention provides isolated antibody variable domains with enhanced folding stability which can serve as scaffolds for antibody construction and selection, and also provides methods of producing such antibodies. The invention is based on the surprising result that isolated heavy chain antibody variable domains can be greatly enhanced in stability by framework region modifications that decrease the hydrophobicity of the region of the heavy chain antibody variable domain that would typically interact with an antibody light chain variable domain. Certain such isolated heavy chain antibody variable domains also allow nonbiased diversification at one or more of the heavy chain complementarity determining regions (CDRs). The polypeptides and methods of the invention are useful in the isolation of high affinity binding molecules to target antigens, and the resulting well-folded antibody variable domains can readily be adapted to large scale production.

An isolated antibody variable domain is provided by the invention, wherein the antibody variable domain comprises one or more amino acid alterations as compared to the naturally-occurring antibody variable domains, and wherein the one or more amino acid alterations increase the stability of the isolated antibody variable domain. In one embodiment, the antibody variable domain is a heavy chain antibody variable domain. In one aspect, the antibody variable domain is of the VH3 subgroup. In another aspect, the increased stability of the antibody variable domain is measured by a decrease in aggregation of the antibody variable domain. In another aspect, the increased stability of the antibody variable domain is measured by an increase in Tₘ of the antibody variable domain. In another aspect, the increased stability of the antibody variable domain is measured by an increased yield in a chromatography assay. In another embodiment, the one or more amino acid alterations increase the hydrophilicity of a portion of the antibody variable domain responsible for interacting with a light chain variable domain. In one aspect, the VH domain prior to mutation has the sequence of SEQ ID NO: 1. In another aspect, the VH domain prior to mutation has the sequence of SEQ ID NO: 2.

In one embodiment, an isolated heavy chain antibody variable domain is provided wherein the heavy chain antibody variable domain comprises one or more amino acid alterations as compared to the naturally-occurring heavy chain antibody variable domain, and wherein the one or more amino acid alterations increase the stability of the isolated heavy chain antibody variable domain, and wherein the one or more amino acid alterations are selected from alterations at amino acid positions 35, 37, 45, 47, and 93-102. In one aspect, amino acid position 35 is alanine, amino acid position 45 is valine, amino acid position 47 is methionine, amino acid position 93 is threonine, amino acid position 94 is serine, amino acid position 95 is lysine, amino acid position 96 is lysine, amino acid position 97 is lysine, amino acid position 98 is serine, amino acid position 99 is serine, amino acid position 100 is proline, and amino acid position 100a is isoleucine. In another aspect, the isolated heavy chain antibody variable domain has an amino acid sequence comprising SEQ ID NOs: 28 and 54. In another aspect, amino acid position 35 is glycine, amino acid position 45 is tyrosine, amino acid position 93 is arginine, amino acid position 94 is threonine, amino acid position 95 is phenylalanine, amino acid position 96 is threonine, amino acid position 97 is threonine, amino acid position 98 is asparagine, amino acid position 99 is serine, amino acid position 100 is lysine, and amino acid position 100a is lysine. In another aspect, the isolated heavy chain antibody variable domain has an amino acid sequence comprising SEQ ID NOs: 26 and 52. In another aspect, amino acid position 35 is serine, amino acid position 37 is alanine, amino acid position 45 is methionine, amino acid position 47 is serine, amino acid position 93 is valine, amino acid position 94 is threonine, amino acid position 95 is glycine, amino acid position 96 is asparagine, amino acid position 97 is arginine, amino acid position 98 is threonine, amino acid position 99 is leucine, amino acid position 100 is lysine, and amino acid position 100a is lysine. In another aspect, the isolated heavy chain antibody variable domain has an amino acid sequence comprising SEQ ID NOs: 31 and 57. In another aspect, amino acid position 35 is serine, amino acid position 45 is arginine, amino acid position 47 is glutamic acid, amino acid position 93 is isoleucine, amino acid position 95 is lysine, amino acid position 96 is leucine, amino acid position 97 is threonine, amino acid position 98 is asparagine, amino acid position 99 is arginine, amino acid position 100 is serine, and amino acid position 100a is arginine. In another aspect, the isolated heavy chain antibody variable domain has an amino acid sequence comprising SEQ ID NOs: 39 and 65. In one aspect, the VH domain prior to mutation has the sequence of SEQ ID NO: 1. In another aspect, the VH domain prior to mutation has the sequence of SEQ ID NO: 2.

In another aspect, the amino acid at amino acid position 35 is a small amino acid. In another aspect, the small amino acid is selected from glycine, alanine, and serine. In another aspect, the amino acid at amino acid position 37 is a hydrophobic amino acid. In another aspect, the hydrophobic amino acid is selected from tryptophan, phenylalanine, and tyrosine. In another aspect, the amino acid at amino acid position 45 is a hydrophobic amino acid. In another aspect, the hydrophobic amino acid is selected from tryptophan, phenylalanine, and tyrosine. In another aspect, amino acid position 35 is selected from glycine and alanine and amino acid position 47 is selected from tryptophan and methionine. In another aspect, amino acid position 35 is serine, and amino acid position 47 is selected from phenylalanine and glutamic acid. In one aspect, the VH domain prior to mutation has the sequence of SEQ ID NO: 1. In another aspect, the VH domain prior to mutation has the sequence of SEQ ID NO: 2.

In another embodiment, an isolated heavy chain antibody variable domain is provided wherein the heavy chain antibody variable domain comprises one or more amino acid alterations selected from alterations at amino acid positions 35, 37, 39, 44, 45, 47, 50, 91, 93-10b, 103, and 105 as compared to the naturally-occurring heavy chain antibody variable domain, wherein the one or more amino acid alterations increase the stability of the isolated heavy chain antibody variable domain. In one aspect, amino acid position 35 is glycine, amino acid position 39 is arginine, amino acid position 45 is glutamic acid, amino acid position 50 is serine, amino acid position 93 is arginine, amino acid position 94 is serine, amino acid position 95 is leucine, amino acid position 96 is threonine, amino acid position 97 is threonine, amino acid position 99 is serine, amino acid position 100 is lysine, amino acid position 100a is threonine, and amino acid position 103 is arginine. In another aspect, the isolated heavy chain antibody variable domain has an amino acid sequence comprising SEQ ID NOs: 139 and 215. In another aspect, the amino acid at any of amino acid positions 39, 45, and 50 is a hydrophilic amino acid. In another aspect, each of the amino acids at amino acid positions 39, 45, and 50 are hydrophilic amino acids. In another aspect, amino acid position 39 is arginine, amino acid position 45 is glutamic acid, and amino acid position 50 is serine. In another aspect, each of the amino acids at amino acid positions 39, 45, and 50 are hydrophilic amino acids. In another aspect, amino acid position 39 is arginine, amino acid position 45 is glutamic acid, and amino acid position 50 is serine. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided wherein the heavy chain antibody variable domain comprises one or more amino acid alterations as compared to the naturally-occurring antibody variable domain, wherein amino acid positions 37, 44, and 91 are wild-type, and wherein the one or more amino acid alterations increase the stability of the isolated heavy chain antibody variable domain. In one aspect, the isolated heavy chain antibody variable domain is tolerant to substitution at each amino acid position in CDR-H3. In another aspect, the isolated heavy chain antibody variable domain has an amino acid sequence comprising SEQ ID NO: 26. In another aspect, the isolated heavy chain antibody variable domain has an amino acid sequence comprising SEQ ID NO: 139. In another aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the heavy chain antibody variable domain comprises one or more amino acid alterations at amino acid positions 35, 37, 39, 44, 45, 47, 50, and 91 as compared to the naturally-occurring heavy chain antibody variable domain, and wherein the one or more amino acid alterations increase the stability of the isolated heavy chain antibody variable domain. In one aspect, the amino acid at amino acid position 35 is selected from glycine, alanine, serine, and glutamic acid; the amino acid at amino acid position 39 is glutamic acid; and the amino acid at amino acid position 50 is selected from glycine and arginine, and wherein the amino acids at amino acid positions 37, 44, 47, and 91 are wild-type. In another aspect, the amino acid at amino acid position 35 is glycine, the amino acid at amino acid position 37 is a hydrophobic amino acid; the amino acid at amino acid position 39 is arginine; the amino acid at amino acid position 44 is a small amino acid; the amino acid at amino acid position 45 is glutamic acid; the amino acid at amino acid position 47 is selected from leucine, valine, and alanine; the amino acid at amino acid position 50 is serine; and the amino acid at amino acid position 91 is a hydrophobic amino acid. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the amino acid at amino acid position 35 is glycine; wherein the amino acid at amino acid position 39 is arginine; wherein the amino acid at amino acid position 45 is glutamic acid; wherein the amino acid at amino acid position 47 is leucine; and wherein the amino acid at amino acid position 50 is arginine. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the isolated heavy chain antibody variable domain comprises one or more amino acid alterations as compared to the naturally-occurring heavy chain antibody variable domain, wherein the one or more amino acid alterations increase the stability of the isolated heavy chain antibody variable domain, and wherein the heavy chain antibody variable domain has an amino acid sequence comprising SEQ ID NO: 26. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the heavy chain antibody variable domain comprises one or more amino acid alterations as compared to the naturally-occurring heavy chain antibody variable domain, wherein the one or more amino acid alterations increase the stability of the isolated heavy chain antibody variable domain, and wherein the heavy chain antibody variable domain has an amino acid sequence comprising SEQ ID NO: 139. In one aspect, the heavy chain antibody variable domain further comprises an alteration at amino acid position 35. In another such aspect, the amino acid at amino acid position 35 is selected from glycine, serine and aspartic acid. In another aspect, the heavy chain antibody variable domain further comprises an alteration at amino acid position 39. In another such aspect, the amino acid at amino acid position 39 is aspartic acid. In another aspect, the heavy chain antibody variable domain further comprises an alteration at amino acid position 47. In another such aspect, the amino acid at amino acid position 47 is selected from alanine, glutamic acid, leucine, threonine, and valine. In another aspect, the heavy chain antibody variable domain further comprises an alteration at amino acid position 47 and another amino acid position. In another such aspect, the amino acid at amino acid position 47 is glutamic acid and the amino acid at amino acid position 35 is serine. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the framework regions of the antibody variable domain comprise two amino acid alterations as compared to the naturally-occurring antibody variable domain, and wherein the two amino acid alterations increase the stability of the antibody variable domain. In one embodiment, the heavy chain antibody variable domain comprises a leucine at amino acid position 47 and a threonine at amino acid position 37. In another embodiment, the heavy chain antibody variable domain comprises a leucine at amino acid position 47 and an amino acid at amino acid position 39 selected from serine, threonine, lysine, histidine, glutamine, aspartic acid, and glutamic acid. In another embodiment, the heavy chain antibody variable domain comprises a leucine at amino acid position 47 and an amino acid at amino acid position 45 selected from serine, threonine, and histidine. In another embodiment, the heavy chain antibody variable domain comprises a leucine at amino acid position 47 and an amino acid at amino acid position 103 selected from serine and threonine. In another embodiment, the heavy chain antibody variable domain comprises a glycine at amino acid position 35, an arginine at amino acid position 39, a glutamic acid at amino acid position 45, a leucine at amino acid position 47, and a serine at amino acid position 50. In one aspect, the heavy chain antibody variable domain further comprises a serine at amino acid position 37. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the framework regions of the antibody variable domain comprise three amino acid alterations as compared to the naturally-occurring antibody variable domain, and wherein the three amino acid alterations increase the stability of the antibody variable domain. In one embodiment, the heavy chain antibody variable domain comprises three mutations selected from V37S, W47L, S50R, W103S, and W103R. In another embodiment, the heavy chain antibody variable domain comprises a leucine at amino acid position 47 and two mutations selected from V37S, S50R, and W103S. In another embodiment, the heavy chain antibody variable domain comprises a leucine at amino acid position 47 and two mutations selected from V37S, S50R, and W103R. In one aspect, the VH domain prior to mutation has the sequence of SEQ ID NO: 1. In another aspect, the VH domain prior to mutation has the sequence of SEQ ID NO: 2.

An isolated heavy chain antibody variable domain is provided, wherein the framework regions of the antibody variable domain comprise four amino acid alterations as compared to the naturally-occurring antibody variable domain, and wherein the four amino acid alterations increase the stability of the antibody variable domain. In one embodiment, the heavy chain antibody variable domain comprises a serine at amino acid position 37, a leucine at amino acid position 47, an arginine at amino acid position 50, and an amino acid at amino acid position 103 selected from serine and arginine. In another embodiment, the heavy chain antibody variable domain comprises a serine at amino acid position 37, a leucine at amino acid position 47, an arginine at amino acid position 50, and an arginine at amino acid position 103. In another embodiment, the heavy chain antibody variable domain comprises a serine at amino acid position 37, a leucine at amino acid position 47, an arginine at amino acid position 50, and a serine at amino acid position 103. In one aspect, the VH domain prior to mutation has the sequence of SEQ ID NO: 1. In another aspect, the VH domain prior to mutation has the sequence of SEQ ID NO: 2.

In another embodiment, the invention provides an isolated heavy chain antibody variable domain comprising mutations at amino acid positions 35, 39, and 45, and further comprising one or more amino acid mutations at amino acid positions selected from 37, 47, 50, and 103. In one aspect, the mutations at amino acid positions 35, 39, and 45 are H35G, Q39R, and L45E. In another aspect, the one or more amino acid mutations at amino acid positions selected from 37, 47, 50, and 103 are selected from V37S, W47L, S50R, W103R, and W103S. In another aspect, the VH domain prior to mutation has the sequence of SEQ ID NO: 1. In another aspect, the VH domain prior to mutation has the sequence of SEQ ID NO: 2.

In another embodiment, the invention provides an isolated heavy chain antibody variable domain comprising mutations at amino acid positions 35, 39, and 45, and 50, and further comprising one or more amino acid mutations at amino acid positions selected from 37, 47, and 103. In one aspect, the mutations at amino acid positions 35, 39, 45, and 50 are H35G, Q39R, L45E, and R50S. In another aspect, the one or more amino acid mutations at amino acid positions selected from 37, 47, and 103 are selected from V37S, W47L, W103R, and W103S. In another aspect, the VH domain prior to mutation has the sequence of SEQ ID NO: 1. In another aspect, the VH domain prior to mutation has the sequence of SEQ ID NO: 2.

In another embodiment, a polynucleotide encoding any of the foregoing antibody variable domains is provided. In another embodiment, a replicable expression vector comprising such a polynucleotide is provided. In another embodiment, a host cell comprising such a replicable expression vector is provided. In another embodiment, a library of such replicable expression vectors is provided. In another embodiment, a plurality of any of the foregoing antibody variable domains is provided. In one aspect, each antibody variable domain of the plurality of antibody variable domains comprises one or more variant amino acids in at least one complementarity determining region (CDR). In one such aspect, the at least one complementarity determining region is selected from CDR-H1, CDR-H2, and CDR-H3.

In another embodiment, a composition comprising any of the foregoing antibody variable domains is provided. In one aspect, the composition further comprises a suitable diluent. In another aspect, the composition further comprises one or more additional therapeutic agents. In another such aspect, the one or more additional therapeutic agents comprise at least one chemotherapeutic agent. In another embodiment, a kit is provided, comprising any of the foregoing antibody variable domains. In one aspect, the kit further comprises one or more additional therapeutic agents. In another aspect, the kit further comprises instructions for use.

In another embodiment, a method of generating a plurality of isolated heavy chain antibody variable domains is provided, comprising altering one or more framework regions of the heavy chain antibody variable domain as compared to the naturally-occurring heavy chain antibody variable domain, wherein the one or more amino acid alterations increases the stability of the heavy chain antibody variable domain. In one aspect, the one or more amino acid alterations are amino acid alterations described herein.

In another embodiment, any of the above-described isolated heavy chain antibody variable domains may be modular binding units in bispecific or multi-specific antibodies.

In another embodiment, a method of increasing the stability of an isolated heavy chain antibody variable domain is provided, comprising altering one or more framework amino acids of the antibody variable domain as compared to the naturally-occurring antibody variable domain, wherein the one or more framework amino acid alterations increases the stability of the isolated heavy chain antibody variable domain. In one aspect, the one or more amino acid alterations are amino acid alterations described herein.

### BRIEF DESCRIPTION OF THE FIGURES

FIG. 1A depicts the nucleotide (SEQ ID NOs. 269 and 270) and amino acid (SED ID NO: 1) sequences of the 4D5 heavy chain variable domain (VH), with the Protein A-binding sequences and CDR-H1, CDR-H2, and CDR-H3 indicated. FIG. 1B depicts the nucleotide (SEQ ID NOs. 271 and 272) and amino acid sequences (SEQ ID NO: 2) of the 4D5 heavy chain variable domain used to construct the Lib2_{—}3 mutants described in Example 4, which differs from the sequence in FIG. 1A at four amino acids underlined).

FIG. 2 schematically illustrates the arrangement of genetic elements and the human 4D5 VH domain coding sequence in plasmid pPAB43431-7.

FIG. 3 depicts the crystallographic structure of the wild-type VL and VH domains from the 4D5 monoclonal antibody (left image). The enlarged VH domain (right image) shows the different regions of the 4D5 VH domain that interact with Protein A or VL.

FIGS. 4A and 4B show the wild-type 4D5 VH domain amino acid sequence and each of the 25 unique amino acid sequences obtained from Library 1 selectants, as described in Example 1. Each of the Library 1 sequences was identical to the wild-type sequence at all positions not otherwise indicated. The boxed residues indicate groupings of sequences based on the residue at position 35 (glycine, alanine, or serine).

FIG. 5 shows a bar graph of the purification yields for each of Library 1 VH domain selectants Lib1_{—}17, Lib1_{—}62, Lib1_{—}87, Lib1_{—}90, Lib1_{—}45, and Lib1_{—}66 in comparison with the wild-type 4D5 VH domain, as described in Example 1D(1).

FIGS. 6A-6D show traces from gel filtration/light scattering analyses of the wild-type 4D5 VH domain and each of Library 1 VH domain selectants Lib117, Lib1_{—}62, Lib1_{—}87, Lib1_{—}90, Lib1_{—}45, and Lib1_{—}66, as described in Example 1D(2).

FIG. 7 shows melting curves over a 25-85° C. range for the wild-type 4D5 VH domain (“WT”) and for each of Library 1 VH domain selectants Lib1_{—}17, Lib1_{—}62, Lib1_{—}87, Lib1_{—}90, Lib1_{—}45, and Lib1_{—}66, as described in Example 1D(3). The light line indicates the refolding transition, where the temperature was decreased from 85° C. to 25° C. The heavy line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

FIG. 8 shows a graph depicting the results of the Protein A ELISA assay described in Example 1E.

FIGS. 9A-9D show the wild-type 4D5 VH domain amino acid sequence and each of the 74 unique amino acid sequences obtained from Library 2 selectants, as described in Example 2. Each of the Library 2 sequences was identical to the wild-type sequence at all positions not otherwise indicated.

FIGS. 10A and 10B depict the results from experiments assessing the ability of Library 2 selectants to bind to Protein A, as described in Example 2. FIG. 10A shows a bar graph of the purification yields obtained using column chromatography with Protein A-conjugated resin for the wild-type 4D5 VH domain, Lib1_{—}62, and eleven Library 2 clones of interest. FIG. 10B shows the results of a Protein A ELISA for wild-type 4D5 VH domain, Lib1_{—}62, and eleven Library 2 clones of interest.

FIG. 11 shows traces from gel filtration/light scattering analyses of the wild-type 4D5 VH domain and the Lib2_{—}3 VH domain, as described in Example 2.

FIG. 12 shows melting curves over a 25-85° C. range for the wild-type 4D5 VH domain (“WT”) and for the Lib2_{—}3 VH domain, as described in Example 2. The light line indicates the refolding transition, where the temperature was decreased from 85° C. to 25° C. The heavy line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

FIG. 13 shows two tables corresponding to the randomized residues from Library 2 that were wild-type (V37, G44, W47, and Y91) or mutagenic (H35G, Q39R, L45E, and R50S) in the Lib2_{—}3 VH domain. The tables list the number of times that a particular one of the twenty amino acids appeared in the sequences obtained from Libraries 3 and 4, as described in Example 3. Light shading denotes that the amino acid was prevalent at the indicated position, while a darker shading denotes that the amino acid had a low incidence at the indicated position. “TH” indicates transformed Shannon entropy.

FIG. 14 shows a bar graph depicting the wild-type/alanine ratio at each of the VH domain CDR-H3 positions alanine scanned in Library 5, as described in Example 5.

FIGS. 15A-C show traces from gel filtration/light scattering analyses of the amber Lib2_{—}3 mutant and each of Lib2_{—}3.4D5H3.G35S, Lib2_{—}3.4D5H3.R39D, Lib2_{—}3.4D5H3.W47A, Lib2_{—}3.4D5H3.W47E, Lib2_{—}3.4D5H3.W47L, Lib2_{—}3.4D5H3.W47T, Lib2_{—}3.4D5H3.W47V, and Lib2_{—}3.4D5H3.W47E, as described in Example 4.

FIGS. 16A and 16B show melting curves over a 25-85° C. range for WT 4D5, the Lib2_{—}3 amber mutant, Lib2_{—}3.4D5H3.W47A, Lib2_{—}3.4D5H3.W47E, Lib2_{—}3.4D5H3.W47L, Lib2_{—}3.4D5H3.W47T, Lib2_{—}3.4D5H3.W47V, Lib2_{—}3.4D5H3.W47E, as described in Example 4. The dotted line indicates the refolding transition, where the temperature was decreased from 85° C. to 25° C. The solid line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

FIGS. 17A-D show traces from gel filtration/light scattering analyses of each of Lib2_{—}3.4D5H3.W47L/V37S, Lib2_{—}3.4D5H3.W47L/V37T, Lib2_{—}3.4D5H3.W47L/R39S, Lib2_{—}3.4D5H3.W47L/R39T, Lib2_{—}3.4D5H3.W47L/R39K, Lib2_{—}3.4D5H3.W47L/R39H, Lib2_{—}3.4D5H3.W47L/R39Q, and Lib2_{—}3.4D5H3.W47L/R39D, Lib2_{—}3.4D5H3.W47L/R39E Lib2_{—}3.4D5H3.W47L/E45 S Lib2_{—}3.4D5H3.W47L/E45T Lib2_{—}3.4D5H3.W47L/E45H, Lib2_{—}3.4D5H3.W47L/W103 S, Lib2_{—}3.4D5H3.W47L/W103T, and Lib2_{—}3.4D5H3.W47L/W47L, as described in Example 4.

FIG. 18 shows melting curves over a 25-85° C. range for Lib2_{—}3.4D5H3.W47L/V37S, as described in Example 4. The dotted line indicates the refolding transition, where the temperature was decreased from 85° C. to 25° C. The solid line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

FIG. 19 shows the results of a Protein A ELISA for wild-type 4D5 VH domain, the 4D5 Fab, Lib1_{—}62, Lib1_{—}90, Lib2_{—}3, Lib2_{—}3 with a wild-type 4D5H3 domain, and Lib2_{—}3.4D5H3.T57E.

FIGS. 20A and 20B show crystal structures of various VH and VHH domains, as described in Example 6. FIG. 20A shows the structure of the Herceptin® VH domain (left panel), as described in Cho et al. (Nature. (2003) February 13; 421(6924):756-60), and the structure of VH-B1a. The VH-B1a structure has a resolution of 1.7 Å, R_{(cryst)} of 16.4%, R_{(free)} of 20.4%, and a root mean square deviation (calculated with framework Calpha atoms of the 1N8Z VH domain for molecular replacement) of 0.65° (based on 108/120 residues). FIG. 20B shows detail views of the region surrounding residue 35 of the crystal structures obtained for a camelid anti-human chorionic gonadotropin VHH domain (Bond et al., J. Mol. Biol. 332: 643-655 (2003)) (upper left panel), a HEL-binding VH domain (VH-Hel4) (Jespers et al., J. Mol. Biol. 337: 893-903 (2004)) (upper right panel), the Herceptin VH domain (bottom left panel) and VH-B1a (bottom right panel).

FIG. 21 shows traces from gel filtration/light scattering analyses of two different concentrations of VH domain B1a, as described in Example 7a.

FIGS. 22A and 22B show traces from gel filtration/light scattering analyses of different oligomeric states of B1a, as described in Example 7a.

FIG. 23 shows the results from reducing and non-reducing SDS-polyacrylamide gel electrophoresis analyses of different oligomeric states of B1a, as described in Example 7a.

FIGS. 24A-B show a table providing protein yield, extinction coefficient, molecular weight, peak area, retention time, melting temperature and refolding percentage data for many VH domains described herein (see, e.g., Example 7B and Example 8).

FIGS. 25A-25F show traces from gel filtration/light scattering analyses of mutant B1a VH domains, as described in Example 7b.

FIGS. 26A-26H shows graphs of the percentage of folding observed upon increase (solid line) and decrease (broken line) of temperature for certain VH domains described herein, as described in Example 7b.

FIGS. 27A-27D show melting curves over a 25-85° C. range for the B1a VH domain and several B1a mutant VH domains, as described in Example 7b. The dotted line indicates the refolding transition, where the temperature was decreased from 85° C. to 25° C. The solid line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

FIGS. 28A-28C show traces from gel filtration/light scattering analyses of mutant VH domains, as described in Example 8.

FIGS. 29A-29C show graphs of the percentage of folding observed upon increase (top, solid line) and decrease (bottom, broken line) of temperature for certain VH domains described herein, as described in Example 8.

FIGS. 30A-30C show melting curves over a 25-85° C. range for certain B1a mutant VH domains, as described in Example 8. The dotted line indicates the refolding transition, where the temperature was decreased from 85° C. to 25° C. The solid line depicts the unfolding transition, where the temperature was increased from 25° C. to 95° C. The reversibility of the phenomenon was assessed by placing the protein sample at 85° C., followed by cooling down the protein sample from 85° C. to 25° C. and then heating it again to 95° C.

### DISCLOSURE OF THE INVENTION

A. Definitions

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “antibody” is used in the broadest sense and specifically covers single monoclonal antibodies (including agonist and antagonist antibodies), antibody compositions with polyepitopic specificity, affinity matured antibodies, humanized antibodies, chimeric antibodies, single chain antigen binding molecules such as monobodies, as well as antigen binding fragments or polypeptides (e.g., Fab, F(ab′)₂, scFv and Fv), so long as they exhibit the desired biological activity.

As used herein, “antibody variable domain” refers to the portions of the light and heavy chains of antibody molecules that include amino acid sequences of Complementary Determining Regions (CDRs; ie., CDR1, CDR2, and CDR3), and Framework Regions (FRs; i.e. FR1, FR2, FR3, and FR4). A FR includes those amino acid positions in an antibody variable domain other than CDR positions as defined herein. VH refers to the variable domain of the heavy chain of an antibody. VL refers to the variable domain of the light chain of an antibody. VHH refers to the heavy chain variable domain of a monobody. According to the methods used in this invention, the amino acid positions assigned to CDRs and FRs are defined according to Kabat (Sequences of Proteins of Immunological Interest (National Institutes of Health, Bethesda, Md., 1987 and 1991)). Amino acid numbering of antibodies or antigen binding fragment thereof is also according to that of Kabat et al. cited supra.

As used herein “CDR” refers to a contiguous sequence of amino acids that form a loop in an antigen binding pocket or groove. The amino acid sequences included in a CDR loop are selected based on structure or amino acid sequence. In an embodiment, the loop amino acids of a CDR are determined by inspection of the three-dimensional structure of an antibody, antibody heavy chain, or antibody light chain. The three-dimensional structure may be analyzed for solvent accessible amino acid positions as such positions are likely to form a loop in an antibody variable domain. The three dimensional structure of the antibody variable domain may be derived from a crystal structure or protein modeling. In another embodiment, the loop boundaries of the CDR are determined according to Chothia (Chothia and Lesk, 1987, J. Mol. Biol., 196:901-917). One to three amino acid residues may optionally be added to the C-terminal and N-terminal ends of the Chothia CDRs. In some embodiments, the amino acid positions of CDR1 comprise, consist essentially of or consist of amino acid positions 24 to 34, the amino acid positions of CDR2 comprise, consist essentially of or consist of amino acid positions 51 to 56 and the CDR3 positions comprise, consist essentially of or consist of amino acid positions 96 to 101 of an antibody heavy chain variable domain.

“Antibody fragments” comprise only a portion of an intact antibody, generally including an antigen binding site of the intact antibody and thus retaining the ability to bind antigen. Nonlimiting examples of antibody fragments encompassed by the present definition include: (i) the Fab fragment, having VL, CL, VH and CH1 domains having one interchain disulfide bond between the heavy and light chain; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment which consists of a VH domain; (vii) hingeless antibodies including at least VL, VH, CL, CH1 domains and lacking hinge region; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (VH) connected to a light chain variable domain (VL) in the same polypeptide chain; (xi) single arm antigen binding molecules comprising a light chain, a heavy chain and a N-terminally truncated heavy chain constant region sufficient to form a Fc region capable of increasing the half life of the single arm antigen binding domain; and (xii) “linear antibodies” comprising a pair of tandem Fd segments (VH-CH₁-VH-CH1) which, together with complementary light chain polypeptides, form a pair of antigen binding regions.

The term “monobody” as used herein, refers to an antigen binding molecule with at least one heavy chain variable domain and no light chain variable domain. A monobody can bind to an antigen in the absence of light chains and typically has three CDR regions designated CDRH1, CDRH2 and CDRH3. A heavy chain IgG monobody has two heavy chain antigen binding molecules connected by a disulfide bond. The heavy chain variable domain comprises one or more CDR regions, e.g., a CDRH3 region.

A “Vₕ” or “VH” or “VH domain” refers to a variable domain of an antibody heavy chain. A “VL” or “VL” or “VL domain” refers to a variable domain of an antibody light chain. A “VHH” or a “VₕH” refers to a variable domain of a heavy chain antibody that occurs in the form of a monobody. A “camelid monobody” or “camelid VHH” refers to a monobody or antigen binding portion thereof obtained from a source animal of the camelid family, including animals having feet with two toes and leathery soles. Animals in the camelid family include, but are not limited to, camels, llamas, and alpacas.

The term “monoclonal antibody” as used herein refers to an antibody obtained from a population of substantially homogeneous antibodies, i.e., the individual antibodies comprising the population are essentially identical except for variants that may arise during production of the antibody.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region (HVR) of the recipient are replaced by residues from a hypervariable region (HVR) of a non-human species (donor antibody) such as mouse, rat, rabbit or nonhuman primate having the desired specificity, affinity, and capacity. In some instances, framework region (FR) residues of the human immunoglobulin are replaced by corresponding non-human residues to improve antigen binding affinity. Furthermore, humanized antibodies may comprise residues that are not found in the recipient antibody or the donor antibody. These modifications may be made to improve antibody affinity or functional activity. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. Humanized antibodies can also be produced as antigen binding fragments as described herein. The humanized antibody optionally will also comprise at least a portion of an immunoglobulin constant region (Fc), typically that of or derived from a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

As used herein, “highly diverse position” refers to a position of an amino acid located in the variable regions of an antibody light or heavy chain that has a number of different amino acid represented at the position when the amino acid sequences of known and/or naturally occurring antibodies or antigen binding fragment or polypeptides are compared. The highly diverse positions are typically found in the CDR regions. In one aspect, the ability to determine highly diverse positions in known and/or naturally occurring antibodies is facilitated by the data provided by Kabat, Sequences of Proteins of Immunological Interest (National Institutes of Health, Bethesda, Md., 1987 and 1991). An Internet-based database located at http://www.bioinf.org.uk/abs/simkab.html provides an extensive collection and alignment of human light and heavy chain sequences and facilitates determination of highly diverse positions in these sequences. According to the invention, an amino acid position is highly diverse if it has preferably from about 2 to about 11, preferably from about 4 to about 9, and preferably from about 5 to about 7 different possible amino acid residue variations at that position. In some embodiments, an amino acid position is highly diverse if it has preferably at least about 2, preferably at least about 4, preferably at least about 6, and preferably at least about 8 different possible amino acid residue variations at that position.

As used herein, “library” refers to a plurality of antibody, antibody fragment sequences, or antibody variable domains (for example, polypeptides of the invention), or the nucleic acids that encode these sequences, the sequences being different in the combination of variant amino acids that are introduced into these sequences according to the methods of the invention.

A “scaffold”, as used herein, refers to a polypeptide or portion thereof that maintains a stable structure or structural element when a heterologous polypeptide is inserted into the polypeptide. The scaffold provides for maintenance of a structural and/or functional feature of the polypeptide after the heterologous polypeptide has been inserted. In one embodiment, a scaffold comprises one or more FR regions of an antibody variable domain, and maintains a stable structure when a heterologous CDR is inserted into the scaffold.

A “source antibody”, as used herein, refers to an antibody or antigen binding polypeptide whose antigen binding determinant sequence serves as the template sequence upon which diversification according to the criteria described herein is performed. A source antibody variable domain can include an antibody, antibody variable domain, antigen binding fragment or polypeptide thereof, a monobody, VHH, a monobody or antibody variable domain obtained from a naïve or synthetic library, camelid antibodies, naturally occurring antibody or monobody, synthetic antibody, or recombinant antibody, humanized antibody or monobody, germline derived antibody or monobody, chimeric antibody or monobody, and affinity matured antibody or monobody. In one embodiment, the polypeptide is an antibody variable domain that is a member of the Vh3 subgroup.

As used herein, “solvent accessible position” refers to a position of an amino acid residue in the variable region of a heavy and/or light chain of a source antibody or antigen binding polypeptide that is determined, based on structure, ensemble of structures and/or modeled structure of the antibody or antigen binding polypeptide, as potentially available for solvent access and/or contact with a molecule, such as an antibody-specific antigen. These positions are typically found in the CDRs, but can also be found in FR and on the exterior surface of the protein. The solvent accessible positions of an antibody or antigen binding polypeptide, as defined herein, can be determined using any of a number of algorithms known in the art. In certain embodiments, solvent accessible positions are determined using coordinates from a 3-dimensional model of an antibody or antigen binding polypeptide, e.g., using a computer program such as the InsightII program (Accelrys, San Diego, Calif.). Solvent accessible positions can also be determined using algorithms known in the art (e.g., Lee and Richards, J. Mol. Biol. 55, 379 (1971) and Connolly, J. Appl. Cryst. 16, 548 (1983)). Determination of solvent accessible positions can be performed using software suitable for protein modeling and 3-dimensional structural information obtained from an antibody. Software that can be utilized for these purposes includes SYBYL Biopolymer Module software (Tripos Associates). Generally, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers.” Comput. Chem. 18(4): 377-386; and (1995). “Variations of Surface Areas and Volumes in Distinct Molecular Surfaces of Biomolecules.” J. Mol. Model. 1: 46-53.)

The phrase “structural amino acid position” as used herein refers to an amino acid of a polypeptide that contributes to the stability of the structure of the polypeptide such that the polypeptide retains at least one biological function such as specifically binding to a molecule e.g., an antigen or a target molecule. Structural amino acid positions are identified as amino acid positions less tolerant to amino acid substitutions without affecting the structural stability of the polypeptide. Amino acid positions less tolerant to amino acid substitutions can be identified using a method such as alanine scanning mutagenesis or shotgun scanning as described in WO 01/44463 and analyzing the effect of loss of the wild type amino acid on structural stability.

The term “stability” as used herein refers to the ability of a molecule to maintain a folded state under physiological conditions such that it retains at least one of its normal functional activities, for example, binding to an antigen or to a molecule like Protein A. The stability of the molecule can be determined using standard methods. For example, the stability of a molecule can be determined by measuring the thermal melt (“Tₘ”) temperature. The Tₘ is the temperature in degrees Celsius at which ½ of the molecules become unfolded. Typically, the higher the Tₘ, the more stable the molecule.

The phrase “randomly generated population” as used herein refers to a population of polypeptides wherein one or more amino acid positions in a domain has a variant amino acid encoded by a random codon set which allows for substitution of all 20 naturally occurring amino acids at that position. For example, in one embodiment, a randomly generated population of polypeptides having randomized VH or portions thereof includes a variant amino acid at each position in the VH that is encoded by a random codon set. A random codon set includes but is not limited to codon sets designated NNS and NNK. “Cell”, “cell line”, and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

The term “coat protein” means a protein, at least a portion of which is present on the surface of the virus particle. From a functional perspective, a coat protein is any protein, which associates with a virus particle during the viral assembly process in a host cell, and remains associated with the assembled virus until it infects another host cell. The coat protein may be the major coat protein or may be a minor coat protein. A “major” coat protein is generally a coat protein which is present in the viral coat at preferably at least about 5, more preferably at least about 7, even more preferably at least about 10 copies of the protein or more. A major coat protein may be present in tens, hundreds or even thousands of copies per virion. An example of a major coat protein is the p8 protein of filamentous phage.

As used herein, “codon set” refers to a set of different nucleotide triplet sequences used to encode desired variant amino acids. A set of oligonucleotides can be synthesized, for example, by solid phase synthesis, containing sequences that represent all possible combinations of nucleotide triplets provided by the codon set and that will encode the desired group of amino acids. A standard form of codon designation is that of the IUB code, which is known in the art and described herein. A “non-random codon set”, as used herein, thus refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et al.; J. Mol. Biol. (1999), 296:57-86); Garrard & Henner, Gene (1993), 128:103). Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can, but does not necessarily, include restriction enzyme sites useful for, for example, cloning purposes.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target molecule, catalysis of a reaction, etc. The two portions may be linked directly by a single peptide bond or through a peptide linker containing one or more amino acid residues. Generally, the two portions and the linker will be in reading frame with each other.

“Heterologous DNA” is any DNA that is introduced into a host cell. The DNA may be derived from a variety of sources including genomic DNA, cDNA, synthetic DNA and fusions or combinations of these. The DNA may include DNA from the same cell or cell type as the host or recipient cell or DNA from a different cell type, for example, from a mammal or plant. The DNA may, optionally, include marker or selection genes, for example, antibiotic resistance genes, temperature resistance genes, etc.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

A “mutation” is a deletion, insertion, or substitution of a nucleotide(s) relative to a reference nucleotide sequence, such as a wild type sequence.

As used herein, “natural” or “naturally occurring” polypeptides or polynucleotides refers to a polypeptide or a polynucleotide having a sequence of a polypeptide or a polynucleotide identified from a nonsynthetic source. For example, when the polypeptide is an antibody or antibody fragment, the nonsynthetic source can be a differentiated antigen-specific B cell obtained ex vivo, or its corresponding hybridoma cell line, or from the serum of an animal. Such antibodies can include antibodies generated in any type of immune response, either natural or otherwise induced. Natural antibodies include the amino acid sequences, and the nucleotide sequences that constitute or encode these antibodies, for example, as identified in the Kabat database. As used herein, natural antibodies are different than “synthetic antibodies”, synthetic antibodies referring to antibody sequences that have been changed, for example, by the replacement, deletion, or addition, of an amino acid, or more than one amino acid, at a certain position with a different amino acid, the different amino acid providing an antibody sequence different from the source antibody sequence.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

“Phage display” is a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently sorted for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991).

A “phagemid” is a plasmid vector having a bacterial origin of replication, e.g., Co1E1, and a copy of an intergenic region of a bacteriophage. The phagemid may be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids, which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. The phage can be a filamentous bacteriophage, such as an M13, f1, fd, Pf3 phage or a derivative thereof, or a lambdoid phage, such as lambda, 21, phi80, phi81, 82, 424, 434, etc., or a derivative thereof.

“Oligonucleotides” are short-length, single- or double-stranded polydeoxynucleotides that are prepared by known methods such as chemical synthesis (e.g. phosphotriester, phosphite, or phosphoramidite chemistry, using solid-phase techniques such as described in EP 266,032 published 4 May 1988, or via deoxynucloside H-phosphonate intermediates as described by Froeshler et al., Nucl. Acids, Res., 14:5399-5407 (1986)). Further methods include the polymerase chain reaction defined below and other autoprimer methods and oligonucleotide syntheses on solid supports. All of these methods are described in Engels et al., Agnew. Chem. Int. Ed. Engl., 28:716-734 (1989). These methods are used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation.

DNA is “purified” when the DNA is separated from non-nucleic acid impurities. The impurities may be polar, non-polar, ionic, etc.

A “transcription regulatory element” will contain one or more of the following components: an enhancer element, a promoter, an operator sequence, a repressor gene, and a transcription termination sequence. These components are well known in the art, e.g., U.S. Pat. No. 5,667,780.

A “transformant” is a cell that has taken up and maintained DNA as evidenced by the expression of a phenotype associated with the DNA (e.g., antibiotic resistance conferred by a protein encoded by the DNA).

“Transformation” means a process whereby a cell takes up DNA and becomes a “transformant”. The DNA uptake may be permanent or transient.

A “variant” or “mutant” of a starting or reference polypeptide (for e.g., a source antibody or its variable domain(s)), such as a fusion protein (polypeptide) or a heterologous polypeptide (heterologous to a phage), is a polypeptide that 1) has an amino acid sequence different from that of the starting or reference polypeptide and 2) was derived from the starting or reference polypeptide through either natural or artificial (manmade) mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source antibody/antigen binding fragment or polypeptide) would be a variant polypeptide with respect to a source antibody or antigen binding fragment or polypeptide. Thus, a variant VH refers to a VH comprising a variant sequence with respect to a starting or reference polypeptide sequence (such as that of a source antibody or antigen binding fragment or polypeptide). A variant amino acid, in this context, refers to an amino acid different from the amino acid at the corresponding position in a starting or reference polypeptide sequence (such as that of a source antibody or antigen binding fragment or polypeptide). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites. Methods for generating amino acid sequence variants of polypeptides are described in U.S. Pat. No. 5,534,615, expressly incorporated herein by reference.

A “wild type” or “reference” sequence or the sequence of a “wild type” or “reference” protein/polypeptide, such as a coat protein, a CDR, or a variable domain of a source antibody, is the reference sequence from which variant polypeptides are derived through the introduction of mutations. In general, the “wild type” sequence for a given protein is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

As used herein “Vh3” refers to a subgroup of antibody variable domains. The sequences of known antibody variable domains have been analyzed for sequence identity and divided into groups. Antibody heavy chain variable domains in subgroup III are known to have a Protein A binding site.

A “plurality” or “population” of a substance, such as a polypeptide or polynucleotide of the invention, as used herein, generally refers to a collection of two or more types or kinds of the substance. There are two or more types or kinds of a substance if two or more of the substances differ from each other with respect to a particular characteristic, such as the variant amino acid found at a particular amino acid position. In a nonlimiting example, there is a plurality or population of polynucleotides of the invention if there are two or more polynucleotides of the invention that are substantially the same, preferably identical, in sequence except for one or more variant amino acids at particular CDR amino acid positions.

B. Modes of the Invention

A diverse library of isolated antibody variable domains is useful to identify novel antigen binding molecules having high affinity. Generating a library with antibody variable domains that are not only highly diverse, but are also structurally stable permits the isolation of high affinity binding antibody variable domains from the library that can more readily be produced in cell culture on a large scale. The present invention is based on the showing that the folding stability of an isolated heavy chain antibody variable domain can be enhanced by enhancing the hydrophilicity of those portions of the heavy chain antibody variable domain that typically interact with the light chain antibody variable domain when in the context of an intact antibody. In one aspect, VH residues that typically interact with the VL domain include amino acid positions 37, 39, 44, 45, 47, 91, and 103. In certain embodiments, one or more of the VH residues that typically interact with the VL domain are increased in hydrophilicity while one or more other such residues are maintained or decreased in hydrophilicity. It will be understood that by increasing the hydrophobicity of one or more residues that typically interact with the VL domain, the hydrophilicity of one or more other such residues, or the overall hydrophilicity of the portion of the VH domain that interacts with a VL domain may be increased. In certain embodiments, such modifications improve stability of the overall isolated heavy chain antibody variable domain while still permitting full and unbiased diversification at one or more of the three heavy chain complementarity determining regions.

It will be appreciated by one of ordinary skill in the art that yield, aggregation tendency, and thermal stability, while indicators of the overall folding stability of the protein, may be separately useful. Thus, as a nonlimiting example, a mutant VH domain with improved yield and thermal stability but also increased aggregation tendency relative to a wild-type VH domain may still be useful for applications in which increased aggregation is not problematic. Similarly, in another nonlimiting example, a mutant VH domain with decreased yield but decreased aggregation tendency and increased thermal stability relative to a wild-type VH domain may still be useful for applications in which large quantities of protein are not required, or where it is feasible to perform multiple rounds of protein isolation.

In one embodiment, modifications of the amino acid at position 37 of the isolated VH domain are provided. In one aspect, the amino acid at position 37 is a hydrophobic amino acid. In one such aspect, the amino acid at position 37 is selected from tryptophan, phenylalanine, and tyrosine. In another embodiment, modifications of the amino acid at position 39 of the isolated VH domain is provided. In one aspect, the amino acid at position 39 is a hydrophilic amino acid. In one aspect, the amino acid at position 39 is selected from arginine and aspartic acid. In another embodiment, modifications of the amino acid at position 45 of the isolated VH domain are provided. In one aspect, the amino acid at position 45 is a hydrophobic amino acid. In one such aspect, the amino acid at position 45 is selected from tryptophan, phenylalanine, and tyrosine. In another aspect, the amino acid at position 45 is a hydrophilic amino acid. In one such aspect, the amino acid at position 45 is glutamic acid. In another embodiment, modifications of the amino acid at position 47 of the isolated VH domain are provided. In one aspect, the amino acid at position 47 is selected from alanine, glutamic acid, leucine, threonine, and valine. In another embodiment, an isolated VH domain comprises two or more modifications at amino acid positions 37, 39, 44, 45, 47, 91, and/or 103. In another embodiment, an isolated VH domain comprises three or more modifications at amino acid positions 37, 47, 50, and/or 103. In another embodiment, an isolated VH domain comprises four or more modifications at amino acid positions 37, 47, 50, and 103. In another embodiment, the above mutations are made in the context of SEQ ID NO: 1. In another embodiment, the above mutations are made in the context of SEQ ID NO: 2.

The invention also provides further modifications that may be made within the framework regions of the isolated heavy chain variable domain to further increase the folding stability of the polypeptide. It was known that the stability of an isolated heavy chain antibody variable domain was enhanced when the histidine at amino acid position 35 was modified to glycine (Jespers et al., J. Mol. Biol. (2004) 337: 893-903). Applicants herein also identify other structural modifications that improve isolated heavy chain antibody binding domain stability.

In one aspect, modifications of the histidine at amino acid position 35 of the isolated VH domain to an amino acid other than glycine are provided. In one such aspect, the histidine at amino acid position 35 is modified to a serine. In another such aspect, the histidine at amino acid position 35 is modified to an alanine. In another such aspect, the histidine at amino acid position 35 is modified to an aspartic acid. In another aspect, the histidine at amino acid position 35 is modified to glycine, and one or more additional mutations are made in VH such that the isolated VH domain has increased folding stability relative to a VH domain with a single mutation comprising H35G.

In another aspect, modifications of the amino acid at position 50 of the isolated VH domain are provided. In one such aspect, the amino acid at position 50 is modified to a hydrophilic amino acid. In another such aspect, the amino acid at position 50 is modified to a serine. In another such aspect, the amino acid at position 50 is modified to a glycine. In another such aspect, the amino acid at position 50 is modified to an arginine. In another embodiment, an isolated VH domain comprises modifications at both amino acid positions 35 and 50.

In another embodiment, an isolated VH domain comprises two or more modifications at amino acid positions 35, 37, 39, 44, 45, 47, 50, 91, and/or 103. In one example, the invention provides a novel combination of modifications at amino acid positions 35 and 47 of an isolated VH domain. In one aspect, the amino acid at position 35 is serine, and the amino acid at position 47 is selected from phenylalanine and glutamic acid. In another aspect, the amino acid at position 35 is glycine and the amino acid at position 47 is methionine. In another aspect, the amino acid at position 35 is alanine and the amino acid at position 47 is selected from tryptophan and methionine.

In another embodiment, an isolated VH domain comprises three or more modifications at amino acid positions 37, 47, 50, and 103. In another embodiment, an isolated VH domain comprises

The polypeptides of the invention find uses in research and medicine. The polypeptides described herein are isolated VH domains with enhanced folding stability relative to wild-type VH domains, which can be specific for one or more target antigens. Such VH domains can be used, for example, as diagnostic reagents for the presence of the one or more target antigens. It may be preferred to use the VH domains of the invention over a wild-type VH domain specific for the one or more target antigens because the increased folding stability of the VH domains of the invention may permit them to retain activity for longer periods of time and under harsher conditions than a wild-type VH domain might, thereby making them desirable reagents for use in, e.g., diagnostic kits. For the same reason, the VH domains of the invention may be preferred for the construction of, e.g., affinity chromatography columns for the purification of the one or more target antigens. Increased folding stability of the VH domains of the invention should increase their ability to withstand denaturation over wild-type VH domains, and thus permit more stringent purification and selection conditions than a wild-type VH domain might allow. Enhanced folding stability also improves the yield of a protein when prepared, e.g., from cellular culture, due to less presence of misfolded or unfolded species that would typically be degraded by cellular proteases.

The polypeptides of the invention also find uses in medicine. Isolated VH domains may themselves serve as therapeutics, binding to one or more target antigens in vivo, or may be fused to one or more therapeutic molecules and serve a targeting function. In either case, enhanced stability of the VH domain/fusion protein should enhance its efficacy, potentially decrease the amount of the VH domain/fusion protein needed to be administered to achieve a given therapeutic outcome, thereby potentially decreasing nonspecific interactions with non-target antigens.

In another embodiment, the present invention provides methods of significantly increasing the folding stability of an isolated heavy chain antibody binding domain without compromising the ability of the domain to be diversified for one or more specific target antigens. The invention also provides isolated heavy chain antibody binding domains particularly well suited as VH domain scaffolds for display and selection of VH domains specific for one or more target antigens.

In another embodiment, both FR and CDR amino acid positions in the VH domain are modified such that the VH domain has increased folding stability relative to a wild-type VH domain. The modified CDR amino acid positions may be in CDRH1, CDRH2, and/or CDRH3, and mixtures thereof. In one aspect, the VH domain is an isolated VH domain. In another aspect, the VH domain is associated with a VL domain. In such an aspect, the VL domain may also include modifications at one or more amino acid positions, e.g., at CDRL1, CDRL2, CDRL3, and/or VL FR residues.

CDR amino acid positions can each be mutated using a non-random codon set encoding the commonly occurring amino acids at each amino acid position. In some embodiments, when a solvent accessible and highly diverse amino acid position in a CDR region is to be mutated, a codon set is selected that encodes preferably at least about 50%, preferably at least about 60%, preferably at least about 70%, preferably at least about 80%, preferably at least about 90%, preferably all the target amino acids (as defined above) for that position. In some embodiments, when a solvent accessible and highly diverse amino acid position in a CDR region is to be mutated, a codon set is selected that encodes preferably from about 50% to about 100%, preferably from about 60% to about 95%, preferably from at least about 70% to about 90%, preferably from about 75% to about 90% of all the target amino acids (as defined above) for that position.

In another aspect of the invention, the residues of one or more CDR regions of a polypeptide of the invention are those of naturally occurring antibodies or antigen-binding fragments thereof, or can be those from known antibodies or antigen-binding fragments thereof that bind to a particular antigen whether naturally occurring or synthetic. In some embodiments, the CDR regions may be randomized at each amino acid position. It will be understood by those of skill in the art that antigen binding molecules of the invention may require further optimization of antigen binding affinity using standard methods. In one embodiment, one or more CDR region amino acid sequences are taken from a camelid antibody amino acid sequence. In another embodiment, one or more CDR region amino acid sequences are taken from the closest human germline sequence corresponding to a camelid antibody amino acid sequence.

The diversity of the library or population of the antibody variable domains is designed to maximize diversity while optimizing of the structure of the antibody variable domain to provide for increased ability to isolate high affinity antibodies having improved folding stability relative to a wild-type VH domain. The number of positions mutated in the antibody variable domain is minimized or specifically targeted. In some cases, the variant amino acids at each position are designed to include the commonly occurring amino acids at each position, while preferably (where possible) excluding uncommonly occurring amino acids. In other cases, structural amino acid positions are identified and diversity is minimized at those positions to ensure a well folded polypeptide. In certain embodiments, a single antibody or antigen binding polypeptide including at least one CDR is used as the source polypeptide.

The invention provides methods of generating VH domains having improved folding stability relative to a wild-type VH domain while still permitting diversification at one or more CDR amino acid positions such that one or more VH domains with improved folding stability with specificity for a particular target antigen can be identified. The invention also provides methods for designing a VH domain having improved folding stability relative to a wild-type VH domain while still permitting diversification at one or more CDR amino acid positions. The invention also provides methods of increasing the stability of an isolated heavy chain antibody variable domain, comprising increasing the hydrophilicity of one or more amino acids of the heavy chain antibody variable domain known to interact with the VL domain.

In one aspect, the VH domain can be modified at one or more amino acid positions known to interact with VL. In one such aspect, the hydrophilicity of the portion of the VH domain known to interact with the VL is increased. In another such aspect, the hydrophobicity of the portion of the VH domain known to interact with the VL is decreased. In one such aspect, the one or more amino acid positions in the VH domain known to interact with the VL are selected from amino acid positions 37, 39, 44, 45, 47, 91, and 103.

It is surprising that a library of antibody variable domains with high affinity antigen binders having diversity in sequences and size while also having increased folding stability can be generated using a single source polypeptide as a template and targeting diversity to particular positions using particular amino acid substitutions.

1. Generating Diversity in Isolated VH

High quality polypeptide libraries of antibody variable domains may be generated by diversifying one or more heavy chain antibody variable domain (VH) framework amino acid positions, and optionally one or more CDRs, of a source antibody or antibody fragment. The polypeptide libraries comprise a plurality of variant polypeptides having at least one amino acid modification at a VH framework residue that increases the folding stability of the VH. In certain embodiments, the framework and/or CDR modifications are designed to provide for amino acid sequence diversity at certain positions while maximizing structural stability of the VH domain.

The diversity of the library or population of the heavy chain antibody variable domains is designed to maximize diversity while enhancing structural stability of the heavy chain antibody variable domain to provide for increased ability to isolate VH having high affinity for one or more target antigens. The number of positions mutated in the heavy chain antibody variable domain framework region is minimized or specifically targeted. In some embodiments, structural amino acid positions are identified and diversity is minimized at those positions to ensure a well-folded polypeptide. Preferably, a single antibody or antigen binding polypeptide including at least one CDR is used as the source polypeptide.

The source polypeptide may be any antibody, antibody fragment, or antibody variable domain whether naturally occurring or synthetic. A polypeptide or source antibody variable domain can include an antibody, antibody variable domain, antigen binding fragment or polypeptide thereof, a monobody, VHH, a monobody or antibody variable domain obtained from a naïve or synthetic library, camelid antibodies, naturally occurring antibody or monobody, synthetic antibody or monobody, recombinant antibody or monobody, humanized antibody or monobody, germline derived antibody or monobody, chimeric antibody or monobody, and affinity matured antibody or monobody. In one embodiment, the polypeptide is an antibody variable domain that is a member of the Vh3 subgroup.

Source antibody variable domains include, but are not limited to, antibody variable domains previously used to generate phage display libraries, such as VHH-RIG, VHH-VLK, VHH-LLR, and VHH-RLV (Bond et al., 2003, J. Mol. Biol., 332:643-655), and humanized antibodies or antibody fragments, such as mAbs 4D5, 2C4, and A4.6.1. Table A shows the amino acid sequence of CDR3 in the source VHH-RIG, VHH-VLK, VHH-LLR, and VHH-RLV scaffolds. In an embodiment, the library is generated using the heavy chain variable domain (VHH) of a monobody as a source antibody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

TABLE A

| | SEQ | | | | | | | | | | | | | | | | | |
|---|---|---|---|---|---|---|---|---|---|---|---|---|---|---|---|---|---|---|
| VHH | ID | CDRH3 Position | | | | | | | | | | | | | | | | |
| Scaffold | NO: | 96 | 97 | 98 | 99 | 100 | 100a | 100b | 100c | 100d | 100e | 100f | 100g | 100h | 100i | 100j | 100k | 100l |
| RIG | 3 | R | I | G | R | S | V | F | N | L | R | R | E | S | W | V | T | W |
| LLR | 4 | L | L | R | R | G | V | N | A | T | P | N | W | F | G | L | V | G |
| VLK | 5 | V | L | K | R | R | G | S | S | V | A | I | F | T | R | V | Q | S |
| RLV | 6 | R | L | V | N | G | L | S | G | L | V | S | W | E | M | P | L | A |

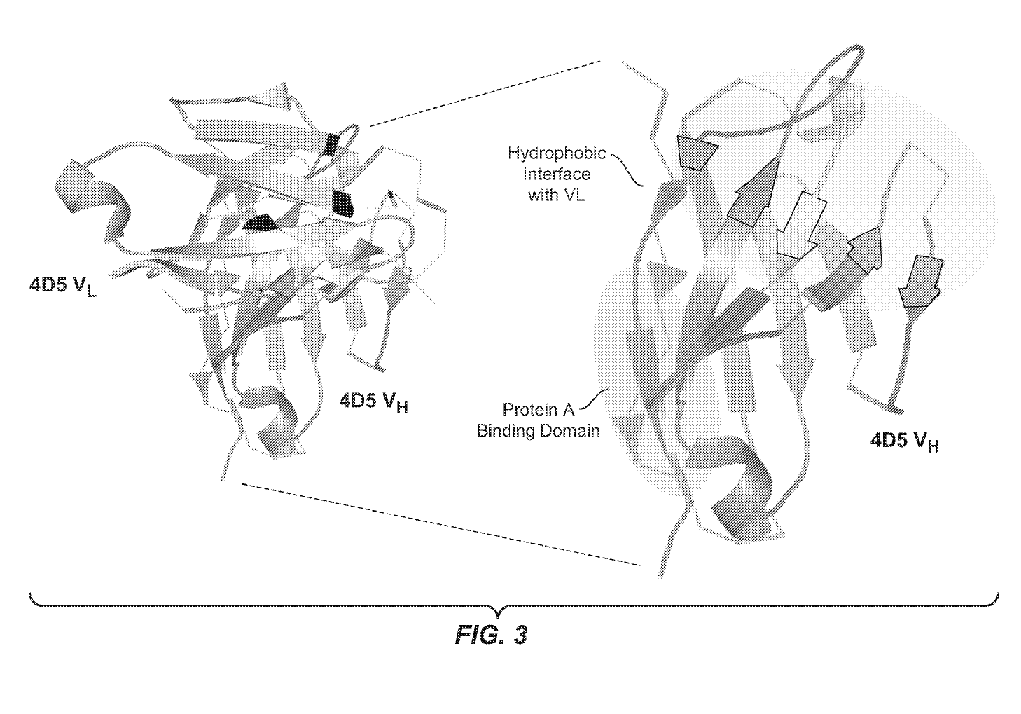

One criterion for generating diversity in the polypeptide library is selecting regions of the VH domain that normally interact with a VL domain (“VL-interacting” residues). Such regions typically have significant hydrophobic character, and in the absence of a VL domain, lead to aggregation and decreased stability of the isolated VH domain. One way of determining whether a given amino acid position is part of a VL-interacting region on a VH domain is to examine the three dimensional structure of the antibody variable domain, for example, for VL-interacting positions. If such information is available, amino acid positions that are in proximity to the antigen can also be determined. Three dimensional structure information of antibody variable domains are available for many antibodies or can be prepared using available molecular modeling programs. VL-interacting amino acid positions can be found in FR and/or at the edge of CDRs, and typically are exposed at the exterior of the protein (see, e.g., FIG. 3). Preferably, appropriate amino acid positions are identified using coordinates from a 3-dimensional model of an antibody, using a computer program such as the InsightII program (Accelrys, San Diego, Calif.). Such amino acid positions can also be determined using algorithms known in the art (e.g., Lee and Richards, J. Mol. Biol. 55, 379 (1971) and Connolly, J. Appl. Cryst. 16, 548 (1983)). Determination of VL-interacting positions can be performed using software suitable for protein modeling and 3-dimensional structural information obtained from an antibody. Software that can be utilized for these purposes includes SYBYL Biopolymer Module software (Tripos Associates). Generally, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios ((1994) “ARVOMOL/CONTOUR: molecular surface areas and volumes on Personal Computers”, Comput. Chem. 18(4): 377-386; and “Variations of Surface Areas and Volumes in Distinct Molecular Surfaces of Biomolecules.” J. Mol. Model. (1995), 1: 46-53). The location of amino acid positions involved in VL interaction may vary in different antibody variable domains, but typically involve at least one or a portion of an FR and occasionally at least one portion of a CDR.

In some instances, selection of VL-interacting residues is further refined by choosing VL-interacting residues that collectively form a minimum contiguous patch when the reference polypeptide or source antibody is in its 3-D folded structure. A compact (minimum) contiguous patch may comprise a portion of the FR and only a subset of the full range of CDRs, for example, CDRH1/H2/H3. VL-interacting residues that do not contribute to formation of such a patch may optionally be excluded from diversification. Refinement of selection by this criterion permits the practitioner to minimize, as desired, the number of residues to be diversified. This selection criterion may also be used, where desired, to choose residues to be diversified that may not necessarily be deemed to be VL-interacting. For example, a residue that is not deemed VL-interacting, but forms a contiguous patch in the 3-D folded structure with other residues that are deemed VL-interacting may be selected for diversification. Selection of such residues would be evident to one skilled in the art, and its appropriateness can also be determined empirically and according to the needs and desires of the skilled practitioner.

VH framework region and CDR diversity may be limited at structural amino acid positions. A structural amino acid position refers to an amino acid position in a VH framework region or CDR that contributes to the stability of the structure of the polypeptide such that the polypeptide retains at least one biological function such as specifically binding to a molecule such as an antigen. In certain embodiments, such a polypeptide specifically binds to a target molecule that binds to folded polypeptide and does not bind to unfolded polypeptide, such as Protein A. Structural amino acid positions of a VH framework region or CDR are identified as amino acid positions less tolerant to amino acid substitutions without negatively affecting the structural stability of the polypeptide. Typically, CDR regions do not contain structural amino acid positions, but upon modification of one or more FR amino acid positions, one or more CDR amino acid positions may become a structural amino acid position.

Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the VH framework region or CDR. An amino acid position is important to maintaining the structure of the polypeptide if a wild type amino acid is replaced with a scanning amino acid in an amino acid position in a VH framework region and the resulting variant exhibits poor binding to a target molecule that binds to folded polypeptide. A structural amino acid position is a position in which the ratio of sequences with the wild type amino acid at a position to sequences with the scanning amino acid at that position is at least about 3 to 1, 5 to 1, 8 to 1, or about 10 to 1 or greater.

Alternatively, structural amino acid positions and nonstructural amino acid positions in a VH framework region or CDR can be determined by calculating the Shannon entropy at each selected VL-interacting position. Antibody variable domains with each selected amino acid position (whether a CDR or FR position) are randomized and selected for stability by binding to a molecule that binds properly folded antibody variable domains, such as protein A. Binders are isolated and sequenced and the sequences are compared to a database of antibody variable domain sequences from an appropriate species (e.g., human and/or mouse). The per residue variation in the randomized population can be estimated using the Shannon entropy calculation, with a value close to about 0 indicating that the amino acid in that position is conserved and values close to about 4.23 representing an amino acid position that is tolerant to substitution with all 20 amino acids. A structural amino acid position is identified as a position that has a Shannon entropy value of about 2 or less.

In a further embodiment, structural amino acid positions can be determined based on weighted hydrophobicity for example, according to the method of Kyte and Doolittle. Structural amino acid positions and nonstructural amino acid positions in a VH framework region or CDR can be determined by calculating the weighted hydrophobicity at each selected VL-interacting position. Antibody variable domains with each selected amino acid position (whether a CDR or FR position) are randomized and selected for stability by binding to a molecule that binds properly folded antibody variable domains, such as protein A. Binders are isolated and sequenced. The weighted hydrophobicity at each position is calculated and those positions that have a weighted hydrophobicity of greater than the average hydrophobicity for any amino acid are selected as structural amino acid positions. The weighted hydrophobicity is in one embodiment greater than −0.5, and in another embodiment greater than 0 or 1.

Once the structural amino acid positions are identified, diversity is minimized or limited at those positions in order to provide a library with a diverse VH framework region while minimizing structural perturbations. The number of amino acids that are substituted at a structural amino acid position is no more than about 1 to 7, about 1 to 4 or about 1 to 2 amino acids. In some embodiments, a variant amino acid at a structural amino acid position is encoded by one or more nonrandom codon sets. The nonrandom codon sets encode multiple amino acids for a particular position, for example, about 1 to 7, about 1 to 4 amino acids or about 1 to 2 amino acids.

In one embodiment, the amino acids that are substituted at structural positions are those that are found at that position in a randomly generated VH framework region population at a frequency at least one standard deviation above the average frequency for any amino acid at the position. In one embodiment, the frequency is at least 60% or greater than the average frequency for any amino acid at that position, more preferably the frequency is at least one standard deviation (as determined using standard statistical methods) greater than the average frequency for any amino acid at that position. In another embodiment, the set of amino acids selected for substitution at the structural amino acid positions comprise, consist essentially of, or consist of the 6 amino acids that occur most commonly at that positions as determined by calculating the fractional occurrence of each amino acid at that positions using standard methods. In some embodiments, the structural amino acids are preferably a hydrophobic amino acid or a cysteine as these amino acid positions are more likely to be buried and point into the core.

A variant VH framework region is typically positioned between the VH CDRs. The randomized VH framework regions may contain one or more non-structural amino acid positions that have a variant amino acid. Non-structural amino acid positions may vary in sequence and length. The non-structural amino acid positions can be substituted randomly with any of the naturally occurring amino acids or with selected amino acids. In some embodiments, one or more non-structural positions can have a variant amino acid encoded by a random codon set or a nonrandom codon. The nonrandom codon set preferably encodes at least a subset of the commonly occurring amino acids at those positions while minimizing nontarget sequences such as cysteine and stop codons. Examples of nonrandom codon sets include but are not limited to DVK, XYZ, and NVT. Examples of random codon sets include but are not limited to NNS and NNK.

In another embodiment, VH diversity is generated using the codon set NNS. NNS and NNK encode the same amino acid group. However, there can be individual preferences for one codon set or the other, depending on the various factors known in the art, such as efficiency of coupling in oligonucleotide synthesis chemistry.

In some embodiments, the practitioner of methods of the invention may wish to modify the amount/proportions of individual nucleotides (G, A, T, C) for a codon set, such as the N nucleotide in a codon set such as in NNS. This is illustratively represented as XYZ codons. This can be achieved by, for example, doping different amounts of the nucleotides within a codon set instead of using a straight, equal proportion of the nucleotides for the N in the codon set. Such modifications can be useful for various purposes depending on the circumstances and desire of the practitioner. For example, such modifications can be made to more closely reflect the amino acid bias as seen in a natural diversity profile, such as the profile of the VH domain.

In some embodiments, non-structural amino acid position regions can also vary in length. For example, FR3 of naturally occurring heavy chains can have lengths ranging from 29 amino acids up to 41 amino acids depending on whether the CDRs are defined according to Kabat or Chothia. The contiguous loop of nonstructural amino acids can vary from about 1 to 20 amino acids, more preferably 6 to 15 amino acids and more preferably about 6 to 10 amino acids.

When the polypeptide is an antibody heavy chain variable domain, diversity at other selected framework region residues aside from the structural amino acids may also be limited in order to preserve structural stability of the polypeptide. The diversity in framework regions can also be limited at those positions that form the light chain interface. In some embodiments, the positions that form the light chain interface are diversified with residues encoding hydrophilic amino acids. The amino acid positions that are found at the light chain interface in the VHH of camelid monobodies include amino acid position 37, amino acid position 45, amino acid position 47 and amino acid position 91. Heavy chain interface residues are those residues that are found on the heavy chain but have at least one side chain atom that is within 6 angstroms of the light chain. The amino acid positions in the heavy chain that are found at the light chain interface in human heavy chain variable domains include positions 37, 39, 44, 45, 47, 91, and 103.

Once the libraries with diversified VH framework regions are prepared they can be selected and/or screened for binding to one or more target antigens. In addition, the libraries may be selected for improved binding affinity to particular target antigen. The target antigens may be any type of antigenic molecule but preferably are a therapeutic target molecule including, but not limited to, interferons, VEGF, Her-2, cytokines, and growth factors. In certain embodiments, the target antigen may be one or more of the following: growth hormone, bovine growth hormone, insulin like growth factors, human growth hormone including n-methionyl human growth hormone, parathyroid hormone, thyroxine, insulin, proinsulin, amylin, relaxin, prorelaxin, glycoprotein hormones such as follicle stimulating hormone (FSH), leutinizing hormone (LH), hematopoietic growth factor, fibroblast growth factor, prolactin, placental lactogen, tumor necrosis factors, mullerian inhibiting substance, hepatocyte growth factor, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins, bone morphogenetic proteins, LIF,SCF,FLT-3 ligand and kit-ligand, or receptors for any of the foregoing.

Another aspect of the invention includes compositions of the polypeptides, fusion proteins or libraries of the invention. Compositions comprise a polypeptide, a fusion protein, or a population of polypeptides or fusion proteins in combination with a physiologically acceptable carrier.

2. Variant VHs

As discussed above, randomized VHs can generate polypeptide libraries that bind to a variety of target molecules, including antigens. These randomized VHs can be incorporated into other antibody molecules or used to form a single chain mini-antibody with an antigen binding domain comprising a heavy chain variable domain but lacking a light chain. Within the VH, amino acid positions that are primarily structural have limited diversity and other amino acids that do not contribute significantly to structural stability may be varied both in length and sequence diversity.

Polypeptides comprising a VH domain described herein are also provided by the invention. Polypeptides comprising a VH domain include, but are not limited to, a camelid monobody, VHH, camelized antibodies, antibody or monobody variable domain obtained from a naïve or synthetic library, naturally occurring antibody or monobody, recombinant antibody or monobody, humanized antibody or monobody, germline derived antibody or monobody, chimeric antibody or monobody, and affinity matured antibody or monobody. It will be appreciated by those of ordinary skill in the art that amino acid modifications that enhance folding stability of an isolated VH domain may be more or less effective for that purpose when the VH domain is part of a larger molecule, e.g., an antibody or a fusion protein. When the intent is for the VH domain to be used in the context of a larger molecule, e.g., a fusion protein, then randomization of one or more nonstructural amino acid positions suspected or known to be VL-interacting may be performed in the context of the larger molecule rather than in the VH domain alone.

A number of different combinations of structural amino acid positions and nonstructural amino acid positions can be designed in a VH template. In some variations of the aforementioned embodiments, and as described in the examples herein, non-structural amino acid positions can also vary in length.

3. Diversity in CDR Regions

The library or population of the heavy chain antibody variable domains is designed to maximize diversity while also maximizing structural stability of the heavy chain antibody variable domain to provide for increased ability to isolate high affinity binders. The number of positions mutated in the heavy chain antibody variable domain framework region is minimized or specifically targeted. In some embodiments, structural amino acid positions are identified and diversity is minimized at those positions to ensure a well-folded polypeptide. The positions mutated or changed include positions in FR and/or one or more of the CDR regions and combinations thereof.

The source polypeptide may be any antibody, antibody fragment, or antibody variable domain whether naturally occurring or synthetic. A polypeptide or source antibody variable domain can include an antibody, antibody variable domain, antigen binding fragment or polypeptide thereof, a monobody, VHH, a monobody or antibody variable domain obtained from a naïve or synthetic library, camelid antibodies, naturally occurring antibody or monobody, synthetic antibody or monobody, recombinant antibody or monobody, humanized antibody or monobody, germline derived antibody or monobody, chimeric antibody or monobody, and affinity matured antibody or monobody. In one embodiment, the polypeptide is a heavy chain antibody variable domain that is a member of the Vh3 subgroup.

Source antibody variable domains include, but are not limited to, antibody variable domains previously used to generate phage display libraries, such as VHH-RIG, VHH-VLK, VHH-LLR, and VHH-RLV (Bond et al., 2003, J. Mol. Biol., 332:643-655), and humanized antibodies or antibody fragments, such as mAbs 4D5, 2C4, and A4.6. 1. In one embodiment, the library is generated using the heavy chain variable domain (VHH) of a monobody. The small size and simplicity make monobodies attractive scaffolds for peptidomimetic and small molecule design, as reagents for high throughput protein analysis, or as potential therapeutic agents. The diversified VHH domains are useful, inter alia, in the design of enzyme inhibitors, novel antigen binding molecules, modular binding units in bispecific or intracellular antibodies, as binding reagents in protein arrays, and as scaffolds for presenting constrained peptide libraries.

One criterion for generating diversity in the polypeptide library is selecting amino acid positions that (1) interact with a VL domain and/or (2) interact with a target antigen. Three dimensional structure information of antibody variable domains are available for many antibodies or can be prepared using available molecular modeling programs. VL-interacting accessible amino acid positions can be found in FR and CDRs. In certain embodiments, VL-interacting positions are determined using coordinates from a 3-dimensional model of an antibody, using a computer program such as the InsightII program (Accelrys, San Diego, Calif.). VL-interacting amino acid positions can also be determined using algorithms known in the art (e.g., Lee and Richards, J. Mol. Biol. 55, 379 (1971) and Connolly, J. Appl. Cryst. 16, 548 (1983)). Determination of such VL-interacting positions can be performed using software suitable for protein modeling and 3-dimensional structural information obtained from an antibody. Software that can be utilized for these purposes includes SYBYL Biopolymer Module software (Tripos Associates). Generally, where an algorithm (program) requires a user input size parameter, the “size” of a probe which is used in the calculation is set at about 1.4 Angstrom or smaller in radius. In addition, determination of VL-interacting regions and area methods using software for personal computers has been described by Pacios ((1994) “ARVOMOL/CONTOUR: molecular surface areas and volumes on Personal Computers”, Comput. Chem. 18(4): 377-386; and “Variations of Surface Areas and Volumes in Distinct Molecular Surfaces of Biomolecules.” J. Mol. Model. (1995), 1: 46-53). The location of VH amino acid positions involved in a VL-interaction may vary in different antibody variable domains, but typically involve at least one or a portion of a FR and occasionally a portion of a CDR region.

In some instances, selection of VL-interacting residues is further refined by choosing VL-interacting residues that collectively form a minimum contiguous patch when the reference polypeptide or source antibody is in its 3-D folded structure. A compact (minimum) contiguous patch may comprise a portion of the FR and only a subset of the full range of CDRs, for example, CDRH1/H2/H3. VL-interacting residues that do not contribute to formation of such a patch may optionally be excluded from diversification. Refinement of selection by this criterion permits the practitioner to minimize, as desired, the number of residues to be diversified. This selection criterion may also be used, where desired, to choose residues to be diversified that may not necessarily be deemed VL-interacting. For example, a residue that is not deemed VL-interacting, but that forms a contiguous patch in the 3-D folded structure with other residues that are deemed VL-interacting may be selected for diversification. Selection of such residues would be evident to one skilled in the art, and its appropriateness can also be determined empirically and according to the needs and desires of the skilled practitioner.

CDR diversity may be limited at structural amino acid positions. A structural amino acid position refers to an amino acid position in a CDR of a polypeptide that contributes to the stability of the structure of the polypeptide such that the polypeptide retains at least one biological function such as specifically binding to a molecule such as an antigen, or specifically binds to a target molecule that binds to folded polypeptide and does not bind to unfolded polypeptide, such as Protein A. Structural amino acid positions of a CDR are identified as amino acid positions less tolerant to amino acid substitutions without affecting the structural stability of the polypeptide, as described above.

Amino acid positions less tolerant to amino acid substitutions can be identified using a method such as alanine scanning mutagenesis or shotgun scanning as described in WO 01/44463 and analyzing the effect of loss of the wild type amino acid on structural stability at positions in the CDR. An amino acid position is important to maintaining the structure of the polypeptide if a wild type amino acid is replaced with a scanning amino acid in an amino acid position in a CDR and the resulting variant exhibits poor binding to a target molecule that binds to folded polypeptide. A structural amino acid position is a position in which the ratio of sequences with the wild type amino acid at a position to sequences with the scanning amino acid at that position is at least about 3 to 1, 5 to 1, 8 to 1, or about 10 to 1 or greater.

Alternatively, structural amino acid positions and nonstructural amino acid positions in a VH framework region or CDR can be determined by calculating the Shannon entropy at each selected VL-interacting position. Antibody variable domains with each selected amino acid position (whether a CDR or FR position) are randomized and selected for stability by binding to a molecule that binds properly folded antibody variable domains, such as protein A. Binders are isolated and sequenced and the sequences are compared to a database of antibody variable domain sequences from an appropriate species (e.g., human and/or mouse). The per residue variation in the randomized population can be estimated using the Shannon entropy calculation, with a value close to about 0 indicating that the amino acid in that position is conserved and values close to about 4.23 representing an amino acid position that is tolerant to substitution with all 20 amino acids. A structural amino acid position is identified as a position that has a Shannon entropy value of about 2 or less.

In a further embodiment, structural amino acid positions can be determined based on weighted hydrophobicity for example, according to the method of Kyte and Doolittle. Structural amino acid positions and nonstructural amino acid positions in a VH framework region or CDR can be determined by calculating the weighted hydrophobicity at each selected VL-interacting position. Antibody variable domains with each selected amino acid position (whether a CDR or FR position) are randomized and selected for stability by binding to a molecule that binds properly folded antibody variable domains, such as protein A. Binders are isolated and sequenced. The weighted hydrophobicity at each position is calculated and those positions that have a weighted hydrophobicity of greater than the average hydrophobicity for any amino acid are selected as structural amino acid positions. The weighted hydrophobicity is in one embodiment greater than −0.5, and in another embodiment greater than 0 or 1.

In some embodiments, structural amino acid positions in a CDRH1 are selected or located near the N and C terminus of the CDRH1 allowing for a central portion that can be varied. The structural amino acid positions are selected as the boundaries for a CDRH1 loop of contiguous amino acids that can be varied randomly, if desired. The variant CDRH1 regions can have a N terminal flanking region in which some or all of the amino acid positions have limited diversity, a central portion comprising at least one or more non-structural amino acid position that can be varied in length and sequence, and C-terminal flanking sequence in which some or all amino acid positions have limited diversity.

Initially, a CDRH1 region can include amino acid positions as defined by Chothia including amino acid positions 26 to 32. Additional amino acid positions can also be randomized on either side of the amino acid positions in CDRH1 as defined by Chothia, typically 1 to 3 amino acids at the N and/or C terminal end. The N terminal flanking region, central portion, and C-terminal flanking region is determined by selecting the length of CDRH1, randomizing each position and identifying the structural amino acid positions at the N and C-terminal ends of the CDR to set the boundaries of the CDR. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more non-structural positions can vary from about 1 to 20 contiguous amino acids, and the C-terminal portion is at least about from 1 to 6 contiguous amino acids. The central portion of contiguous amino acids can comprise, consist essentially of or consist of about 9 to about 15 amino acids and more preferably about 9 to 12 amino acids.

In some embodiments, structural amino acid positions in a CDRH2 are located near the N terminus of the CDRH2 allowing for a portion of CDRH2 adjacent to the N terminal that can be varied. The variant CDRH2 regions can have a N terminal flanking region in which some or all of the amino acid positions have limited diversity, and a portion comprising at least one or more non-structural amino acid position that can be varied in length and sequence.

Initially, a CDRH2 region can include amino acid positions as defined by Chothia including amino acid positions 53 to 55. Additional amino acid positions can be randomized on either side of the amino acid positions in CDRH2 as defined by Chothia, typically 1 to 3 amino acids on the N and/or C terminus. The length of the N terminal flanking region, and randomized central portion is determined by selecting the length of CDRH2, randomizing each position and identifying the structural amino acid positions at the N terminal ends of the CDR. The length of the N terminal flanking sequence should be long enough to include at least one structural amino acid position. In some embodiments, the length of the N-terminal flanking region is at least about from 1 to 4 contiguous amino acids, and the randomized portion of one or more non-structural positions can vary from about 1 to 20 contiguous amino acids. The central portion of contiguous amino acids can comprise, consist essentially of or consist of about 5 to about 15 amino acids and more preferably about 5 to 12 amino acids.

In some embodiments, structural amino acid positions in a CDRH3 are located near the N and C terminus of the CDRH3 allowing for a central portion that can be varied. The variant CDRH3 regions can have a N terminal flanking region in which some or all of the amino acid positions have limited diversity, a central portion comprising at least one or more non-structural amino acid position that can be varied in length and sequence, and C-terminal flanking sequence in which some or all amino acid positions have limited diversity.

The length of the N terminal flanking region, central portion, and C-terminal flanking region is determined by selecting the length of CDRH3, randomizing each position and identifying the structural amino acid positions at the N and C-terminal ends of the CDRH3. The length of the N and C terminal flanking sequences should be long enough to include at least one structural amino acid position in each flanking sequence. In some embodiments, the length of the N-terminal flanking region is at least about from 1 to 4 contiguous amino acids, the central portion of one or more non-structural positions can vary from about 1 to 20 contiguous amino acids, and the C-terminal portion is at least about from 1 to 6 contiguous amino acids.

In one embodiment, the CDRH3 is about 17 amino acids long and a library comprising a variant CDRH3 is generated. The variant CDRH3 comprises, consists essentially of, at least one structural amino acid position selected from at least one or two N terminal amino acids and at least one of the last six C terminal amino acids. The central portion comprises 11 amino acids that can be randomized if desired.

In one embodiment, the CDRH3 is an amino acid loop corresponding to amino acid positions 96 to 101 in the heavy chain of a monobody. The structural amino acids positions comprise, consist essentially of or consist of the two N terminal amino acid positions corresponding to amino acid positions 96, and 97, respectively. Table B shows the positions of the insertion of a randomized loop of amino acids into CDRH3. (SEQ ID NO: 249)

TABLE B

C G A G X X X X X X X X X X X X X X X X X D
92 96 97 98 99 100 a b c d e f g h i j k l 101

The amino acids that are substituted at structural positions can be those that are found at that position in a randomly generated CDR population at a frequency at least one standard deviation above the average frequency for any amino acid at the position. In one embodiment, the frequency is at least 60% or greater than the average frequency for any amino acid at that position, more preferably the frequency is at least one standard deviation (as determined using standard statistical methods) greater than the average frequency for any amino acid at that position. In another embodiment, the set of amino acids selected for substitution at the structural amino acid positions comprise, consist essentially of, or consist of the 6 amino acids that occur most commonly at that position as determined by calculating the fractional occurrence of each amino acid at that position using standard methods. In some embodiments, the structural amino acids are preferably a hydrophobic amino acid or a cysteine as these amino acid positions are more likely to be buried and point into the core.

The variant CDR is typically positioned between at amino acid positions that are typical boundaries for CDR regions in naturally occurring antibody variable domains and may be inserted within a CDR in a source variable domain. Typically, when the variant CDR is inserted into a source or wild type antibody variable domain, the variant CDR replaces all or a part of the source or wild type CDR. The location of insertion of the CDR can be determined by comparing the location of CDRs in naturally occurring antibody variable domains. Depending on the site of insertion the numbering can change.

The randomized CDR may also contain one or more non-structural amino acid positions that have a variant amino acid. Non-structural amino acid positions may vary in sequence and length. In some embodiments, one or more non-structural amino acid positions are located in between the N terminal and C terminal flanking regions. The non-structural amino acid positions can be substituted randomly with any of the naturally occurring amino acids or with selected amino acids. In some embodiments, one or more non-structural positions can have a variant amino acid encoded by a random codon set or a nonrandom codon. The nonrandom codon set preferably encodes at least a subset of the commonly occurring amino acids at those positions while minimizing nontarget sequences such as cysteine and stop codons. Examples of nonrandom codon sets include but are not limited to DVK, XYZ, and NVT. Examples of random codon sets include but are not limited to NNS and NNK.

In another embodiment, CDR diversity is generated using the codon set NNS. NNS and NNK encode the same amino acid group. However, there can be individual preferences for one codon set or the other, depending on the various factors known in the art, such as efficiency of coupling in oligonucleotide synthesis chemistry.

In some embodiments, the practitioner of methods of the invention may wish to modify the amount/proportions of individual nucleotides (G, A, T, C) for a codon set, such as the N nucleotide in a codon set such as in NNS. This is illustratively represented as XYZ codons. This can be achieved by, for example, doping different amounts of the nucleotides within a codon set instead of using a straight, equal proportion of the nucleotides for the N in the codon set. Such modifications can be useful for various purposes depending on the circumstances and desire of the practitioner. For example, such modifications can be made to more closely reflect the amino acid bias as seen in a natural diversity profile, such as the profile of CDR.

Once the libraries with diversified CDR regions are prepared they can be selected and/or screened for binding one or more target antigens. In addition, the libraries may be selected for improved binding affinity to particular target antigen. The target antigens may include any type of antigenic molecule. In certain embodiments, the target antigens include therapeutic target molecules, including, but not limited to, interferons, VEGF, Her-2, cytokines, and growth factors. In certain embodiments, the target antigen may be one or more of the following: growth hormone, bovine growth hormone, insulin like growth factors, human growth hormone including n-methionyl human growth hormone, hepatocyte growth factor, parathyroid hormone, thyroxine, insulin, proinsulin, amylin, relaxin, prorelaxin, glycoprotein hormones such as follicle stimulating hormone (FSH), leutinizing hormone (LH), hemopoietic growth factor, fibroblast growth factor, prolactin, placental lactogen, tumor necrosis factors, mullerian inhibiting substance, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins, bone morphogenetic proteins, LIF, SCF, FLT-3 ligand and kit-ligand, and receptors for any of the foregoing.

Antibody variable domains with targeted diversity in one or more FRs can be combined with targeted diversity in one or more CDRs as well. A combination of regions may be diversified in order to provide for high affinity antigen binding molecules or to improve the affinity of a known antibody such as a humanized antibody.

4. Polypeptide Variant Construction

In some embodiments, amino acid sequence modification(s) of the polypeptides described herein are contemplated, e.g., to increase the folding stability of the polypeptides. Amino acid sequence variants of the antibody are prepared by introducing appropriate nucleotide changes into the nucleic acid encoding a polypeptide of the invention, or by peptide synthesis. Such modifications include, for example, deletions from, and/or insertions into and/or substitutions of, residues within the amino acid sequences of the polypeptide of the invention (e.g., an isolated VH domain). Any combination of deletion, insertion, and substitution can be made to arrive at the final construct, provided that the final construct possesses the desired characteristics. The amino acid alterations may be introduced in the subject polypeptide amino acid sequence at the time that sequence is made.

A useful method for identification of certain residues or regions of an antibody, antibody fragment, or VH domain that are preferred locations for mutagenesis is called “alanine scanning mutagenesis” as described by Cunningham and Wells (1989) Science, 244:1081-1085. In that methodology, a residue or group of target residues are identified (e.g., charged residues such as arg, asp, his, lys, and glu) and replaced by a neutral or negatively charged amino acid (e.g., alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated as described herein. Conservative substitutions are shown in Table C under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in the biological properties of the antibody, antibody fragment, or VH domain are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His(H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, into the remaining (non-conserved) sites. One type of substitutional variant involves substituting one or more CDR residues of a source antibody (e.g. a humanized or human antibody) for one or more CDR residues of a polypeptide of the invention. Generally, the resulting variant(s) selected for further development will have modified (e.g., improved) biological properties relative to the parent polypeptide from which they are generated. A convenient way for generating such substitutional variants involves affinity maturation using phage display. Briefly, several amino acid positions (e.g. 6-7 sites) are mutated to generate all possible amino acid substitutions at each site. The antibodies thus generated are displayed from filamentous phage particles as fusions to at least part of a phage coat protein (e.g., the gene III product of M13) packaged within each particle. The phage-displayed variants are then screened for their biological activity (e.g. binding affinity and/or folding stability) as herein disclosed. In order to identify candidate sites for modification, scanning mutagenesis (e.g., alanine scanning) can be performed to identify amino acid positions contributing significantly to antigen binding and/or folding stability. Alternatively, or additionally, it may be beneficial to analyze a crystal structure of the antigen-antibody complex to identify contact points between the antibody, antibody fragment, or VH domain and the antigen. Such contact residues and neighboring residues are candidates for substitution according to techniques known in the art, including those elaborated herein. Once such variants are generated, the panel of variants is subjected to screening using techniques known in the art, including those described herein, and antibodies, antibody fragments, or VH domains with superior properties in one or more relevant assays may be selected for further development.

5. Polynucleotides, Vectors, Host Cells, and Recombinant Methods

a. Oligonucleotides and Recombinant Methods

Nucleic acid molecules encoding amino acid sequence variants of the antibody, antibody fragment, or VH domain are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody, antibody fragment, or VH domain. For example, libraries can be created by targeting VL accessible amino acid positions in VH, and optionally in one or more CDRs, for amino acid substitution with variant amino acids using the Kunkel method. See, for e.g., Kunkel et al., Methods Enzymol. (1987), 154:367-382 and the examples herein. Generation of randomized sequences is also described below in the Examples.

The sequence of oligonucleotides includes one or more of the designed codon sets for a particular position in a CDR or FR region of a polypeptide of the invention. A codon set is a set of different nucleotide triplet sequences used to encode desired variant amino acids. Codon sets can be represented using symbols to designate particular nucleotides or equimolar mixtures of nucleotides as shown in below according to the IUB code.

IUB CODES

G Guanine

A Adenine

T Thymine

C Cytosine

R (A or G)

Y (C or T)

M (A or C)

K (G or T)

S (C or G)

W (A or T)

H (A or C or T)

B (C or G or T)

V (A or C or G)

D (A or G or T)

N (A or C or G or T)

For example, in the codon set DVK, D can be nucleotides A or G or T; V can be A or G or C; and K can be G or T. This codon set can present 18 different codons and can encode amino acids Ala, Trp, Tyr, Lys, Thr, Asn, Lys, Ser, Arg, Asp, Glu, Gly, and Cys.

Oligonucleotide or primer sets can be synthesized using standard methods. A set of oligonucleotides can be synthesized, for example, by solid phase synthesis, containing sequences that represent all possible combinations of nucleotide triplets provided by the codon set and that will encode the desired group of amino acids. Synthesis of oligonucleotides with selected nucleotide “degeneracy” at certain positions is well known in that art. Such sets of nucleotides having certain codon sets can be synthesized using commercial nucleic acid synthesizers (available from, for example, Applied Biosystems, Foster City, Calif.), or can be obtained commercially (for example, from Life Technologies, Rockville, Md.). Therefore, a set of oligonucleotides synthesized having a particular codon set will typically include a plurality of oligonucleotides with different sequences, the differences established by the codon set within the overall sequence. Oligonucleotides, as used according to the invention, have sequences that allow for hybridization to a variable domain nucleic acid template and also can include restriction enzyme sites for cloning purposes.

In one method, nucleic acid sequences encoding variant amino acids can be created by oligonucleotide-mediated mutagenesis. This technique is well known in the art as described by Zoller et al, 1987, Nucleic Acids Res. 10:6487-6504. Briefly, nucleic acid sequences encoding variant amino acids are created by hybridizing an oligonucleotide set encoding the desired codon sets to a DNA template, where the template is the single-stranded form of the plasmid containing a variable region nucleic acid template sequence. After hybridization, DNA polymerase is used to synthesize an entire second complementary strand of the template that will thus incorporate the oligonucleotide primer, and will contain the codon sets as provided by the oligonucleotide set.

Generally, oligonucleotides of at least 25 nucleotides in length are used. An optimal oligonucleotide will have 12 to 15 nucleotides that are completely complementary to the template on either side of the nucleotide(s) coding for the mutation(s). This ensures that the oligonucleotide will hybridize properly to the single-stranded DNA template molecule. The oligonucleotides are readily synthesized using techniques known in the art such as that described by Crea et al., Proc. Nat'l. Acad. Sci. USA, 75:5765 (1978).

The DNA template is generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153:3 (1987). Thus, the DNA that is to be mutated can be inserted into one of these vectors in order to generate single-stranded template. Production of the single-stranded template is described in sections 4.21-4.41 of Sambrook et al., above.

To alter the native DNA sequence, the oligonucleotide is hybridized to the single stranded template under suitable hybridization conditions. A DNA polymerizing enzyme, usually T7 DNA polymerase or the Klenow fragment of DNA polymerase I, is then added to synthesize the complementary strand of the template using the oligonucleotide as a primer for synthesis. A heteroduplex molecule is thus formed such that one strand of DNA encodes the mutated form of gene 1, and the other strand (the original template) encodes the native, unaltered sequence of gene 1. This heteroduplex molecule is then transformed into a suitable host cell, usually a prokaryote such as E. coli JM101. After growing the cells, they are plated onto agarose plates and screened using the oligonucleotide primer radiolabelled with a 32-Phosphate to identify the bacterial colonies that contain the mutated DNA.

The method described immediately above may be modified such that a homoduplex molecule is created wherein both strands of the plasmid contain the mutation(s). The modifications are as follows: The single stranded oligonucleotide is annealed to the single-stranded template as described above. A mixture of three deoxyribonucleotides, deoxyriboadenosine (dATP), deoxyriboguanosine (dGTP), and deoxyribothymidine (dTT), is combined with a modified thiodeoxyribocytosine called dCTP-(aS) (which can be obtained from Amersham). This mixture is added to the template-oligonucleotide complex. Upon addition of DNA polymerase to this mixture, a strand of DNA identical to the template except for the mutated bases is generated. In addition, this new strand of DNA will contain dCTP-(aS) instead of dCTP, which serves to protect it from restriction endonuclease digestion. After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell.

As indicated previously the sequence of the oligonucleotide set is of sufficient length to hybridize to the template nucleic acid and may also, but does not necessarily, contain restriction sites. The DNA template can be generated by those vectors that are either derived from bacteriophage M13 vectors or vectors that contain a single-stranded phage origin of replication as described by Viera et al. ((1987) Meth. Enzymol., 153:3). Thus, the DNA that is to be mutated must be inserted into one of these vectors in order to generate single-stranded template. Production of the single-stranded template is described in sections 4.21-4.41 of Sambrook et al., supra.

According to another method, a library can be generated by providing upstream and downstream oligonucleotide sets, each set having a plurality of oligonucleotides with different sequences, the different sequences established by the codon sets provided within the sequence of the oligonucleotides. The upstream and downstream oligonucleotide sets, along with a variable domain template nucleic acid sequence, can be used in a polymerase chain reaction to generate a “library” of PCR products. The PCR products can be referred to as “nucleic acid cassettes”, as they can be fused with other related or unrelated nucleic acid sequences, for example, viral coat proteins and dimerization domains, using established molecular biology techniques.

Oligonucleotide sets can be used in a polymerase chain reaction using a variable domain nucleic acid template sequence as the template to create nucleic acid cassettes. The variable domain nucleic acid template sequence can be any portion of the heavy immunoglobulin chains containing the target nucleic acid sequences (ie., nucleic acid sequences encoding amino acids targeted for substitution). The variable region nucleic acid template sequence is a portion of a double stranded DNA molecule having a first nucleic acid strand and complementary second nucleic acid strand. The variable domain nucleic acid template sequence contains at least a portion of a variable domain and has at least one CDR. In some cases, the variable domain nucleic acid template sequence contains more than one CDR. An upstream portion and a downstream portion of the variable domain nucleic acid template sequence can be targeted for hybridization with members of an upstream oligonucleotide set and a downstream oligonucleotide set.

A first oligonucleotide of the upstream primer set can hybridize to the first nucleic acid strand and a second oligonucleotide of the downstream primer set can hybridize to the second nucleic acid strand. The oligonucleotide primers can include one or more codon sets and be designed to hybridize to a portion of the variable region nucleic acid template sequence. Use of these oligonucleotides can introduce two or more codon sets into the PCR product (ie., the nucleic acid cassette) following PCR. The oligonucleotide primer that hybridizes to regions of the nucleic acid sequence encoding the antibody variable domain includes portions that encode CDR residues that are targeted for amino acid substitution.

The upstream and downstream oligonucleotide sets can also be synthesized to include restriction sites within the oligonucleotide sequence. These restriction sites can facilitate the insertion of the nucleic acid cassettes [i.e., PCR reaction products] into an expression vector having additional antibody sequence. In one embodiment, the restriction sites are designed to facilitate the cloning of the nucleic acid cassettes without introducing extraneous nucleic acid sequences or removing original CDR or framework nucleic acid sequences.

Nucleic acid cassettes can be cloned into any suitable vector for expression of a portion or the entire light or heavy chain sequence containing the targeted amino acid substitutions generated via the PCR reaction. According to methods detailed in the invention, the nucleic acid cassette is cloned into a vector allowing production of a portion or the entire light or heavy chain sequence fused to all or a portion of a viral coat protein (i.e., creating a fusion protein) and displayed on the surface of a particle or cell. While several types of vectors are available and may be used to practice this invention, phagemid vectors are the preferred vectors for use herein, as they may be constructed with relative ease, and can be readily amplified. Phagemid vectors generally contain a variety of components including promoters, signal sequences, phenotypic selection genes, origin of replication sites, and other necessary components as are known to those of ordinary skill in the art.

When a particular variant amino acid combination is to be expressed, the nucleic acid cassette contains a sequence that is able to encode all or a portion of the heavy or light chain variable domain, and is able to encode the variant amino acid combinations. For production of antibodies containing these variant amino acids or combinations of variant amino acids, as in a library, the nucleic acid cassettes can be inserted into an expression vector containing additional antibody sequence, for example all or portions of the variable or constant domains of the light and heavy chain variable regions. These additional antibody sequences can also be fused to other nucleic acids sequences, such as sequences that encode viral coat proteins and therefore allow production of a fusion protein.

Methods for conducting alanine scanning mutagenesis are known to those of skill in the art and are described in WO 01/44463 and Morrison and Weiss, Cur. Opin. Chem. Bio., 5:302-307 (2001). Alanine scanning mutagenesis is a site directed mutagenesis method of replacing amino acid residues in a polypeptide with alanine to scan the polypeptide for residues involved in an interaction of interest. Standard site-directed mutagenesis techniques are utilized to systematically substitute individual positions in a protein with an alanine residue. Combinatorial alanine scanning allows multiple alanine substitutions to be assessed in a protein. Amino acid residues are allowed to vary only as the wild type or as an alanine. Utilizing oligonucleotide-mediated mutagenesis or cassette mutagenesis, binomial substitutions of alanine or seven wild type amino acids may be generated. For these seven amino acids, namely aspartic acid, glutamic acid, glycine, proline, serine, threonine, and valine, altering a single nucleotide can result in a codon for alanine. Libraries with alanine substitutions in multiple positions are generated by cassette mutagenesis or degenerate oligonucleotides with mutations in multiple positions. Shotgun scanning utilizes successive rounds of binding selection to enrich residues contributing binding energy to the receptor-ligand interaction.

b. Vectors

One aspect of the invention includes a replicable expression vector comprising a nucleic acid sequence encoding a gene fusion, wherein the gene fusion encodes a fusion protein comprising an antibody variable domain, or an antibody variable domain and a constant domain, fused to all or a portion of a viral coat protein. Also included is a library of diverse replicable expression vectors comprising a plurality of gene fusions encoding a plurality of different fusion proteins including a plurality of the antibody variable domains generated with diverse sequences as described above. The vectors can include a variety of components and are preferably constructed to allow for movement of antibody variable domain between different vectors and/or to provide for display of the fusion proteins in different formats.

Examples of vectors include phage vectors. The phage vector has a phage origin of replication allowing phage replication and phage particle formation. The phage is in certain embodiments a filamentous bacteriophage, such as an M13, f1, fd, Pf3 phage or a derivative thereof, or a lambdoid phage, such as lambda, 21, phi80, phi81, 82, 424, 434, etc., or a derivative thereof.

Examples of viral coat proteins include infectivity protein PIII, major coat protein PVIII, p3, Soc, Hoc, gpD (of bacteriophage lambda), minor bacteriophage coat protein 6 (pVI) (filamentous phage; J. Immunol. Methods, 1999, 231(1-2):39-51), variants of the M13 bacteriophage major coat protein (P8) (Protein Sci 2000 April; 9(4):647-54). The fusion protein can be displayed on the surface of a phage and suitable phage systems include M13KO7 helper phage, M13R408, M13-VCS, and Phi X 174, pJuFo phage system (J. Virol. 2001 August; 75(15):7107-13), hyperphage (Nat. Biotechnol. 2001 January; 19(1):75-8). The preferred helper phage is M13KO7, and the preferred coat protein is the M13 Phage gene III coat protein. The preferred host is E. coli, and protease deficient strains of E. coli. Vectors, such as the fth1 vector (Nucleic Acids Res. 2001 May 15; 29(10):E50-0) can be useful for the expression of the fusion protein.

The expression vector also can have a secretory signal sequence fused to the DNA encoding each subunit of the antibody or fragment thereof. This sequence is typically located immediately 5′ to the gene encoding the fusion protein, and will thus be transcribed at the amino terminus of the fusion protein. However, in certain cases, the signal sequence has been demonstrated to be located at positions other than 5′ to the gene encoding the protein to be secreted. This sequence targets the protein to which it is attached across the inner membrane of the bacterial cell. The DNA encoding the signal sequence may be obtained as a restriction endonuclease fragment from any gene encoding a protein that has a signal sequence. Suitable prokaryotic signal sequences may be obtained from genes encoding, for example, LamB or OmpF (Wong et al., Gene, 68:1931 (1983), MalE, PhoA and other genes. A preferred prokaryotic signal sequence for practicing this invention is the E. coli heat-stable enterotoxin II (STII) signal sequence as described by Chang et al., Gene 55:189 (1987), and malE.

The vector also typically includes a promoter to drive expression of the fusion protein. Promoters most commonly used in prokaryotic vectors include the lac Z promoter system, the alkaline phosphatase pho A promoter, the bacteriophage γ-_{PL} promoter (a temperature sensitive promoter), the tac promoter (a hybrid trp-lac promoter that is regulated by the lac repressor), the tryptophan promoter, and the bacteriophage T7 promoter. For general descriptions of promoters, see section 17 of Sambrook et al. supra. While these are the most commonly used promoters, other suitable microbial promoters may be used as well.

The vector can also include other nucleic acid sequences, for example, sequences encoding gD tags, c-Myc epitopes, poly-histidine tags, fluorescence proteins (e.g., GFP), or beta-galactosidase protein which can be useful for detection or purification of the fusion protein expressed on the surface of the phage or cell. Nucleic acid sequences encoding, for example, a gD tag, also provide for positive or negative selection of cells or virus expressing the fusion protein. In some embodiments, the gD tag is preferably fused to an antibody variable domain which is not fused to the viral coat protein. Nucleic acid sequences encoding, for example, a polyhistidine tag, are useful for identifying fusion proteins including antibody variable domains that bind to a specific antigen using immunohistochemistry. Tags useful for detection of antigen binding can be fused to either an antibody variable domain not fused to a viral coat protein or an antibody variable domain fused to a viral coat protein.

Another useful component of the vectors used to practice this invention are phenotypic selection genes. Typical phenotypic selection genes are those encoding proteins that confer antibiotic resistance upon the host cell. By way of illustration, the ampicillin resistance gene (ampr), and the tetracycline resistance gene (tetr) are readily employed for this purpose.

The vector can also include nucleic acid sequences containing unique restriction sites and suppressible stop codons. The unique restriction sites are useful for moving antibody variable domains between different vectors and expression systems. The suppressible stop codons are useful to control the level of expression of the fusion protein and to facilitate purification of soluble antibody fragments. For example, an amber stop codon can be read as Gln in a supE host to enable phage display, while in a non-supE host it is read as a stop codon to produce soluble antibody fragments without fusion to phage coat proteins. These synthetic sequences can be fused to one or more antibody variable domains in the vector.

It is preferable to use vector systems that allow the nucleic acid encoding an antibody sequence of interest, for example a VH having variant amino acids, to be easily removed from the vector system and placed into another vector system. For example, appropriate restriction sites can be engineered in a vector system to facilitate the removal of the nucleic acid sequence encoding an antibody or antibody variable domain having variant amino acids. The restriction sequences are usually chosen to be unique in the vectors to facilitate efficient excision and ligation into new vectors. Antibodies or antibody variable domains can then be expressed from vectors without extraneous fusion sequences, such as viral coat proteins or other sequence tags.

Between nucleic acid encoding an antibody variable domain (gene 1) and the viral coat protein (gene 2), DNA encoding a termination codon may be inserted, such termination codons including UAG (amber), UAA (ocher) and UGA (opel). (Microbiology, Davis et al., Harper & Row, New York, 1980, pp. 237, 245-47 and 374). The termination codon expressed in a wild type host cell results in the synthesis of the gene 1 protein product without the gene 2 Protein Attached. However, growth in a suppressor host cell results in the synthesis of detectable quantities of fused protein. Such suppressor host cells are well known and described, such as E. coli suppressor strain (Bullock et al., BioTechniques 5:376-379 (1987)). Any acceptable method may be used to place such a termination codon into the mRNA encoding the fusion polypeptide.

The suppressible codon may be inserted between the first gene encoding an antibody variable domain, and a second gene encoding at least a portion of a phage coat protein. Alternatively, the suppressible termination codon may be inserted adjacent to the fusion site by replacing the last amino acid triplet in the antibody variable domain or the first amino acid in the phage coat protein. When the plasmid containing the suppressible codon is grown in a suppressor host cell, it results in the detectable production of a fusion polypeptide containing the polypeptide and the coat protein. When the plasmid is grown in a non-suppressor host cell, the antibody variable domain is synthesized substantially without fusion to the phage coat protein due to termination at the inserted suppressible triplet UAG, UAA, or UGA. In the non-suppressor cell the antibody variable domain is synthesized and secreted from the host cell due to the absence of the fused phage coat protein which otherwise anchored it to the host membrane.

In some embodiments, the VH FR and/or CDR being diversified (randomized) may have a stop codon engineered in the template sequence (referred to herein as a “stop template”). This feature provides for detection and selection of successfully diversified sequences based on successful repair of the stop codon(s) in the template sequence due to incorporation of the oligonucleotide(s) comprising the sequence(s) for the variant amino acids of interest. This feature is further illustrated in the Examples herein.

The light and/or heavy antibody variable domains can also be fused to an additional peptide sequence, the additional peptide sequence allowing the interaction of one or more fusion polypeptides on the surface of the viral particle or cell. These peptide sequences are herein referred to as “dimerization sequences”, “dimerization peptides” or “dimerization domains”. Suitable dimerization domains include those of proteins having amphipathic alpha helices in which hydrophobic residues are regularly spaced and allow the formation of a dimer by interaction of the hydrophobic residues of each protein; such proteins and portions of proteins include, for example, leucine zipper regions. The dimerization regions can be located between the antibody variable domain and the viral coat protein.

In some cases the vector encodes a single antibody-phage polypeptide in a single chain form containing, for example, the heavy chain variable region fused to a coat protein. In these cases the vector is considered to be “monocistronic”, expressing one transcript under the control of a certain promoter. A vector may utilize an alkaline phosphatase (AP) or Tac promoter to drive expression of a monocistronic sequence encoding VL and VH domains, with a linker peptide between the VL and VH domains. This cistronic sequence is connected at the 5′ end to an E. coli malE or heat-stable enterotoxin II (STII) signal sequence and at its 3′ end to all or a portion of a viral coat protein. In some embodiments, the vector may further comprise a sequence encoding a dimerization domain (such as a leucine zipper) at its 3′ end, between the second variable domain sequence and the viral coat protein sequence. Fusion polypeptides comprising the dimerization domain are capable of dimerizing to form a complex of two scFv polypeptides (referred to herein as “(ScFv)2-pIII)”).

In other cases, e.g., the variable regions of the heavy and light chains can be expressed as separate polypeptides, the vector thus being “bicistronic”, allowing the expression of separate transcripts. In these vectors, a suitable promoter, such as the Ptac or PhoA promoter, can be used to drive expression of a bicistronic message. A first cistron, encoding, for example, a light chain variable domain, is connected at the 5′ end to a E. coli malE or heat-stable enterotoxin II (STII) signal sequence and at the 3′ end to a nucleic acid sequence encoding a gD tag. A second cistron, encoding, for example, a heavy chain variable domain, is connected at its 5′ end to an E. coli malE or heat-stable enterotoxin II (STII) signal sequence and at the 3′ end to all or a portion of a viral coat protein.

c. Introduction of Vectors into Host Cells

Vectors constructed as described in accordance with the invention are introduced into a host cell for amplification and/or expression. Vectors can be introduced into host cells using standard transformation methods including electroporation, calcium phosphate precipitation and the like. If the vector is an infectious particle such as a virus, the vector itself provides for entry into the host cell. Transfection of host cells containing a replicable expression vector which encodes the gene fusion and production of phage particles according to standard procedures provides phage particles in which the fusion protein is displayed on the surface of the phage particle.

Replicable expression vectors are introduced into host cells using a variety of methods. In one embodiment, vectors can be introduced into cells using electroporation as described in WO/00106717. Cells are grown in culture in standard culture broth, optionally for about 6-48 hours (or to OD₆₀₀=0.6-0.8) at about 37° C., and then the broth is centrifuged and the supernatant removed (e.g. decanted). Initial purification is, e.g., by resuspending the cell pellet in a buffer solution (e.g. 1.0 mM HEPES pH 7.4) followed by recentrifugation and removal of supernatant. The resulting cell pellet is resuspended in dilute glycerol (e.g. 5-20% v/v) and again recentrifuged to form a cell pellet and the supernatant removed. The final cell concentration is obtained by resuspending the cell pellet in water or dilute glycerol to the desired concentration.

A particularly preferred recipient cell is the electroporation competent E. coli strain of the present invention, which is E. coli strain SS320 (Sidhu et al., Methods Enzymol. (2000), 328:333-363). Strain SS320 was prepared by mating MC1061 cells with XL1-BLUE cells under conditions sufficient to transfer the fertility episome (F′ plasmid) or XL1-BLUE into the MC1061 cells. Strain SS320 has been deposited with the American Type Culture Collection (ATCC), 10801 University Boulevard, Manassas, Va. USA, on Jun. 18, 1998 and assigned Deposit Accession No. 98795. Any F′ episome which enables phage replication in the strain may be used in the invention. Suitable episomes are available from strains deposited with ATCC or are commercially available (CJ236, CSH18, DHF′, JM101, JM103, JM105, JM107, JM109, JM110), KS1000, XL1-BLUE, 71-18 and others).

The use of higher DNA concentrations during electroporation (about 10×) increases the transformation efficiency and increases the amount of DNA transformed into the host cells. The use of high cell concentrations also increases the efficiency (about 10×). The larger amount of transferred DNA produces larger libraries having greater diversity and representing a greater number of unique members of a combinatorial library. Transformed cells are generally selected by growth on antibiotic containing medium.

d. Display of Fusion Polypeptides

Fusion polypeptides comprising an antibody variable domain can be displayed on the surface of a cell or virus in a variety of formats. These formats include, but are not limited to, single chain Fv fragment (scFv), F(ab) fragment, variable domain of a monobody and multivalent forms of these fragments. The multivalent forms can be a dimer of ScFv, Fab, or F(ab)′, herein referred to as (ScFv)₂, F(ab)₂ and F(ab)′₂, respectively. The multivalent forms of display are preferred in part because they have more than one antigen binding site which generally results in the identification of lower affinity clones and also allows for more efficient sorting of rare clones during the selection process.

Methods for displaying fusion polypeptides comprising antibody fragments, on the surface of bacteriophage, are well known in the art, for example as described in patent publication number WO 92/01047 and herein. Other patent publications WO 92/20791; WO 93/06213; WO 93/11236 and WO 93/19172, describe related methods and are all herein incorporated by reference. Other publications have shown the identification of antibodies with artificially rearranged V gene repertoires against a variety of antigens displayed on the surface of phage (for example, Hoogenboom & Winter, 1992, J. Mol. Biol., 227: 381-388; and as disclosed in WO 93/06213 and WO 93/11236).

When a vector is constructed for display in a scFv format, it includes nucleic acid sequences encoding an antibody variable light chain domain and an antibody variable heavy chain variable domain. Typically, the nucleic acid sequence encoding an antibody variable heavy chain domain is fused to a viral coat protein. One or both of the antibody variable domains can have variant amino acids in at least one CDR or FR. The nucleic acid sequence encoding the antibody variable light chain is connected to the antibody variable heavy chain domain by a nucleic acid sequence encoding a peptide linker. The peptide linker typically contains about 5 to 15 amino acids. Optionally, other sequences encoding, for example, tags useful for purification or detection can be fused at the 3′ end of either the nucleic acid sequence encoding the antibody variable light chain or antibody variable heavy chain domain or both.

When a vector is constructed for F(ab) display, it includes nucleic acid sequences encoding antibody variable domains and antibody constant domains. A nucleic acid encoding a variable light chain domain is fused to a nucleic acid sequence encoding a light chain constant domain. A nucleic acid sequence encoding an antibody heavy chain variable domain is fused to a nucleic acid sequence encoding a heavy chain constant CH1 domain. Typically, the nucleic acid sequence encoding the heavy chain variable and constant domains are fused to a nucleic acid sequence encoding all or part of a viral coat protein. One or both of the antibody variable light or heavy chain domains can have variant amino acids in at least one CDR and/or FR. The heavy chain variable and constant domains are in one embodiment expressed as a fusion with at least a portion of a viral coat and the light chain variable and constant domains are expressed separately from the heavy chain viral coat fusion protein. The heavy and light chains associate with one another, which may be by covalent or non-covalent bonds. Optionally, other sequences encoding, for example, polypeptide tags useful for purification or detection, can be fused at the 3′ end of either the nucleic acid sequence encoding the antibody light chain constant domain or antibody heavy chain constant domain or both.

In one embodiment, a bivalent moiety, for example, a F(ab)₂ dimer or F(ab)′₂ dimer, is used for displaying antibody fragments with the variant amino acid substitutions on the surface of a particle. It has been found that F(ab)′₂ dimers have the same affinity as F(ab) dimers in a solution phase antigen binding assay but the off rate for F(ab)′₂ are reduced because of a higher avidity in an assay with immobilized antigen. Therefore the bivalent format (for example, F(ab)′₂) is a particularly useful format since it can allow the identification of lower affinity clones and also allows more efficient sorting of rare clones during the selection process.

6. Fusion Polypeptides

Fusion polypeptide constructs can be prepared for generating fusion polypeptides that bind with significant affinity to potential ligands. In particular, fusion polypeptides comprising an isolated VH with one or more amino acid alterations that increase the stability of the polypeptide and a heterologous polypeptide sequence (e.g., that of at least a portion of a viral polypeptide) are generated, individually and as a plurality of unique individual polypeptides that are candidate binders to targets of interest. Compositions (such as libraries) comprising such polypeptides find use in a variety of applications, in particular as large and diverse pools of candidate immunoglobulin polypeptides (in particular, antibodies and antibody fragments) that bind to targets of interest.

In some embodiments, a fusion protein comprises an isolated VH, or a VH and a constant domain, fused to all or a portion of a viral coat protein. Examples of viral coat proteins include infectivity protein PIII, major coat protein PVIII, p3, Soc, Hoc, gpD (of bacteriophage lambda), minor bacteriophage coat protein 6 (pVI) (filamentous phage; J. Immunol. Methods. 1999 Dec. 10; 231(1-2):39-51), variants of the M13 bacteriophage major coat protein (P8) (Protein Sci. 2000 April; 9(4):647-54). The fusion protein can be displayed on the surface of a phage and suitable phage systems include M13KO7 helper phage, M13R408, M13-VCS, and Phi X 174, pJuFo phage system (J. Virol. 2001 August; 75(15):7107-13.v), hyperphage (Nat. Biotechnol. 2001 January; 19(1):75-8). In one embodiment, the helper phage is M13KO7, and the coat protein is the M13 Phage gene III coat protein.

Tags useful for detection of antigen binding can also be fused to either an antibody variable domain not fused to a viral coat protein or an antibody variable domain fused to a viral coat protein. Additional peptides that can be fused to antibody variable domains include gD tags, c-Myc epitopes, poly-histidine tags, fluorescence proteins (e.g., GFP), or β-galactosidase protein which can be useful for detection or purification of the fusion protein expressed on the surface of the phage or cell.

In certain embodiments, the stability and/or half-life of a VH domain of the invention is modulated by fusing or otherwise associating one or more additional molecules to the VH domain. Isolated VH domains are relatively small molecules, and the addition of one or more fusion partners (either active partners, such as, but not limited to, one or more additional VH or VL domains, an enzyme, or another binding partner, or nonfunctional partners, such as, but not limited to, albumin) increases the size of the protein and may decrease its rate of clearance in vivo. Another approach known in the art is to increase the size of a protein by increasing the amount of posttranslational modification that the protein undergoes. As nonlimiting examples, additional glycosylation sites can be added within the protein, or the protein can be PEGylated, as is known in the art. Another approach to increasing circulating half-life of VH domains is to associate them with another VH or VL domain that binds serum albumin (see, e.g., EP1517921B).

These VH domain constructs may also comprise a dimerizable sequence that when present as a dimerization domain in a fusion polypeptide provides for increased tendency for heavy chains to dimerize to form dimers of Fab or Fab′ antibody fragments/portions. These dimerization sequences may be in addition to any heavy chain hinge sequence that may be present in the fusion polypeptide. Dimerization domains in fusion phage polypeptides bring two sets of fusion polypeptides (LC/HC-phage protein/fragment (such as pIII)) together, thus allowing formation of suitable linkages (such as interheavy chain disulfide bridges) between the two sets of fusion polypeptide. Vector constructs containing such dimerization sequences can be used to achieve divalent display of antibody variable domains, for example the diversified fusion proteins described herein, on phage. In one embodiment, the intrinsic affinity of each monomeric antibody fragment (fusion polypeptide) is not significantly altered by fusion to the dimerization sequence. In another embodiment, dimerization results in divalent phage display which provides increased avidity of phage binding, with significant decrease in off-rate, which can be determined by methods known in the art and as described herein. Dimerization sequence-containing vectors of the invention may or may not also include an amber stop codon 5′ of the dimerization sequence. Dimerization sequences are known in the art, and include, for example, the GCN4 zipper sequence (GRMKQLEDKVEELLSKNYHLENEVARLKKLVGERG) (SEQ ID NO: 250).

It is contemplated that the isolated VH domains described herein or obtained using the methodologies described herein may be employed as isolated VH domains, or may be combined with one or more other VH domains to form an antibody- or antibody fragment-like structure. Methods of incorporating one or more VH domains into an antibody-like or antibody fragment-like structure are well known in the art, and such antibody-like or antibody-fragment-like structures may contain one or more framework regions, constant regions, or other portions of one or more native or synthetic antibodies sufficient to maintain the one or more VH domains in a spatial orientation in which they are capable of binding to a target. In certain embodiments, a molecule comprising two or more isolated VH domains is specific for a single target. In certain embodiments, a molecule comprising two or more isolated VH domains is specific for more than one target. In certain embodiments, a molecule comprising two or more isolated VH domains is bispecific.

It is further contemplated that the isolated VH domains described herein may be associated with another molecule while retaining their binding properties. In a nonlimiting example, one or more isolated VH domains of the invention may be associated with an antibody, an scFv, a heavy chain of an antibody, a light chain of an antibody, a Fab fragment of an antibody, or an F(ab)₂ fragment of an antibody. Such association may be covalent (i.e., by direct fusion or by indirect fusion via one or more linking molecules) or noncovalent (i.e., by disulfide bond, charge-charge interaction, biotin-streptavidin linkage, or other noncovalent association known in the art).

7. Antibodies

The libraries described herein may be used to isolate antibodies, antibody fragments, monobodies, or antibody variable domains specific for an antigen of choice. Monobodies are antigen binding molecules that lack light chains. Although their antigen combining site is found only in a heavy chain variable domain, the affinities for antigens have been found to be similar to those of classical antibodies (Ferrat et al., Biochem J., 366:415 (2002)). Because monobodies bind their targets with high affinity and specificity, monobodies may used as modules in the design of traditional antibodies. A traditional antibody may be constructed by converting a high affinity heavy chain antibody or monobody to a Fab or IgG and pairing the converted heavy chain antibody or monobody with an appropriate light chain. The monobodies may also be utilized to form novel antigen binding molecules or mini-antibodies without the need for any light chain. These novel mini-antibodies or antigen binding molecules are similar to other single chain type antibodies, but the antigen binding domain is a heavy chain variable domain.

Antibody variable domains specific for a target antigen can be combined with each other or with constant regions to form an antigen binding antibody fragment or full length antibody. These antibodies can be used in purification, diagnostic and in therapeutic applications. It will be understood that in certain embodiments described herein, variant isolated heavy chain antibody variable domains have modifications that enhance the stability of the isolated heavy chain antibody variable domain in the absence of a light chain, and which may concomitantly decrease the ability of the isolated heavy chain antibody variable domain to associate with a light chain variable domain. Thus, in certain embodiments where a VH domain of the invention is combined into a single molecule with a VL domain, recombinant methods may be used to overcome such a decrease in binding affinity between the VH domain of the invention and a VL domain. Such methods are well known to those of ordinary skill in the art and include, e.g., genetically or chemically fusing the VH domain to the VL domain.

8. Uses and Methods

The invention provides novel methods for diversifying heavy chain antibody variable domain sequences such that their stability is enhanced, and also provides libraries comprising a multiplicity, generally a great multiplicity, of diversified heavy chain antibody variable domain sequences with enhanced folding stability. Such libraries are useful for, for example, screening for synthetic antibody or antigen binding polypeptides with desirable activities such as binding affinities and avidities. Such libraries provide a tremendously useful resource for identifying immunoglobulin polypeptide sequences that are capable of interacting with any of a wide variety of target molecules. For example, libraries comprising diversified immunoglobulin polypeptides of the invention expressed as phage displays are particularly useful for, and provide a high throughput for, efficient and automatable systems of screening for antigen binding molecules of interest. In some embodiments, the diversified antibody variable domains are provided in a monobody that binds to antigen in the absence of light chains. The population of variant VH, optionally in combination with one or more variant CDRs, can then be utilized in libraries to identify novel antigen binding molecules with desired stability.

Also provided are methods for designing VH regions that can be used to generate a plurality of stable VH regions. The invention provides methods for generating and isolating novel antibodies or antigen binding fragments or antibody variable domains with high folding stability that preferably have a high affinity for a selected antigen. A plurality of different antibodies or antibody variable domains are prepared by mutating (diversifying) one or more selected amino acid positions in a source heavy chain variable domain to generate a diverse library of antigen binding variable domains with variant amino acids at those positions. The diversity in the isolated heavy chain variable domains is designed so that highly diverse libraries are obtained with increased folding stability. In one aspect, the amino acid positions selected for variation are one or more amino acid positions that interact with the VL, for example as determined by analyzing the structure of a source antibody and/or natural immunoglobulin polypeptides. In another aspect, the amino acid positions selected for variation include one or more amino acid positions that interact with the VL and further include one or more amino acid positions in one or more CDRs. In another aspect, the amino acid positions are those positions in a VH region that are structural, and for which diversity is limited while the remaining positions can be randomized to generate a library that is highly diverse and well folded.

Variable domain fusion proteins expressing the variant amino acids can be expressed on the surface of a phage or a cell and then screened for the ability of members of the group of fusion proteins to specifically bind a target molecule, such as a target protein, which is typically an antigen of interest or is a molecule that binds to folded polypeptide and does not bind to unfolded polypeptide or both. Target proteins may include protein L or Protein A which specifically binds to antibody or antibody fragments and can be used to enrich for library members that display correctly folded antibody fragments (fusion polypeptides). In another embodiment, a target molecule is a molecule that specifically binds to folded polypeptide and does not bind to unfolded polypeptide and does not bind at an antigen binding site. For example, the Protein A binding site of Vh3 antibody variable domains are found on the opposite B sheet from the antigen binding site. Another example of a target molecule includes an antibody or antigen binding fragment or polypeptide that does not bind to the antigen binding site and binds to folded polypeptide and does not bind to unfolded polypeptide, such as an antibody to the Protein A binding site. Target proteins can also include specific antigens, such as receptors, and may be isolated from natural sources or prepared by recombinant methods by procedures known in the art.

Screening for the ability of a fusion polypeptide to bind a target molecule can also be performed in solution phase. For example, a target molecule can be attached with a detectable moiety, such as biotin. Phage that binds to the target molecule in solution can be separated from unbound phage by a molecule that binds to the detectable moiety, such as streptavidin-coated beads where biotin is the detectable moiety. Affinity of binders (fusion polypeptide that binds to target) can be determined based on concentration of the target molecule used, using formulas and based on criteria known in the art.

Target antigens can include a number of molecules of therapeutic interest. Included among cytokines and growth factors are growth hormone, bovine growth hormone, insulin like growth factors, human growth hormone including n-methionyl human growth hormone, parathyroid hormone, thyroxine, insulin, proinsulin, amylin, relaxin, prorelaxin, glycoprotein hormones such as follicle stimulating hormone(FSH), leutinizing hormone (LH), hematopoietic growth factor, fibroblast growth factor, prolactin, placental lactogen, tumor necrosis factors, mullerian inhibiting substance, mouse gonadotropin-associated polypeptide, inhibin, activin, vascular endothelial growth factors, integrin, nerve growth factors such as NGF-beta, insulin-like growth factor-I and II, erythropoietin, osteoinductive factors, interferons, colony stimulating factors, interleukins, bone morphogenetic proteins, LIF,SCF,FLT-3 ligand and kit-ligand.

The purified target protein may be attached to a suitable matrix such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described in Methods in Enzymology, 44 (1976), or by other means known in the art.

After attachment of the target protein to the matrix, the immobilized target is contacted with the library expressing the fusion polypeptides under conditions suitable for binding of at least a portion of the phage particles with the immobilized target. Normally, the conditions, including pH, ionic strength, temperature and the like will mimic physiological conditions. Bound particles (“binders”) to the immobilized target are separated from those particles that do not bind to the target by washing. Wash conditions can be adjusted to result in removal of all but the higher affinity binders. Binders may be dissociated from the immobilized target by a variety of methods. These methods include competitive dissociation using the wild-type ligand, altering pH and/or ionic strength, and methods known in the art. Selection of binders typically involves elution from an affinity matrix with a ligand. Elution with increasing concentrations of ligand should elute displayed binding molecules of increasing affinity.

The binders can be isolated and then reamplified or expressed in a host cell and subjected to another round of selection for binding of target molecules. Any number of rounds of selection or sorting can be utilized. One of the selection or sorting procedures can involve isolating binders that bind to protein L or an antibody to a polypeptide tag such as antibody to the gD protein or polyhistidine tag. Another selection or sorting procedure can involve multiple rounds of sorting for stability, such as binding to a target molecule that specifically binds to folded polypeptide and does not bind to unfolded polypeptide followed by selecting or sorting the stable binders for binding to an antigen (such as VEGF).

In some cases, suitable host cells are infected with the binders and helper phage, and the host cells are cultured under conditions suitable for amplification of the phagemid particles. The phagemid particles are then collected and the selection process is repeated one or more times until binders having the desired affinity for the target molecule are selected. In certain embodiments, at least two rounds of selection are conducted.

After binders are identified by binding to the target antigen, the nucleic acid can be extracted. Extracted DNA can then be used directly to transform E. coli host cells or alternatively, the encoding sequences can be amplified, for example using PCR with suitable primers, and then inserted into a vector for expression.

A preferred strategy to isolate high affinity binders is to bind a population of phage to an affinity matrix which contains a low amount of ligand. Phage displaying high affinity polypeptide is preferentially bound and low affinity polypeptide is washed away. The high affinity polypeptide is then recovered by elution with the ligand or by other procedures which elute the phage from the affinity matrix.

In certain embodiments, the process of screening is carried out by automated systems to allow for high-throughput screening of library candidates.

In some cases the novel VH sequences described herein can be combined with other sequences generated by introducing variant amino acids via codon sets into CDRs in the heavy and/or light chains, for example through a 2-step process. An example of a 2-step process comprises first determining binders (generally lower affinity binders) within one or more libraries generated by randomizing VH FRs, and optionally one or more CDRs, wherein the VH FR is randomized and each library is different or, where the same domain is randomized, it is randomized to generate different sequences. VH framework region and/or CDR diversity from binders from a heavy chain library can then be combined with CDR diversity from binders from a light chain library (e.g. by ligating different CDR sequences together). The pool can then be further sorted against target to identify binders possessing increased affinity. Novel antibody sequences can be identified that display higher binding affinity to one or more target antigens.

In some embodiments, libraries comprising polypeptides of the invention are subjected to a plurality of sorting rounds, wherein each sorting round comprises contacting the binders obtained from the previous round with a target molecule distinct from the target molecule(s) of the previous round(s). Preferably, but not necessarily, the target molecules are homologous in sequence, for example members of a family of related but distinct polypeptides, including, but not limited to, cytokines (for example, alpha interferon subtypes).

Another aspect of the invention involves a method of designing an isolated VH region that is well folded and stable for phage display. The method involves generating a library comprising polypeptides with variant VH regions, selecting the members of the library that bind to a target molecule that binds to folded polypeptide and does not bind to unfolded polypeptide, analyzing the members of the library to identify structural amino acid positions in the isolated VH region, identifying at least one amino acid that can be substituted at the structural amino acid position, wherein the amino acid identified is one that occurs significantly more frequently than random (one standard deviation or greater than the frequency of any amino acid at that position) in polypeptides selected for stability, and designing an isolated VH region that has at least one or the identified amino acids in the structural amino acid position.

It is contemplated that the sequence diversity of libraries created by introduction of variant amino acids in VH by any of the embodiments described herein can be increased by combining these VH variations with variations in other regions of the antibody, specifically in CDRs of either the light and/or heavy chain variable sequences. It is contemplated that the nucleic acid sequences that encode members of this set can be further diversified by introduction of other variant amino acids in the CDRs of either the light or heavy chain sequences, via codon sets. Thus, for example, in one embodiment, an isolated VH sequence described herein that has a variation at one or more FR amino acid positions and that binds a target antigen can be combined with diversified CDRH1, CDRH2, or CDRH3 sequences, or any combination of diversified CDRs.

Another aspect of the invention involves a method of generating a population of variant VH polypeptides comprising identifying VH amino acid positions involved in interfacing with VL; and replacing the amino acid in at least one such amino acid position with at least one alternate amino acid to generate a population of polypeptides that have different amino acid sequences in VH. In one such aspect, an amino acid position in the VH polypeptide is replaced with the most commonly occurring amino acids at that position in a population of polypeptides with randomized VH.

The method may further comprise generating a plurality of such isolated VH that further have a variant CDR-H1. The method may further comprise generating a plurality of such isolated VH with a variant CDR2. The method may further comprise generating a plurality of such isolated VH with a variant CDR3.

Another aspect of the invention is a method of generating a scaffold heavy chain antibody variable domain with increased folding stability relative to a wild-type heavy chain antibody variable domain. The method involves generating a library of antibody variable domains randomized at each amino acid position in the VH. The library is sorted against a target molecule that binds to folded polypeptide and does not bind to unfolded polypeptide, e.g., in one embodiment, Protein A. The library is further sorted using one or more methodologies to assess folding stability. Multiple rounds of amplification and selection may take place. In certain embodiments, at least three rounds of amplification and selection are conducted. At the fourth or fifth rounds, the sequence of each of the four most dominant clones is identified. The identity of the structural amino acid positions in any particular clone may be confirmed using, for example, combinatorial alanine scanning mutagenesis. A VH scaffold with increased folding stability relative to a wild-type VH polypeptide is then prepared by limiting the diversity at the identified structural amino acid positions and modifying one or more nonstructural amino acid positions identified in the screening and selection process to enhance the folding stability of the isolated VH domain.

A protein of the present invention (e.g., a VH domain, or an antibody, antibody fragment, or fusion protein comprising such VH domain) may also be used in, for example, in vitro, ex vivo and in vivo therapeutic methods. A protein of the invention can be used as an antagonist to partially or fully block the specific antigen activity in vitro, ex vivo and/or in vivo. Moreover, at least some of the proteins of the invention can neutralize antigen activity from other species. Accordingly, the proteins of the invention can be used to inhibit a specific antigen activity, e.g., in a cell culture containing the antigen, in human subjects or in other mammalian subjects having the antigen with which a protein of the invention cross-reacts (e.g. chimpanzee, baboon, marmoset, cynomolgus and rhesus, pig or mouse). In one embodiment, the protein of the invention can be used for inhibiting antigen activities by contacting a protein of the invention with the antigen such that antigen activity is inhibited. In certain embodiments, the antigen is a human protein molecule.

In one embodiment, a protein of the invention (e.g., a VH domain of the invention, or an antibody, antibody fragment, or fusion protein comprising such VH domain), can be used in a method for inhibiting an antigen in a subject suffering from a disorder in which the antigen activity is detrimental, comprising administering to the subject a protein of the invention such that the antigen activity in the subject is inhibited. In certain embodiments, the antigen is a human protein molecule and the subject is a human subject. Alternatively, the subject can be a mammal expressing the antigen with which a protein of the invention binds. Still further the subject can be a mammal into which the antigen has been introduced (e.g., by administration of the antigen or by expression of an antigen transgene). A protein of the invention can be administered to a human subject for therapeutic purposes. Moreover, a protein of the invention can be administered to a non-human mammal expressing an antigen with which the protein of the invention cross-reacts (e.g., a primate, pig or mouse) for veterinary purposes or as an animal model of human disease. Regarding the latter, such animal models may be useful for evaluating the therapeutic efficacy of proteins of the invention (e.g., testing of dosages and time courses of administration).

In one aspect, a protein of the invention (e.g., a VH domain of the invention or an antibody, antibody fragment, or fusion protein comprising such VH domain) with blocking activity against one or more target antigens is specific for a ligand antigen, and inhibits the antigen activity by blocking or interfering with the ligand-receptor interaction involving the ligand antigen, thereby inhibiting the corresponding signal pathway and other molecular or cellular events. In another aspect, a protein of the invention may be specific for one or more receptors, and interfere with receptor activation while not necessarily preventing ligand binding. In certain embodiments, proteins of the invention may exclusively bind to ligand-receptor complexes. A protein of the invention can also act as an agonist of a particular antigen receptor, thereby potentiating, enhancing or activating either all or partial activities of the ligand-mediated receptor activation.

In certain embodiments, a fusion protein comprising a VH domain of the invention conjugated with a cytotoxic agent is administered to the patient. In one aspect, such a fusion protein and/or antigen to which it is bound is/are internalized by a cell, resulting in increased therapeutic efficacy of the fusion protein in killing the target cell to which it binds. In another aspect, the cytotoxic agent targets or interferes with nucleic acid in the target cell. Examples of such cytotoxic agents include many chemotherapeutic agents well known in the art (including, but not limited to, a maytansinoid or a calicheamicin), a radioactive isotope, or a ribonuclease or a DNA endonuclease.

Antibodies of the invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Such combined therapies noted above include combined administration (where the two or more agents are included in the same or separate formulations), and separate administration, in which case, administration of the antibody of the invention can occur prior to, and/or following, administration of the adjunct therapy or therapies.

The protein of the invention (e.g., a VH domain of the invention, or an antibody, antibody fragment, or fusion protein comprising such VH domain) (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the protein of the invention may be suitably administered by pulse infusion, particularly with declining doses of the protein. Dosing can be by any suitable route, for example by injections, such as intravenous or subcutaneous injections, depending in part on whether the administration is brief or chronic.

A composition of a protein of the invention (e.g., a VH domain of the invention, or an antibody, antibody fragment, or fusion protein comprising such VH domain) will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The protein of the invention need not be, but can be optionally formulated with one or more agents currently used to prevent or treat the disorder in question. The effective amount of such other agents depends on the amount of protein of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an protein of the invention (e.g., a VH domain of the invention or an antibody or an antibody, antibody fragment, or fusion protein comprising such VH domain) (when used alone or in combination with other agents such as chemotherapeutic agents) will depend on the type of disease to be treated, the type of protein, the severity and course of the disease, whether the protein is administered for preventive or therapeutic purposes, previous therapy, the patient's clinical history and response to the protein, and the discretion of the attending physician. The protein of the invention is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of a protein of the invention would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of a protein of the invention). An initial higher loading dose, followed by one or more lower doses may be administered. An exemplary dosing regimen comprises administering an initial loading dose of about 4 mg/kg, followed by a weekly maintenance dose of about 2 mg/kg of a protein of the invention. However, other dosage regimens may be useful. The progress of this therapy is easily monitored by conventional techniques and assays.

In another embodiment, an article of manufacture containing materials useful for the treatment, prevention and/or diagnosis of one or more disorders is provided, comprising a container and a label or package insert on or associated with the container. Suitable containers include, for example, bottles, vials, syringes, etc. The containers may be formed from a variety of materials such as glass or plastic. The container holds a composition which is by itself or when combined with another composition effective for treating, preventing and/or diagnosing the condition and may have a sterile access port (for example the container may be an intravenous solution bag or a vial having a stopper pierceable by a hypodermic injection needle). At least one active agent in the composition is a protein of the invention (e.g., a VH domain, or an antibody, antibody fragment, or fusion protein comprising such VH domain). The label or package insert indicates that the composition is used for treating the condition of choice, such as cancer. Moreover, the article of manufacture may comprise (a) a first container with a composition contained therein, wherein the composition comprises a protein of the invention; and (b) a second container with a composition contained therein, wherein the composition comprises a further cytotoxic agent. The article of manufacture in this embodiment of the invention may further comprise a package insert indicating that the first and second protein compositions can be used to treat a particular condition, for example cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

All publications (including patents and patent applications) cited herein are hereby incorporated in their entirety by reference.

Having generally described the invention, the same will be more readily understood by reference to the following examples, which are provided by way of illustration and are not intended as limiting.

### EXAMPLES

### Example 1

### Construction, Sorting, and Analysis of Phage-displayed VH Library 1

A. Preparation of Parental Phagemid Construct

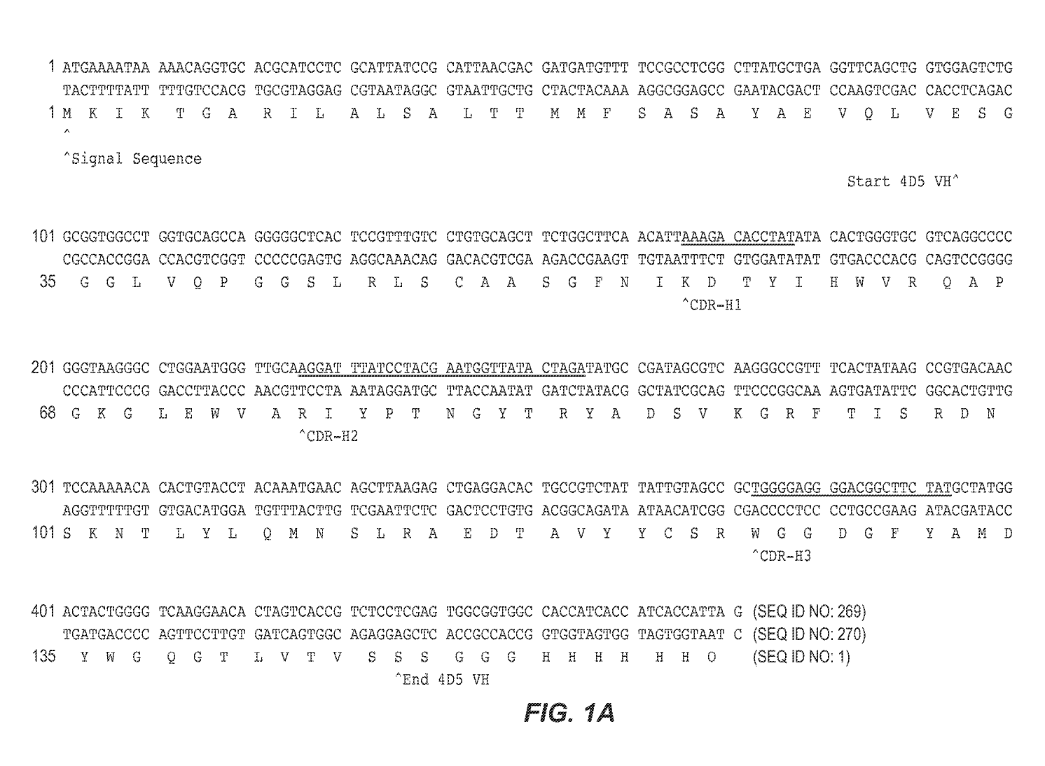

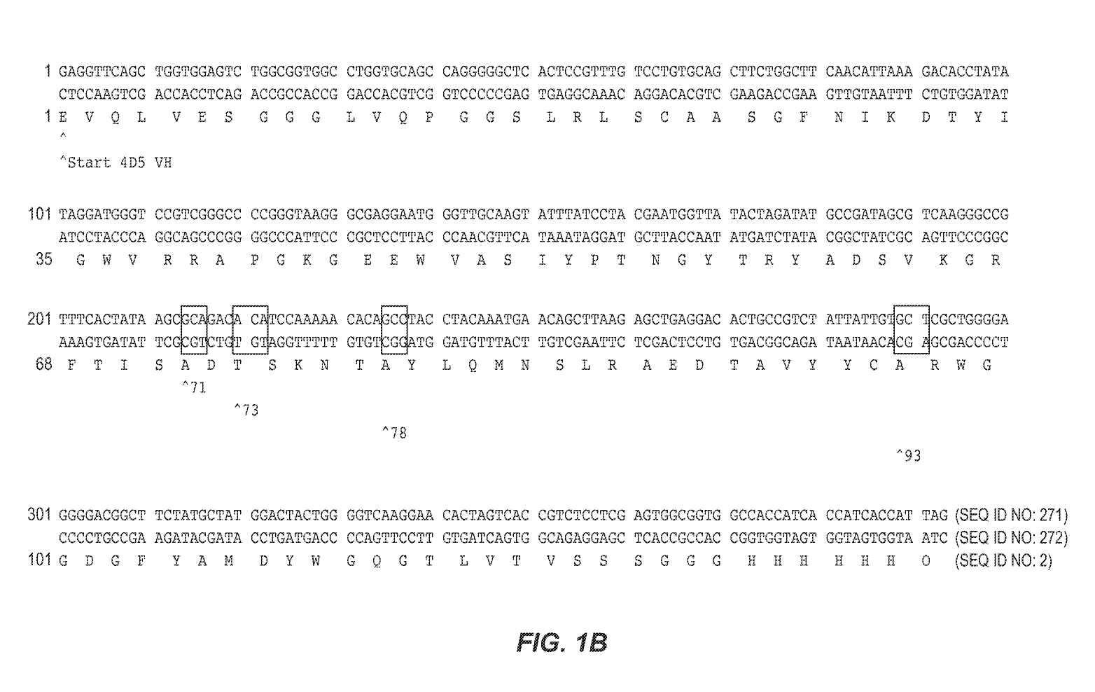

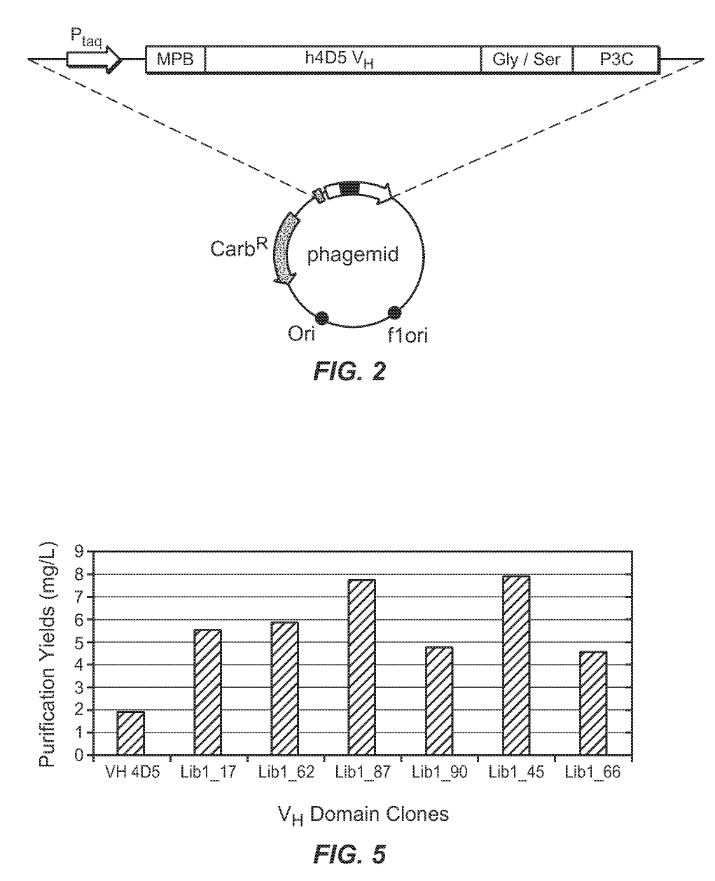

The VH domain of human antibody 4D5 (Herceptin™) was selected as the parent scaffold for library construction. The amino acid sequence of the 4D5 VH domain used for the following experiments appears in FIG. 1A (SEQ ID NO: 3). The 4D5 VH domain is a member of the VH3 family and binds to Protein A. A phagemid was constructed by insertion of a nucleic acid sequence encoding the open reading frame of the 4D5 VH domain into a phagemid construct using standard molecular biology techniques. The resulting construct, pPAB43431-7, encoded a 4D5 VH domain fusion construct under the control of the IPTG-inducible Ptaq promoter. From the N-terminus to the C-terminus, the 4D5 VH domain fusion protein comprised: a maltose-binding protein signal peptide, the 4D5 VH domain, a Gly/Ser-rich linker peptide, and P3C, as shown in FIG. 2.

B. Construction of Library 1

The relative importance of the length of CDR-H3 and the presence of the main camelid residues (amino acid positions 37, 45, and 47) as well as previously identified residue 35 were investigated as potential contributors to isolated VH folding and stability. A human VH domain phage-displayed library was constructed using the pPAB43431-7 construct using a previously described methodology (Sidhu et al., Meth. Enzymol. 328: 333-363 (2000)). Within the construct, VH amino acid positions 35, 37, 45, and 47 were replaced by degenerate codons, and 7 to 17 degenerate codons were also permitted between amino acid positions 92 and 103 (within CDR-H3).

Prior to library construction, phagemid pPAB43431-7 was modified using the Kunkel mutagenesis method by introducing TAA stop codons at locations where the phagemid was to be mutated. For Library 1, two stop-codon-encoding oligonucleotides were used: A1: ACT GCC GTC TAT TAT TGT TAA TAA TAA TGG GGT CAA GGA ACA CTA (SEQ ID NO: 247) and A3: GAC ACC TAT ATA CAC TGG TAA CGT CAG GCC CCG GGT AAG GGC TAA GAA TGG GTT GCA AGG ATT (SEQ ID NO: 248). The resulting “Stop Template” version of pPAB43431-7 was used as the template in a second round of Kunkel mutagenesis with degenerate oligonucleotides designed to simultaneously (a) repair the stop codons and (b) introduce the desired mutations. The oligonucleotides used for the mutagenesis reaction were:

(SEQ ID NO: 7)

Oligo 1-1.
ATT AAA GAC ACC TAT ATA NNS TGG NNS CGT CAG GCC
CCG GGT AAG GGC NNS GAA NNS GTT GCA AGG ATT TAT
CTT

(SEQ ID NO: 8)

Oligo 1-2.
ACT GCC GTC TAT TAT TGT NNS NNS NNS NNS NNS NNS
NNS TGG GGT CAA GGA ACA CTA

(SEQ ID NO: 9)

Oligo 1-3.
ACT GCC GTC TAT TAT TGT NNS NNS NNS NNS NNS NNS
NNS NNS TGG GGT CAA GGA ACA CTA

(SEQ ID NO: 10)

Oligo 1-4.
ACT GCC GTC TAT TAT TGT NSS NNS NNS NNS NNS NNS
NNS NNS NNS TGG GGT CAA GGA ACA CTA

(SEQ ID NO: 11)

Oligo 1-5.
ACT GCC GTC TAT TAT TGT NNS NNS NNS NNS NNS NNS
NNS NNS NNS NNS TGG GGT CAA GGA ACA CTA

(SEQ ID NO: 12)

Oligo 1-6.
ACT GCC GTC TAT TAT TGT NNS NNS NNS NNS NNS NNS
NNS NNS NNS NNS NNS TGG GGT CAA GGA ACA CTA

(SEQ ID NO: 13)

Oligo 1-7.
ACT GCC GTC TAT TAT TGT AGC NNS NNS NNS NNS NNS
NNS NNS NNS NNS NNS NNS TGG GGT CAA GGA ACA CTA

(SEQ ID NO: 14)

Oligo 1-8.
ACT GCC GTC TAT TAT TGT NNS NNS NNS NNS NNS NNS
NNS NNS NNS NNS NNS NNS NNS TGG GGT CAA GGA ACA
CTA

(SEQ ID NO: 15)

Oligo 1-9.
ACT GCC GTC TAT TAT TGT NNS NNS NNS NNS NNS NNS
NNS NNS NNS NNS NNS NNS NNS NNS TGG GGT CAA GGA
ACA CTA

(SEQ ID NO: 16)

Oligo 1-10.
ACT GCC GTC TAT TAT TGT NNS NNS NNS NNS NNS NNS
NNS NNS NNS NNS NNS NNS NNS NNS NNS TGG GGT CAA
GGA ACA CTA

(SEQ ID NO: 17)

Oligo 1-11.
ACT GCC GTC TAT TAT TGT NNS NNS NNS NNS NNS NNS
NNS NNS NNS NNS NNS NNS NNS NNS NNS NNS TGG GGT
CAA GGA ACA CTA

(SEQ ID NO: 18)

Oligo 1-12.
ACT GCC GTC TAT TAT TGT NNS NNS NNS NNS NNS NNS
NNS NNS NNS NNS NNS NNS NNS NNS NNS NNS NNS TGG
GGT CAA GGA ACA CTA.

The first mutagenic oligonucleotide (Oligo 1-1) included randomization at VH amino acid positions 35, 37, 45, and 47. The remaining oligonucleotides (Oligo 1-2 through Oligo 1-12) were permutations of the same desired sequence, in which between 7 and 17 randomized codons were included between VH amino acid positions 92 and 103 (CDR-H3). In each case, residues were hard-randomized using the NNS mixed codon set (where N corresponds to G, C, A, or T and S corresponds to G or C), as indicated in the oligonucleotide sequences above. The mutagenesis reactions were performed with all twelve of the mutagenic oligonucleotides as described previously (Sidhu et al., Meth. Enzymol. 328: 333-363 (2000)), with the exception that no uridine was used, and the helper phage used was K07M13.

The mutagenesis reactions were electroporated into E. coli strain SS320, and phage production was initiated by the addition of M13-KO7 helper phage. After overnight growth at 37° C., phage was harvested by precipitation with polyethylene glycol (PEG)/NaCl and resuspended in PBT buffer (phosphate-buffered saline (PBS) including 0.5% BSA and 0.1% Tween 20). The diversity of Library 1 was 2×10¹⁰ unique members.

C. Sorting (Affinity Selection) of VH Library 1

VH Library 1 was sorted by several rounds of stringent Protein A binding selection to identify phage expressing properly folded VH domains. Correctly folded VH domains were expected to retain the ability to bind Protein A (see FIG. 3). Ninety-six well plates (Nunc Maxisorp) were coated overnight at 4° C. with 100 μL Protein A (10 μg/ml) per well and blocked for one hour with 200 μL/well of PBS containing 0.5% BSA at room temperature. Phage solution from Library 1 was added to the coated immunoplates (100 μL per well of 10¹² pfu/mL solution). Following a two hour incubation at room temperature to permit phage binding, the plates were washed ten times with PBST buffer (PBS containing 0.05% Tween 20).

Bound phage was eluted from each well with 100 μL 0.1 M HCl for five minutes and the eluants from each well were neutralized with 15 μL 1.0 M Tris base pH 11.0. The eluted phage were further amplified in E. coli XL 1-blue cells with the addition of M13-KO7 helper phage (New England Biolabs). The amplified phage were used for further rounds of selection. The amplified phage libraries were cycled through four additional rounds of affinity plate selection against Protein A.

After the fifth round of Protein A selection, the amplified Library 1 VH domains were sorted based on their abilities to bind to an anti-pentahistidine tag (SEQ ID NO: 273) antibody (Qiagen). E. coli CJ236 cells (100 μL) were incubated with 10 μL of the phage library pool from the fifth round of Protein A sorting for 20 minutes at 37° C. with agitation. The infection mixture was spread on a large carbenicillin Petri dish and incubated overnight at 37° C. The bacterial layer was resuspended in about 15 mL of 2YT buffer containing carbenicillin and chloramphenicol at the surface of the petri dish. The solution was removed from the dish and 30 μL of a 10¹¹ pfu/mL solution of M13-K07 helper phage was added, followed by incubation at 37° C. for one hour with agitation. One milliliter of the bacteria/phage mixture was transferred to about 250 mL 2YT buffer containing carbenicillin and kanamycin, and incubated overnight at 37° C. with agitation. DNA was purified and a small-scale Kunkel mutagenesis was performed as described above to introduce a hexahistidine tag (SEQ ID NO: 274) and amber stop codon into the library. The mutagenic oligonucleotide used was: TCCTCGAGTGGCGGTGGCCACCATCACCATCACCATTAGTCTGGTTCCGGTGATT TT (SEQ ID NO: 19). The products of the mutagenesis reaction were electroporated into E. coli XL-1 blue cells, and a library was constructed as above. A selection was performed against anti-pentahistidine tag (SEQ ID NO: 273) antibody (Qiagen) (100 μL/well of a 5 μg/mL solution). After the hexahistidine (SEQ ID NO: 274) selection and amplification, one final round of Protein A sorting was performed under the same conditions described above.

D. Sequencing and VH Domain Analysis

Individual clones from the seventh round of selection for Library 1 were grown overnight in a 96 well format at 37° C. in 400 μL of 2YT broth supplemented with carbenicillin and M13-KO7 helper phage. Culture supernatants containing phage particles were used as templates for PCR reactions to amplify the DNA fragment encoding the VH domain. PCR primers were designed to add M13F and M13R universal sequencing primers at either end of the amplified fragment, thus allowing the M13F and M13R primers to be used in sequencing reactions. The forward PCR primer sequence was TGTAAAACGACGGCCAGTCACACAGGAAACAGCCAG (SEQ ID NO: 20) and the reverse PCR primer sequence was CAGGAAACAGCTATGACCGTAATCAGTAGCGACAGA (SEQ ID NO: 21). Amplified DNA fragments were sequenced using big-dye terminator sequencing reactions using standard methodologies. The sequencing reactions were analyzed on an ABI Prism 3700 96-capillary DNA analyzer (PE Biosystems, Foster City, Calif.). All reactions were performed in a 96-well format.

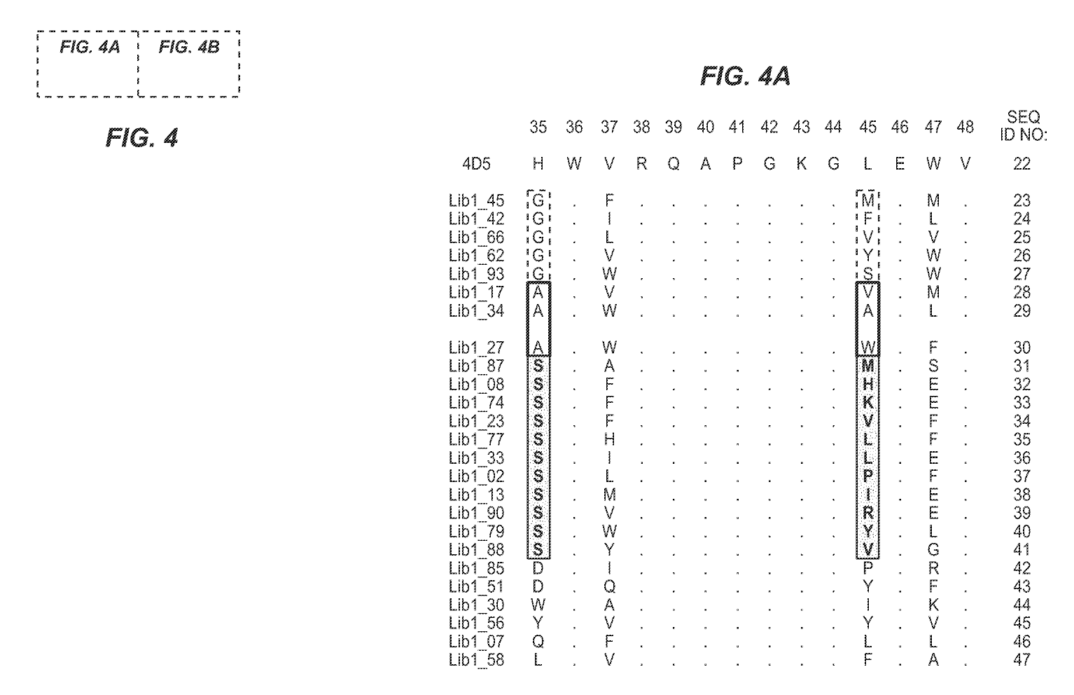

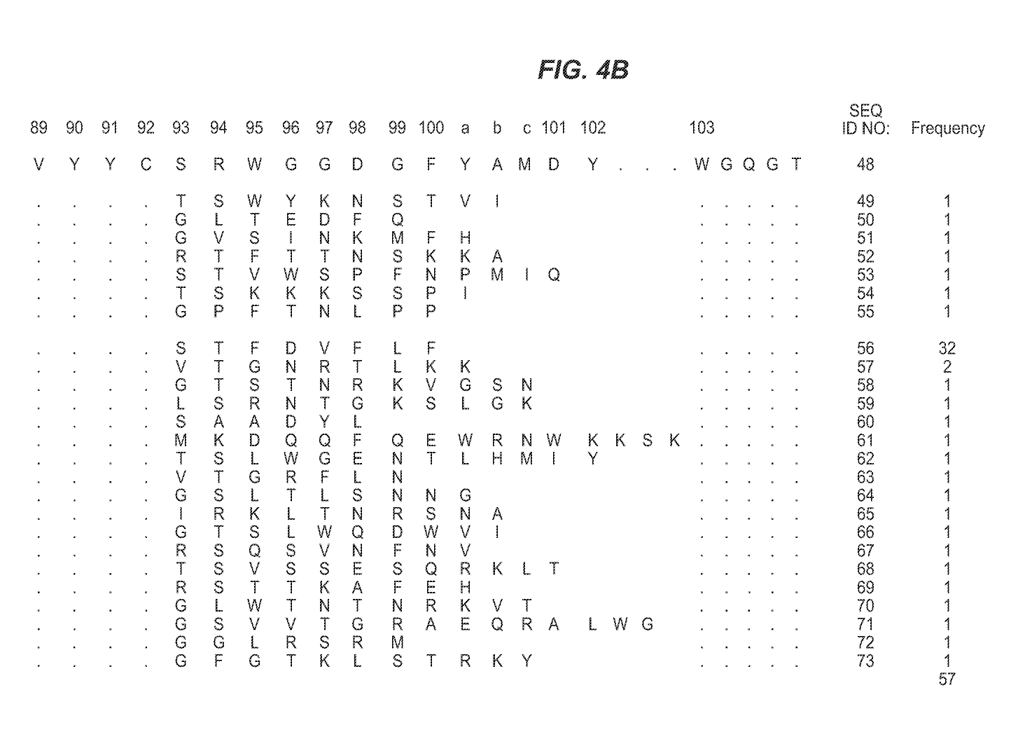

Of the 100 clones that were sequenced, 57 readable sequences were obtained. Of those 57 sequences, 25 were unique and are set forth in FIGS. 4A and 4B. No consensus sequence was observable in CDR-H3. Moreover, there was no clear preference in CDR-H3 length among the selected VH domains. Several general trends were observed in the sequence results regarding the residues along the former VH-VL interface. First, there was a clear preference for small residues at position 35, such as glycine, alanine, and serine. Second, positions 37 and 45 were predominantly hydrophobic (i.e., tryptophan, phenylalanine, and tyrosine). Third, position 47 appeared to depend on the residue at position 35. For example, when a glycine or alanine was found at position 35, position 47 was occupied by a bulky hydrophobic residue such as tryptophan or methionine. In contrast, when position 35 was a serine, position 47 was occupied by glutamate or phenylalanine.

Protein A selection of phage-displayed VH domains served as a useful tool to select for proteins that are potentially well expressed in E. coli because the process of displaying a protein on the surface of phage particles is similar to the process for expression of a protein in E. coli. Thus, if a VH domain was sufficiently stable to be expressed on phage, it would likely be well expressed in E. coli. However, further characterization of the VH domain selectants from Library 1 was necessary to clearly establish the VH domain as correctly folded and truly stable. Sixteen of the twenty-five identified unique sequences were selected for further analysis based on their frequency among the 100 examined clones and their sequences. A three-step screening strategy was used for each protein to (a) measure the Protein A binding ability of protein expressed in E. coli; (b) examine the tendency to aggregate; and (c) assess thermal stability.

1. VH Domain Expression

Each of the sixteen selected VH domains were expressed in E. coli as a soluble protein and the resulting cell lysates were analyzed by chromatography on columns containing Protein A-coupled resin. Properly folded VH domains should bind more tightly to Protein A than non-correctly folded domains. Consequently, the yield of a particular VH domain that specifically bound to Protein A should be indicative of the degree to which that domain was correctly folded.

To allow the purification of soluble VH domains in non-suppressor bacterial strains, the phagemids were modified by the introduction of an amber stop codon just before the P3C open reading frame. Individual VH domains were expressed in E. coli BL21 cells (Stratagene, La Jolla, Calif.) in 500 mL shake flask culture by induction with 0.4 mM IPTG for three hours. Frozen cell pellets were resolubilized in 100 mL 25 mM Tris, 25 mM NaCl, 5 mM EDTA pH 7. 1. After homogenization with a cell homogenizer (Ultra-Turrax T8, IKA Labortechnik, Staufen, Germany), the cells were lysed in an M-110F Microfluidizer Processor (Microfluidics, MA). The cell lysate was centrifuged for 30 minutes at 8,000 RPM at 4° C. The supernatant was filtered through a 20 μm filter and loaded onto a 2 mL Protein A-sepharose column for gravity-driven chromatography. After washing the column with at least 20 mL of 10 mM Tris, 1 mM EDTA, pH 8.0, each VH domain was eluted with 0.1 M glycine pH 3.0. Four 2.5 mL fractions were collected, and the eluants were neutralized with 0.5 mL 1 M Tris pH 8.0. Protein concentrations were determined using amino acid composition analysis, a Bradford assay, or absorbance at 280 nm using extinction coefficients calculated based on the amino acid sequence of the particular VH domain.

The wild-type 4D5 VH domain was Protein A-purified at a yield of approximately 2 mg/L. Six clones were identified that had a yield at least 4-fold higher than the wild-type 4D5 VH domain, as shown in FIG. 5 and Table 2. Only those six clones were further characterized.

2. Analysis of VH Domain Oligomeric State

Isolated VH domains with minimal tendency to aggregate are preferred both for library construction and for therapeutic use. Aggregation may interfere with the ability of the domain to interact with its target antigen and may be an indicator of improper folding. The oligomeric state of the six clones with the highest yields in the Protein A chromatography assay was determined by gel filtration chromatography and light scattering analysis.

Molar mass determination was performed by light scattering using an Agilent 1100 series HPLC system (Agilent, Palo Alto, Calif.) in line with a Wyatt MiniDawn Multiangle Light Scattering detector (Wyatt Technology, Santa Barbera, Calif.). Concentration measurements were made using an online Wyatt OPTILA DSP interferometric refractometer (Wyatt Technology, Santa Barbera, Calif.). Astra software (Wyatt Technology) was used for light scattering data acquisition and processing. The temperature of the light scattering unit was maintained at 25° C. and the temperature of the refractometer was kept at 35° C. The column and all external connections were maintained at room temperature. A value of 0.185 mL/g was assumed for the dn/dc ratio of the protein. The signal from monomeric BSA normalized the detector responses.

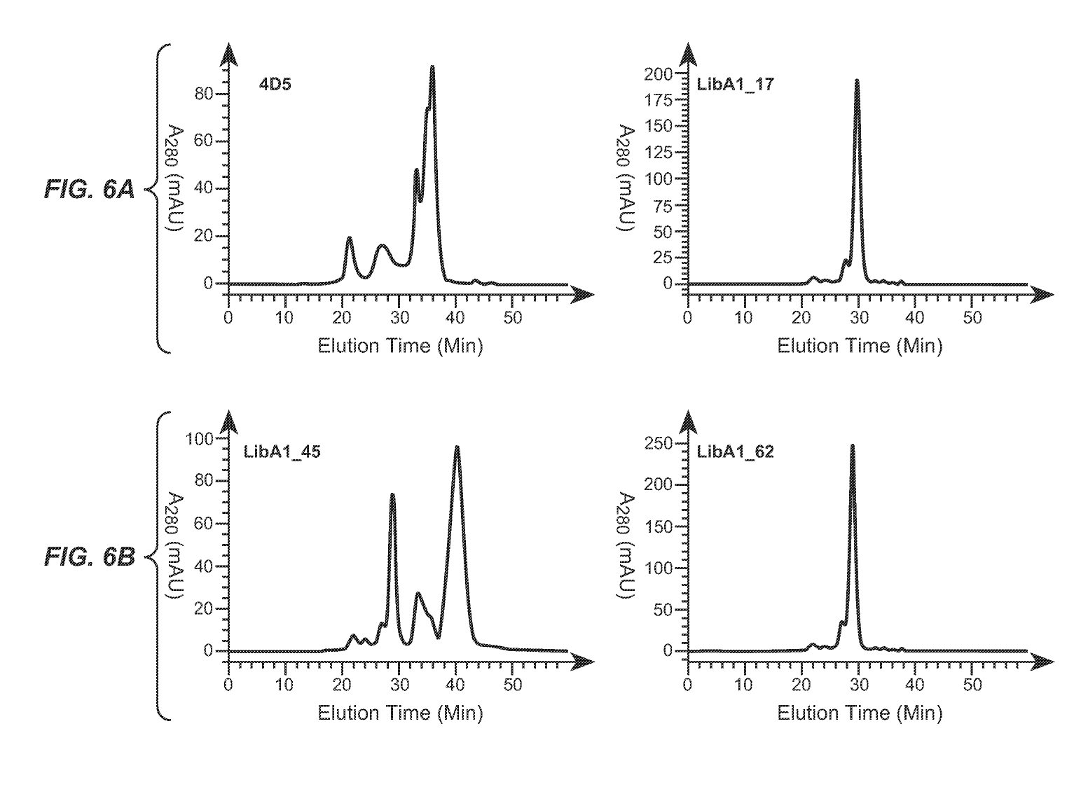

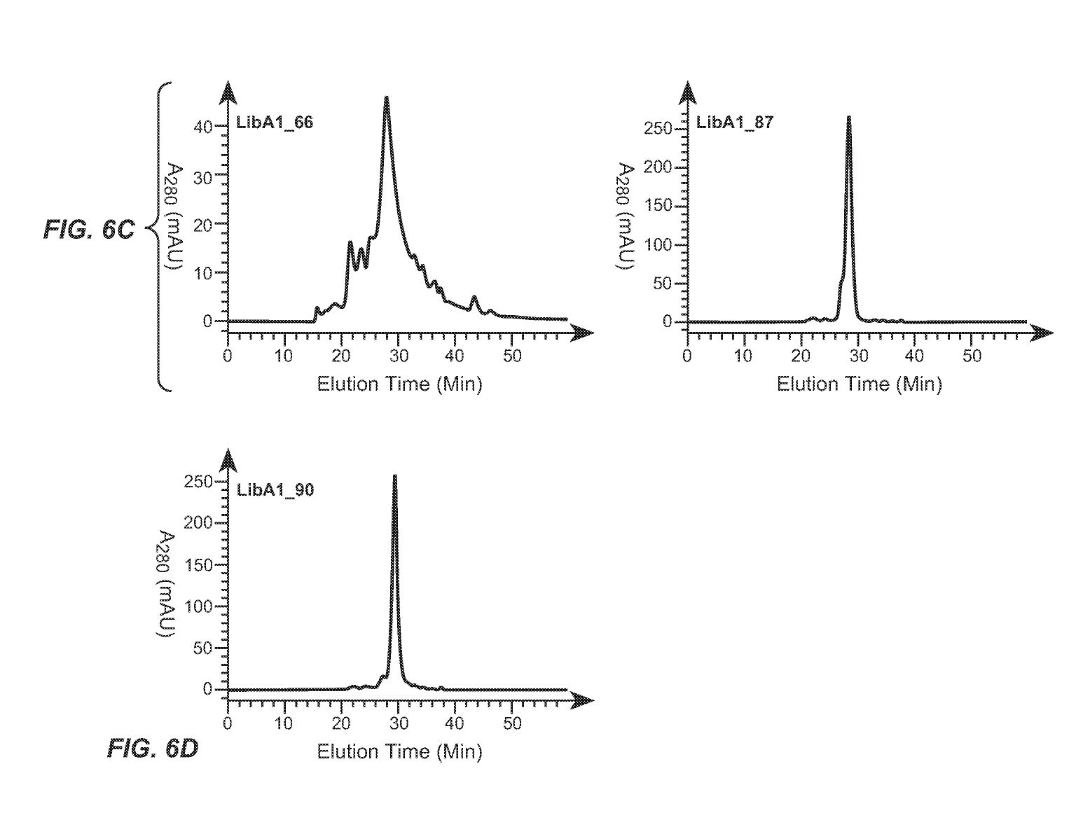

VH domain samples (100 μL of an approximately 1 mg/mL solution) were loaded onto a Superdex 75 HR 10/30 column (Amersham Biosciences) at a flow rate of 0.5 mL/min. The mobile phase was filtered PBS pH 7.2 containing 0.5 M NaCl. Protein concentrations were determined using amino acid composition analysis, a Bradford assay, or absorbance at 280 nm using extinction coefficients calculated based on the sequence of the VH domain. The results are shown in FIGS. 6A-6D and Table 2. The wild-type VH domain was retained on the column for an extended period and did not elute as expected based on its molecular weight. It eluted from the column in several peaks, and about 50% of the wild-type VH domain protein was aggregated, as estimated by light scattering analysis (see FIG. 6A and Table 2). Four of the six variant VH domains (clones Lib1_{—}17, Lib1_{—}62, Lib1_{—}87, and Lib1_{—}90) were essentially monomeric as determined by light scattering, and had similar retention times on the column to that of the wild-type 4D5 VH domain. All of the isolated VH domains had a recovery of close to 100%.

3. Analysis of VH Domain Thermal Stability

The thermal stability of the six VH domains was assessed by measuring the melting temperature of each protein. The Tₘ reflects the stability of folding, as does the melting curve. Thermal stabilities of the purified VH domain proteins were measured using a Jasco spectrometer model J-810 (Jasco, Easton, Md.). Purified VH domains were diluted to 10 μM in PBS. Unfolding of the proteins was monitored at 207 nm over a range of temperatures from 25° C. to 85° C. at 5 degree intervals. Melting temperatures were determined for both the unfolding and refolding transitions.

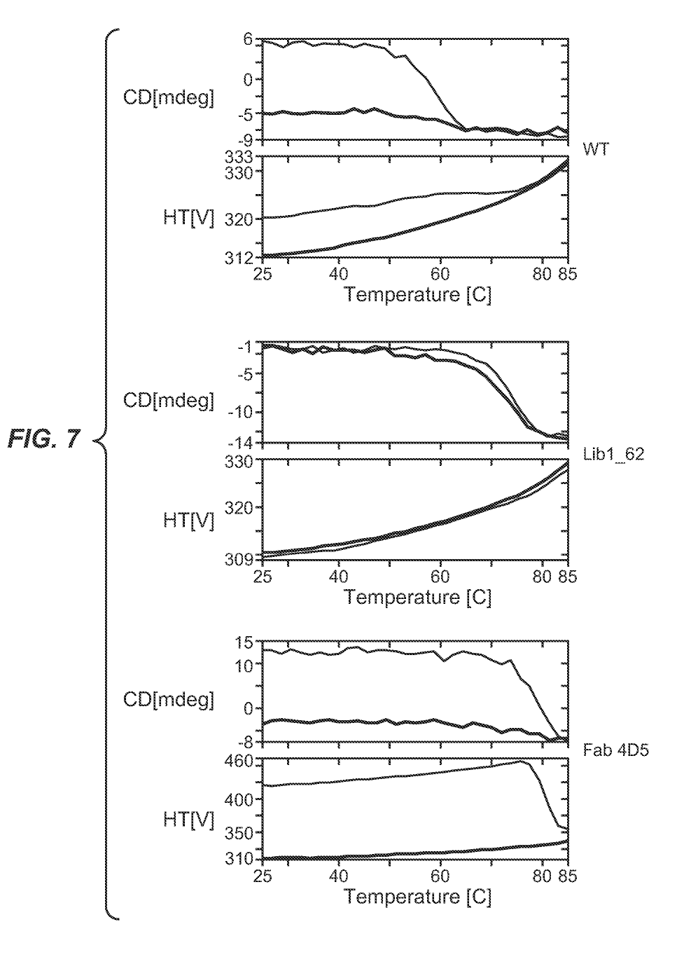

All six VH domain variants had Tₘ greater than the wild-type 4D5 Tₘ (FIG. 7 and Table 2). A Fab version of 4D5 served as a positive control, and had a Tₘ of 80° C. and irreversible folding, as expected. Only three of the six Library 1 VH domains had fully reversible melting curves: Lib1_{—}62, Lib1_{—}87, and Lib1_{—}90 (see FIG. 7). Lib1_{—}62 had a Tₘ of 73° C., the highest among all of the variants, and significantly higher than the wild-type 4D5 VH domain Tₘ.

TABLE 2
Properties of certain library selectants

| | | Calcu- | Appar- | | | |
|---|---|---|---|---|---|---|
| | | lated | ent | | | Revers- |
| | Yield | Mw | Mw | Aggregate | Tm | ible |
| Clone | (mg/L) | (Dalton) | (Dalton) | (%) | (° C.) | folding? |
| 4D5 (WT) | 2 | 14386 | 14386 | ND* | 55 | No |
| Lib1_17 | 10 | 13701 | 14210 | 13 | 70 | No |
| Lib1_45 | 13 | 13990 | 15640 | 40 | 75 | No |
| Lib1_62 | 14 | 13984 | 14630 | 15 | 75 | Yes |
| Lib1_66 | 6 | 13726 | 24400 | No | 73 | No |
| | | | | monomer | | |
| Lib1_87 | 8 | 13718 | 14180 | 2 | 65 | Yes |
| Lib1_90 | 7 | 13969 | 14540 | 8 | 67 | Yes |
| Lib2_3 | 17 | 13805 | 15190 | 12 | 75 | Yes |
| Lib2_3.4D5H3 | 11 | 14124 | 14450 | 5 | 80 | Yes |
| Lib2_3.T57E | 3 | 13833 | 14090 | 5 | 73 | Yes |

*ND: not determined

E. ELISA Binding Assays

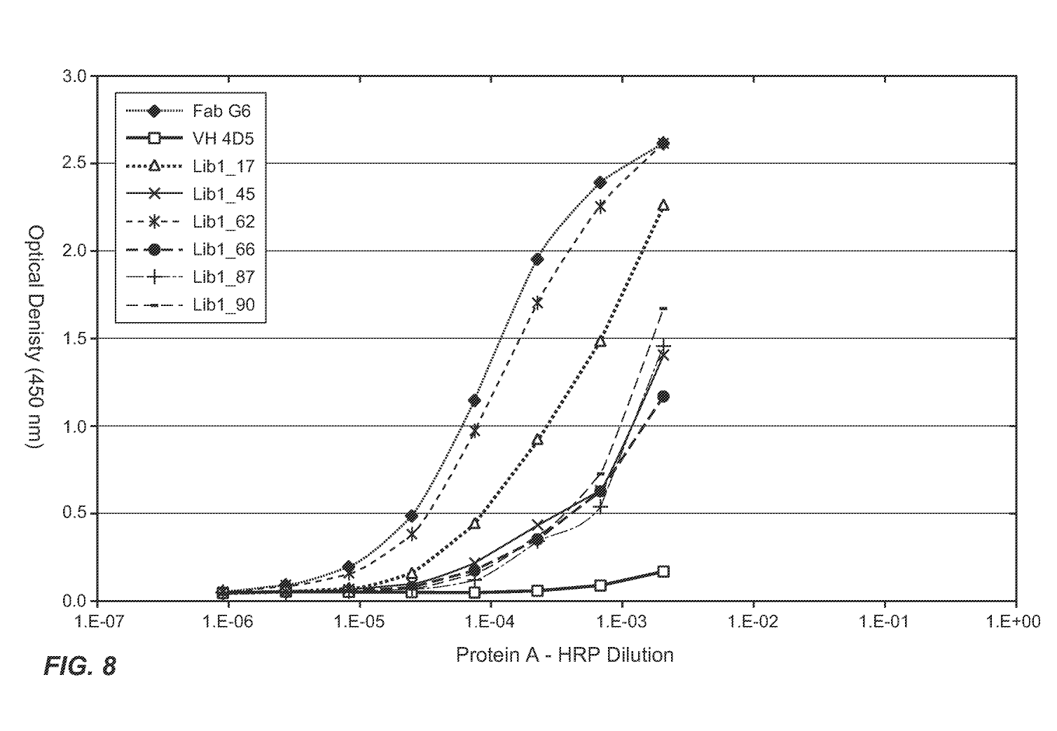

Nunc 96-well Maxisorp immunoplates were coated overnight at 4° C. with 10 μg/mL of each VH domain protein. The wells were blocked with BSA for one hour at room temperature. Three-fold serial dilutions of horseradish peroxidase (HRP) conjugated Protein A (Zymed laboratories, South San Francisco, Calif.) were added to the coated and blocked immunoplates and incubated for two hours to permit Protein A binding to immobilized VH domains. The plates were washed eight times with PBS containing 0.05% Tween 20. Binding was visualized by the addition of the HRP substrate 3,3′-5,5′-tetramethylbenzidine/H₂O₂ peroxidase (TMB) (Kirkegaard & Perry Laboratories Inc., Gaithersburg, Md., USA) for five minutes. The reaction was stopped with 1.0 M H₃PO₄, and the plates were read spectrophotometrically at 450 nm using the Multiskan Ascent microtiter plate reader (Thermo Labsystems, Vantaa, Finland). The results are shown in FIG. 8. Fab 4D5 bound best to Protein A, but Lib1_{—}62 and Lib1_{—}90 both bound Protein A almost as well and better than the binding observed between the wild-type 4D5 VH domain and Protein A.

### Example 2

### Construction, Sorting, and Analysis of Phage-Displayed VH Domain Library 2

Of the six clones from Library 1 analyzed in depth, VH domain Lib1_{—}62 had the most useful combination of characteristics for library construction purposes. Lib1_{—}62 was essentially monomeric in solution, expressed well in bacteria, and had a high Tₘ, with a fully reversible melting curve. Furthermore, it had a high yield in Protein A chromatography assays. These results suggested that the Lib1_{—}62 protein was correctly folded and did not aggregate significantly. Notably, Lib1_{—}62 had only two framework amino acid differences from the wild-type 4D5 VH domain framework amino acid sequence: a glycine at position 37 and a tyrosine at position 55. Modifications were made to the Lib1_{—}62 sequence to ascertain whether the conformational stability of the Lib1_{—}62 VH domain could be further enhanced.

Construction of the second library involved randomizing residues located in the central VL-contacting interface of the VH domain, specifically those predicted to have 20 A² of their surface normally buried by the VL domain. Those residues included Q39, G44, R50, Y91, W103, and Q105. CDR-H3 was also randomized at certain positions between 92 through 104, but without length variation. Additionally, the residues that had been randomized in Library 1 (positions 35, 37, 45, and 47) were again randomized. Given that the Lib1_{—}62 VH domain was already stable, only soft-randomization was employed at each of the randomized positions. A soft-randomization strategy maintained a bias against the wild-type sequence while introducing a 50% mutation rate at each selected position. Using soft-randomization, mutations would be present in the selectants only if they were critical for domain stabilization.

The method for library construction was identical to that for Library 1 (see Example 1B), and used the same stop template as that used in the construction of Library 1. The oligonucleotides used for the Library 2 mutagenesis reaction were:

(SEQ ID NO: 74)

Oligo 2-1.
ATT AAA GAC ACC TAT ATA 667 TGG 687 CGT 756 GCC
CCG GGT AAG 667 857 GAA 866 GTT GCA 566 ATT TAT
CCT ACG AAT GGT

(SEQ ID NO: 75)

Oligo 2-2.
GAG GAC ACT GCC GTC TAT 858 TGC 565 576 888 575
576 558 877 556 555 678 866 GGT 755 GGA ACA CTA
GTC ACC GTC

The numerical positions in the sequences for Oligo 2-1 and 2-2 indicate that certain nucleotide positions were 70% of the time occupied by the indicated base and 10% of the time occupied by each one of the other three bases. Where such soft randomization was included at a particular base, the presence of the soft randomization is indicated by the presence of a number at that base position. The number “5” indicates that the base adenine is present 70% of the time at that position, while the bases guanine, cytosine, and thymine are each present 10% of the time. Similarly, the number “6” refers to guanine, “7” to cytosine, and “8” to thymine, where in each case, each of the other three bases is present only 10% of the time. The first mutagenic oligonucleotide set based on Oligo 2-1 included soft randomization at VH amino acid positions 35, 37, 39, 44, 45, 47, and 50. The second mutagenic oligonucleotide set based on Oligo 2-2 included soft randomization at VH amino acid positions 91, 93-103, and 105.

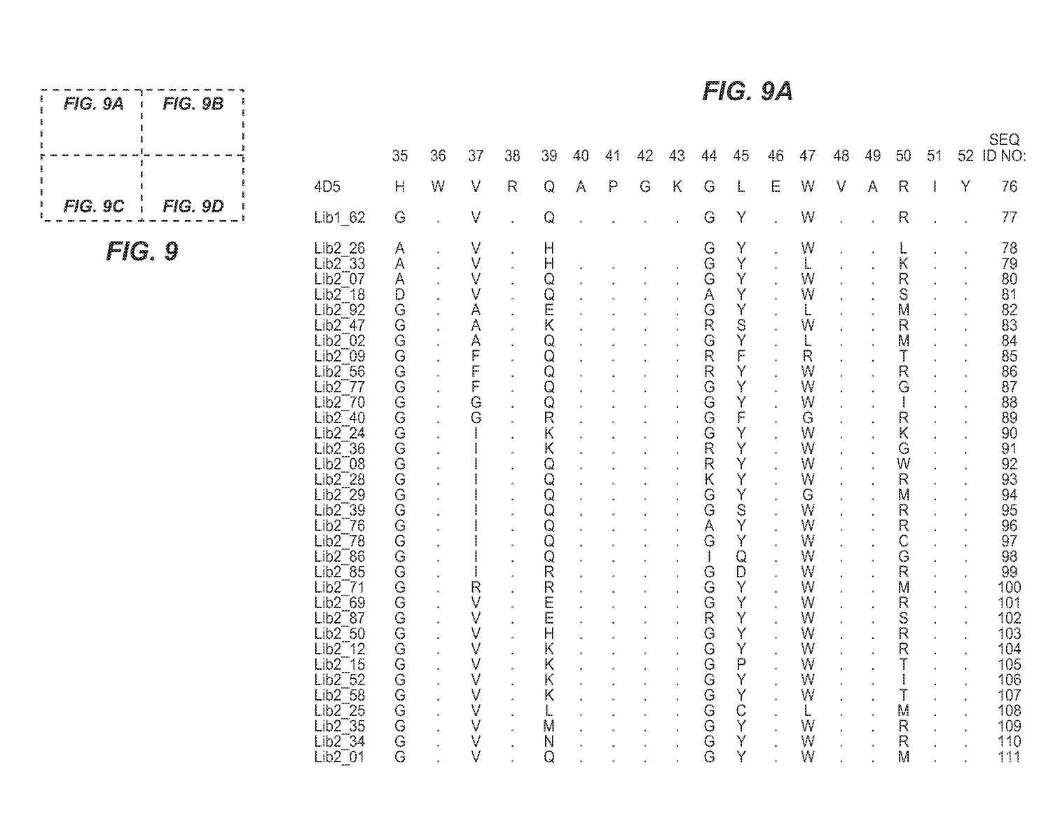

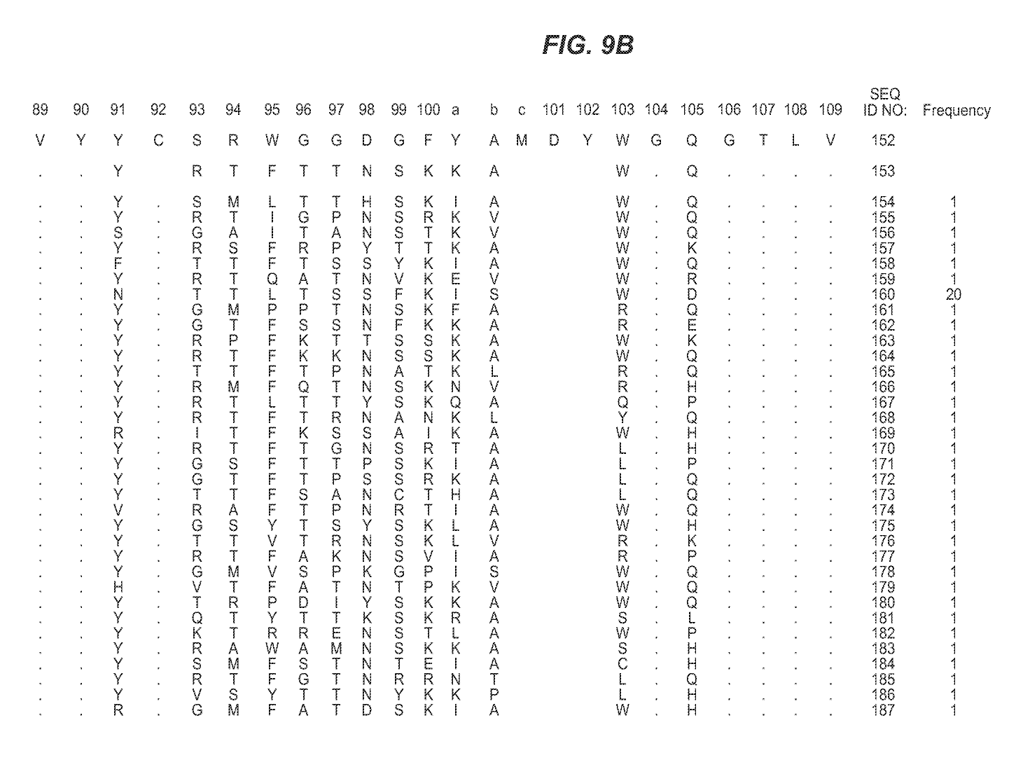

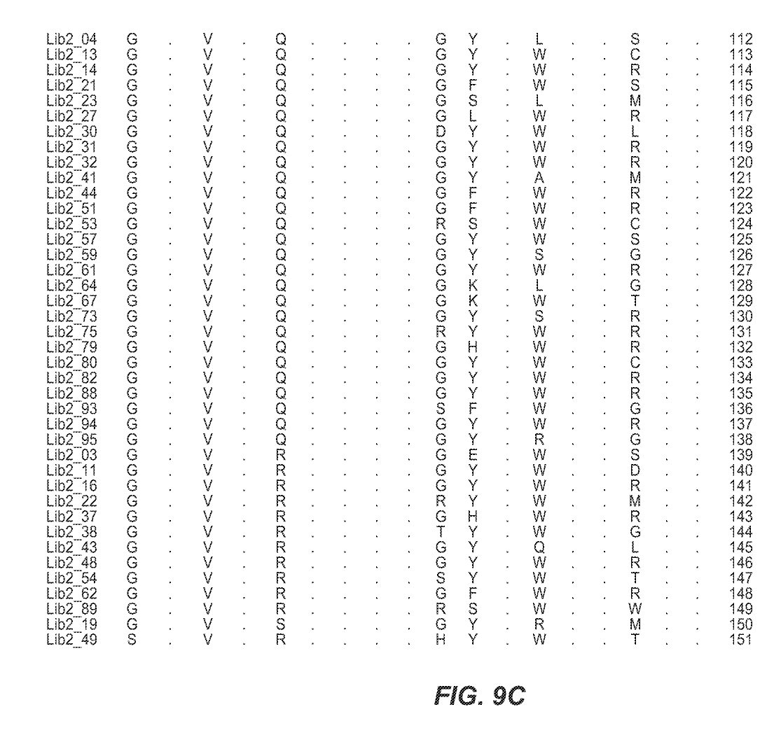

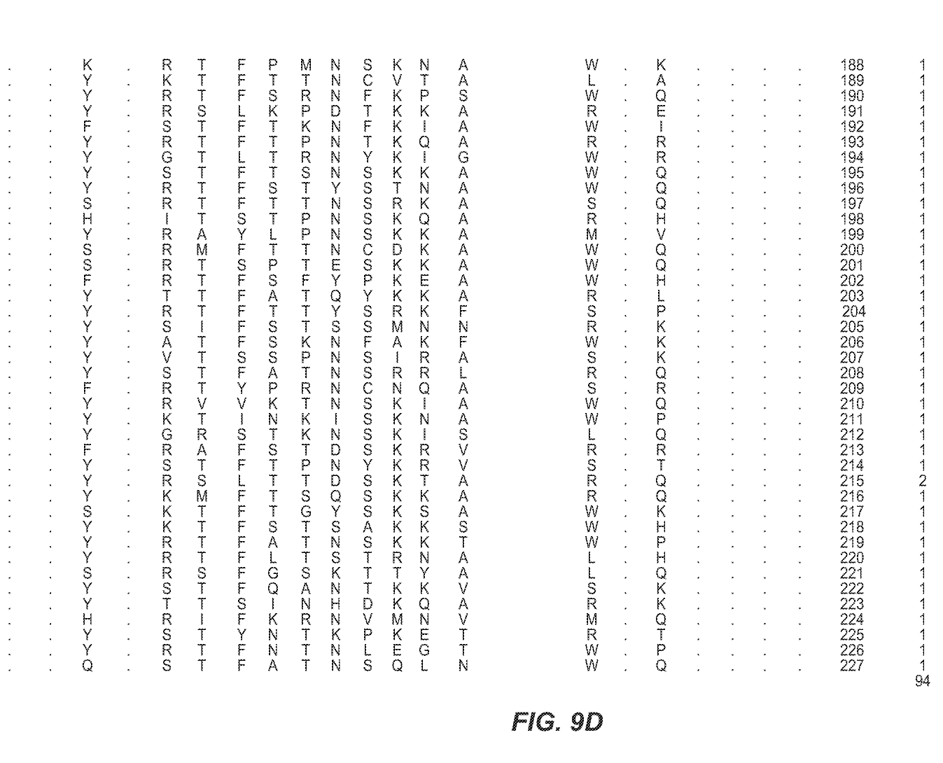

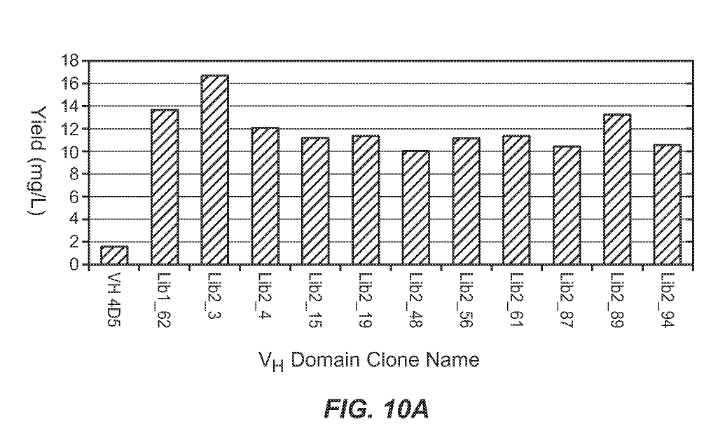

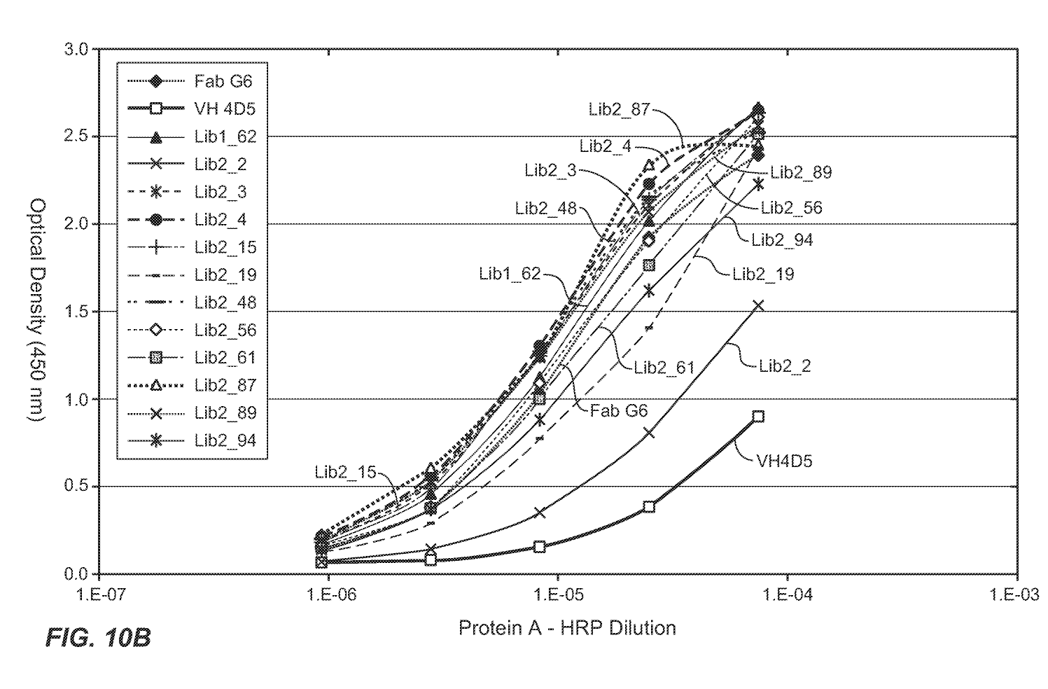

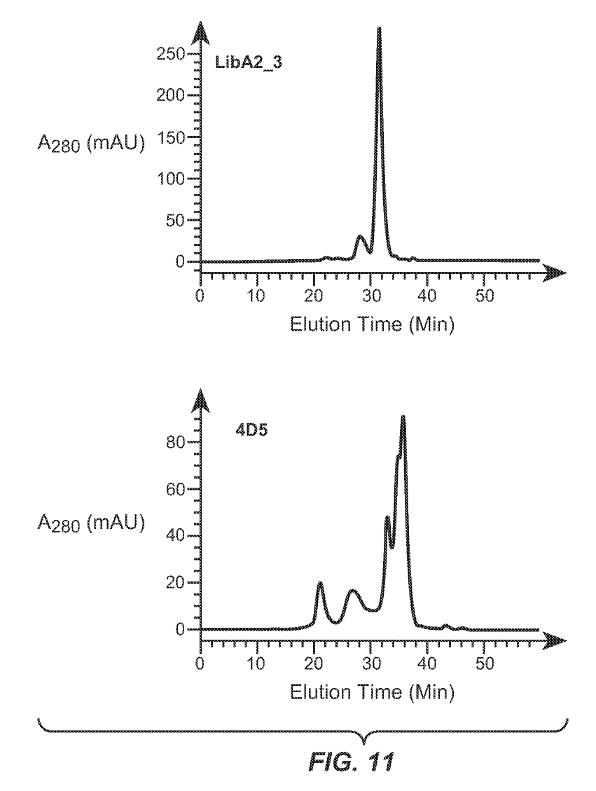

Library 2 was sorted through seven rounds of affinity plate selection against Protein A to enrich for library members that were likely to be properly folded. The methodology used was identical to that used for Library 1 (see Example 1C). Further, the stringency of the selection was increased in two ways. First, the phage solution was heated at 50° C. prior to panning. Second, the number of washes was increased to 15. After selection, 100 clones were sequenced, using the same methodology and primers as described in Example 1D. Seventy-seven readable sequences were obtained, of which 74 were unique (FIGS. 9A-9D). More than 95% of the unique sequences had a glycine at position 35, identical to the parent sequence Lib1_{—}62. Forty-four of the seventy-four unique sequences were selected for further analysis based on the frequency of their occurrence in the seventy-seven readable sequences and their amino acid sequences. Those forty-four proteins were further characterized by the same screening strategy used to characterize Library 1 (see Examples 1D and 1E). Nine of the clones had an equal or higher yield to that of the Lib1_{—}62 VH domain in protein A chromatography according to Example 1D(a) (FIG. 10A). Clone Lib2_{—}3 had a variable yield of up to 17 mg/L, which was about 1.7 times greater than that of Lib1_{—}62. As a qualitative measure of the interaction of each VH domain with Protein A, ELISA assays were performed according to the methodology described in Example 1E. As shown in FIG. 10B, Lib2_{—}2, Lib2_{—}19, and Lib2_{—}94 bound less well to Protein A than the other eight Library 2 clones, which were similar to Lib1_{—}62 in terms of Protein A binding. Due to its significantly higher yield and specific binding to Protein A, clone Lib2_{—}3 was selected for further analysis.

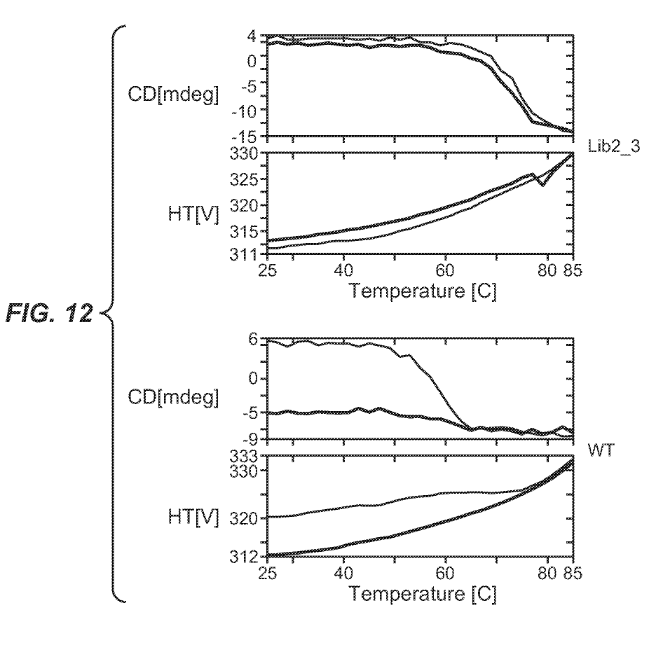

The purified Lib2_{—}3 VH domain was subjected to size-exclusion chromatography and light scattering analysis as described in Example 1D(b) and thermal stability analysis as described in Example 1D(c). The LibA2_{—}3 VH domain was essentially monomeric (FIG. 11, as compared to LibA1_{—}62 curve in FIG. 6B). The determined melting curve was fully reversible and indicated a Tm of about 73° C., similar to that of LibA1_{—}62 (compare FIG. 12 to FIG. 7 and Table 2).

The sequences of the Lib1_{—}62 and Lib2_{—}3 VH domains differ at three positions. In Lib1_{—}62, position 39 was glutamine, position 45 was tyrosine, and position 50 is arginine. In Lib2_{—}3, position 39 was arginine, position 45 was glutamic acid, and position 50 was serine. In both sequences, position 35 remained glycine while position 47 remained tryptophan. Positions 39, 45, and 50 are located in the region of VH known to interface with VL, and according to the crystal structure of 4D5, protrude into the VL layer. The increase in folding stability between Lib1_{—}62 and Lib2_{—}3 (as evidenced by substantially increased yield in the Protein A chromatography assay) observed upon replacement of positions 39, 45, and 50 with hydrophilic residues suggested that increasing the hydrophilic character of the VH-VL interface region improved the stability of the isolated VH domain.

### Example 3

### Lead Candidate Framework Shotgun-Scanning Analyses

While Library 2 was constructed to allow soft randomization at positions 35, 37, 39, 44, 45, 47, 50, and 91 (as well as CDR-H3), the Lib2_{—}3 VH domain sequence contained modified residues only at positions 35, 39, 45, and 50 and had wild-type residues at positions 37, 44, 47, and 91. Two further libraries were constructed using Lib2_{—}3 as a starting scaffold to observe any general trends in sequence conservation among correctly folded domains.

a. Construction of Library 3

Library 3 was constructed to keep constant the VH-VL interface positions in Lib2_{—}3 that were identical to the wild-type 4D5 VH sequence (positions V37, G44, W47, and Y91) while hard-randomizing those interface positions that had varied from the wild-type 4D5 sequence (positions G35, R39, E45, and S50). The method for library construction was identical to that for Library 1 (see Example 1B), and used the same stop template as that used in the construction of Library 1. The oligonucleotides used for the Library 3 mutagenesis reaction were:

(SEQ ID NO: 228)

Oligo 3-1.
ATT AAA GAC ACC TAT ATA NNK TGG GTC CGT NNK GCC
CCG GGT AAG GGC NNK GAA TGG GTT GCA NNK ATT TAT
CCT ACG AAT GGT

(SEQ ID NO: 229)

Oligo 3-2.
ACT GCC GTC TAT TAT TGT AGA TCG CTT ACA ACA GAT
TCC AAG ACA GCT CGA GGT CAA GGA ACA CTA GTC

Hard-randomizations were performed using the NNK mixed codon set (where N corresponds to G, C, A, or T and K corresponds to G or T), as indicated in the oligonucleotide sequences above.

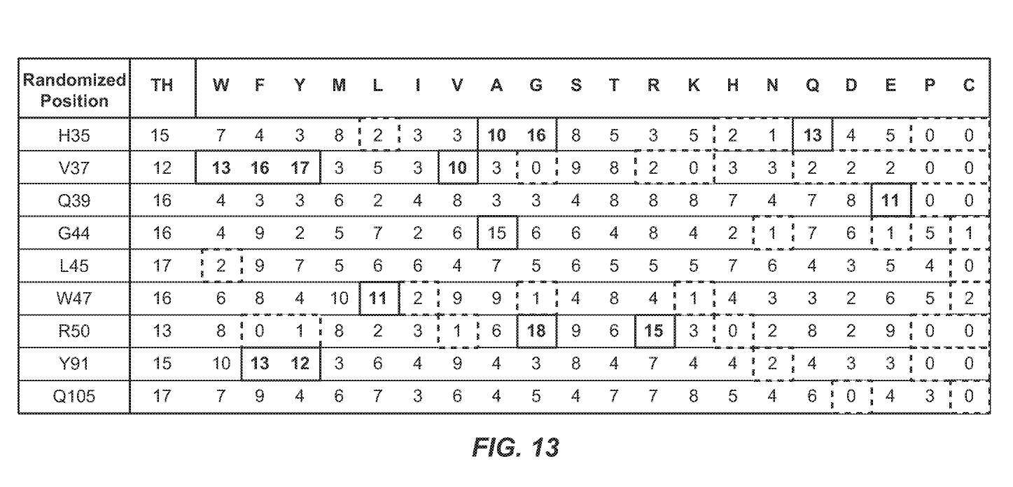

Library 3 was cycled through two rounds of affinity plate selection against Protein A to enrich for properly folded library members. The methodology used was similar to that used for Library 1 (see Example 1C), but without an additional selection for binding to an anti-hexahistidine (SEQ ID NO: 274) antibody. After selection, 200 clones were selected for sequencing, using the same methodology and primers as described in Example 1D. The unique sequences were aligned and the occurrence of each amino acid at each randomized position was tabulated. The totals were normalized by dividing them by the number of times each amino acid was encoded by the redundant NNK codon. The normalized percentages at each randomized position are shown in FIG. 13.

When positions V37, G44, W47, and Y91 were kept constant, position 35 was biased towards a small aliphatic residue such as glycine or alanine. Serine and glutamine were also well tolerated. Serine at position 35 had also been observed in Library 1 (see FIGS. 4A and 4B). Thus, when tryptophan was present at position 47, a small residue at position 35 appeared to be important for proper folding of the VH domain. Position 39 was largely random with a slight preference for glutamate, and position 45 was fully random. Position 50 had a preference for glycine and arginine. Glutamine is a neutral hydrophilic residue, and arginine is a charged polar residue, both of which may serve to further increase the hydrophilicity of the VH-VL interface region of the VH domain.

b. Construction of Library 4

Library 4 was constructed to hard-randomize the VH-VL interface positions in Lib2_{—}3 that were identical to the wild-type 4D5 VH sequence (positions V37, G44, W47, and Y91) while keeping constant those interface positions that had varied from the wild-type 4D5 sequence (positions G35, R39, E45, and S50). Position 105 was also randomized, as in Library 2. The method for library construction was identical to that for Library 1 (see Example 1B). The oligonucleotides used for the Library 4 mutagenesis reaction were:

(SEQ ID NO: 230)

Oligo 4-1.
ATT AAA GAC ACC TAT ATA GGA TGG NNK CGT CGG GCC
CCG GGT AAG NNK GAG GAA NNK GTT GCA AGT ATT TAT
CCT ACG AAT GGT

(SEQ ID NO: 231)

Oligo 4-2.
GAG GAC ACT GCC GTC TAT NNK TGT AGA TCG CTT ACA
ACA GAT TCC AAG ACA GCT CGA GGT NNK GGA ACA CTA
GTC ACC GTC

Hard-randomizations were performed using the NNK mixed codon set (where N corresponds to G, C, A, or T and K corresponds to G or T), as indicated in the oligonucleotide sequences above.

Library 4 was cycled through two rounds of affinity plate selection against Protein A to enrich for properly folded library members. The methodology used was similar to that used for Library 1 (see Example 1C), but without an additional selection for binding to an anti-hexahistidine (SEQ ID NO: 274) antibody. After selection, 200 clones were selected for sequencing, using the same methodology and primers as described in Example 1D. The unique sequences were aligned and the occurrence of each amino acid at each randomized position was tabulated. The totals were normalized by dividing them by the number of times each amino acid was encoded by the redundant NNK codon. The normalized percentages at each randomized position are shown in FIG. 13.

When positions G35, R39, L45, and S50 were kept constant, hydrophobic residues were clearly preferred at positions 37 and 91. Small residues such as alanine were preferred at position 44. Position 47 was random, but small aliphatic residues like leucine, valine, and alanine were better tolerated than tryptophan at that position. In fact, a charged residue such as glutamate occurred at the same frequency as tryptophan at that position. Notably, glutamate also appeared at this position with some frequency in Library 1 (see FIGS. 4A and 4B).

### Example 4

### Further Analysis of VH Domain Position 35/47 Mutants

The results from Libraries 3 and 4 illustrated that a small residue like alanine, glycine, or serine is necessary at position 35 of the isolated VH domain when a large, bulky hydrophobic residue like tryptophan is present at position 47. One rationale for the pairing of a glycine at position 35 with the wild-type tryptophan at position 47 was provided by Jespers et al., where a crystal structure of such a mutant VH domain showed that the side-chain of the tryptophan fit into a cavity created by the glycine at position 35 (Jespers et al., J. Mol. Biol. 337: 893-903 (2004)). The present data also showed that glycine was not tolerated at position 47, unlike the camelid molecules, in accord with previous findings where a glycine substitution at position 47 reduced the tendency of the camelized domains to aggregate but the modified domains were still poorly expressed and less thermodynamically stable than their wild-type counterparts (Davies et al., Biotechnology (1995) 13: 475-479). However, the data from Libraries 3 and 4 also surprisingly indicated that other amino acids aside from tryptophan were well-tolerated at position 47 when position 35 was a glycine, and may even have been better tolerated than tryptophan itself. Furthermore, the data showed that position 35 did not have to be a glycine if the residue at position 47 was modified. For example, a combination of S35/E47 had been conserved in a significant number of sequences from Library 1, and the statistical analysis of Libraries 3 and 4 confirmed the bias for those residues at positions 35 and 47.

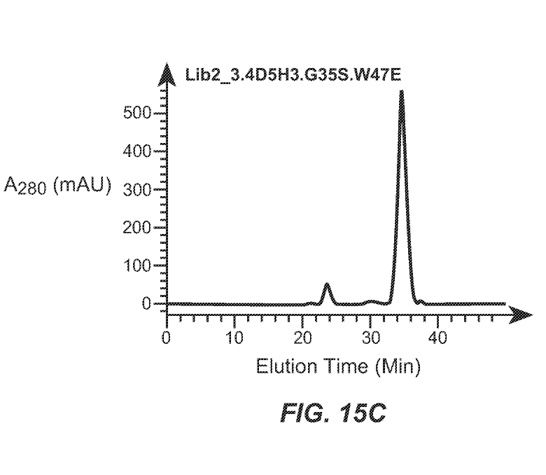

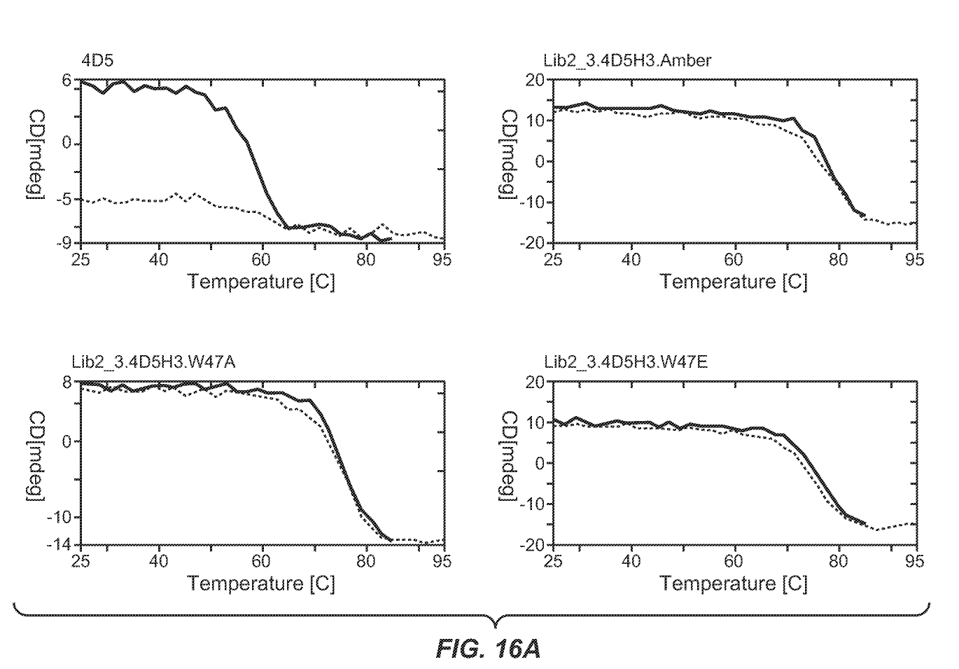

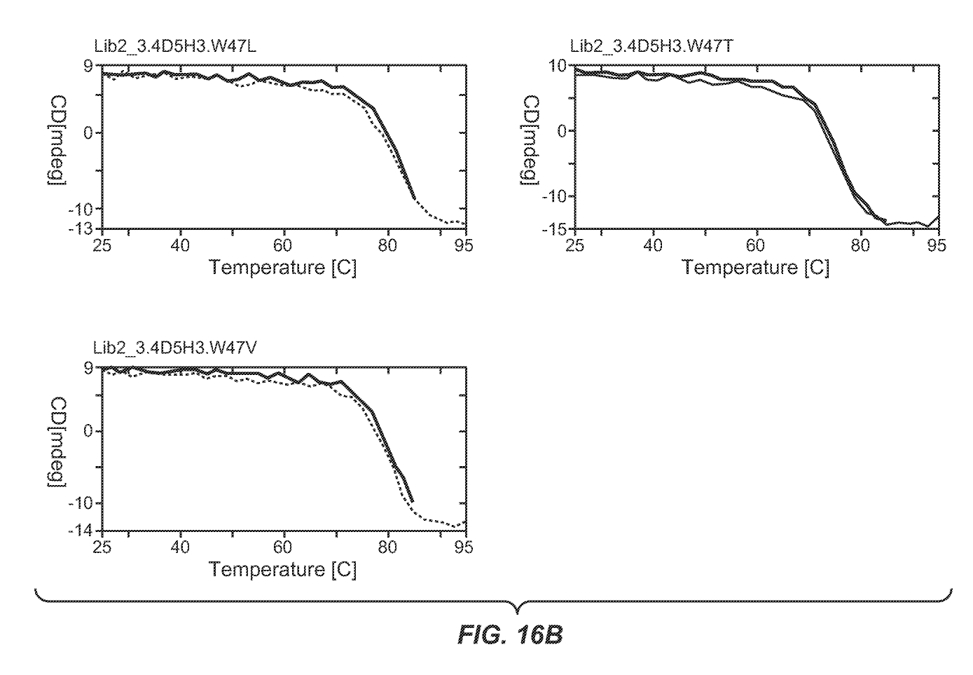

To investigate which other combinations of amino acids at positions 35 and 47 might support a stable VH domain scaffold, nine Lib2_{—}3 variants were constructed, expressed, purified, and characterized, as described above. These variants included G35S, R39D, R39E, W47L, W47V, W47A, W47T, W47E, and G35S/W47E. For all of the mutants, the wild-type 4D5 CDR-H3 was used, and the framework regions were modified at four positions (71A, 73T, 78A, and 93A) (see FIG. 1B). All variants were analyzed for proper protein folding by gel filtration and circular dichroism, as described above. The results are shown in Table 3 and FIGS. 15A-C and 16A-B.

All Lib2_{—}3 W47 mutants eluted from the gel filtration columns more rapidly than Lib2_{—}3.4D5H3 (30 minutes versus 40 minutes), and were approximately 90% monomer (FIGS. 15A-C). Each W47 mutant also displayed a Tₘ greater than 70° C. The Lib2_{—}3.4D5H3.W47L and Lib2_{—}3.4D5H3.W47V mutants displayed Tₘs close to 80° C., slightly greater than that of Lib2_{—}3.4D5H3. These results demonstrated that a tryptophan at position 47 was not necessary for maintaining the integrity of VH domain folding. Replacing the tryptophan with a smaller branched residue such as leucine or valine decreased aggregation of the VH domain while maintaining or even improving the thermal stability of the molecule.

TABLE 3
Properties of Lib2_3 Mutants

| | Yield | Calculated | Apparent | Aggregate | Tₘ | Reversible |
|---|---|---|---|---|---|---|
| Mutant | (mg/L) | MW (D) | MW (D) | % | (° C.) | folding? |
| Lib2_3 | 7 | 13043 | 14690 | ND | 75 | Yes |
| WT | | | | | | |
| 4D5H3 | | | | | | |
| G35S | 7 | 13073 | 16660 | 36 | ND* | ND |
| R39D | 5 | 13002 | 13260 | 12 | ND | ND |
| W47A | 2 | 12928 | 12450 | 14 | 75 | Yes |
| W47E | 3 | 12986 | 13240 | 8 | 75 | Yes |
| W47L | 6 | 12942 | 13590 | 9 | 80 | Yes |
| W47T | 6 | 12958 | 13430 | 10 | 75 | Yes |
| W47V | 7 | 12956 | 14210 | 12 | 80 | Yes |
| G35S/ | 5 | 13016 | 14360 | 8 | ND | ND |
| W47E | | | | | | |

*ND: not determined. Only those molecules having apparently improved characteristics by gel filtration analysis were further analyzed.

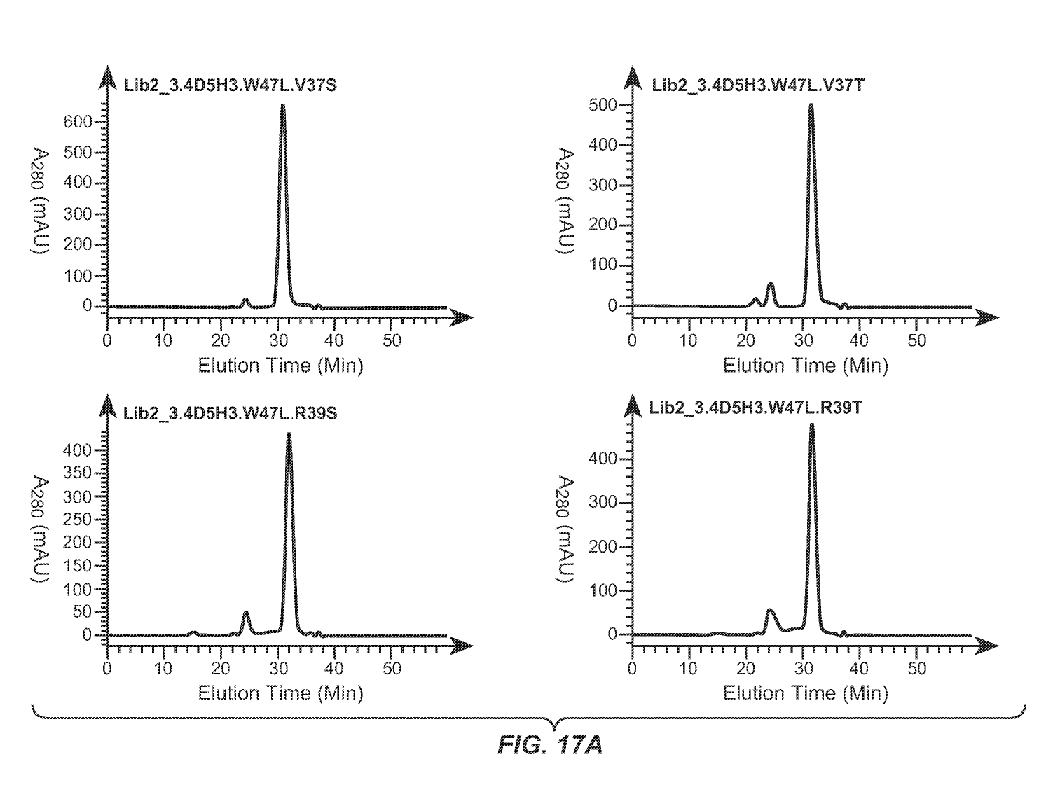

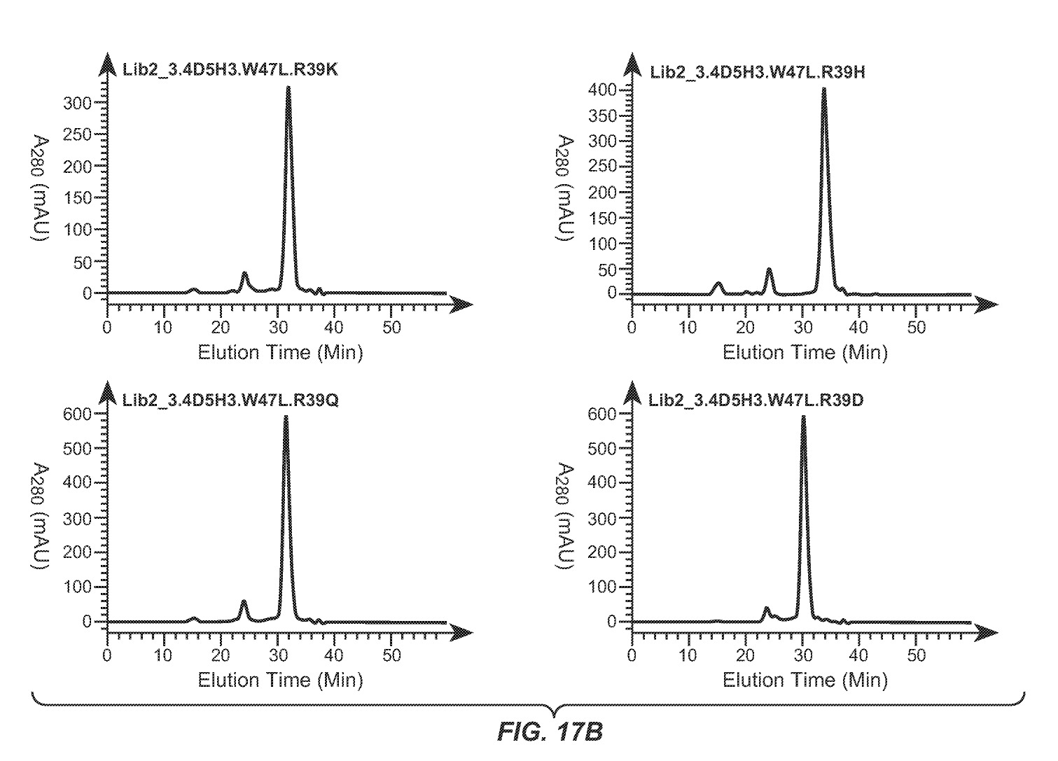

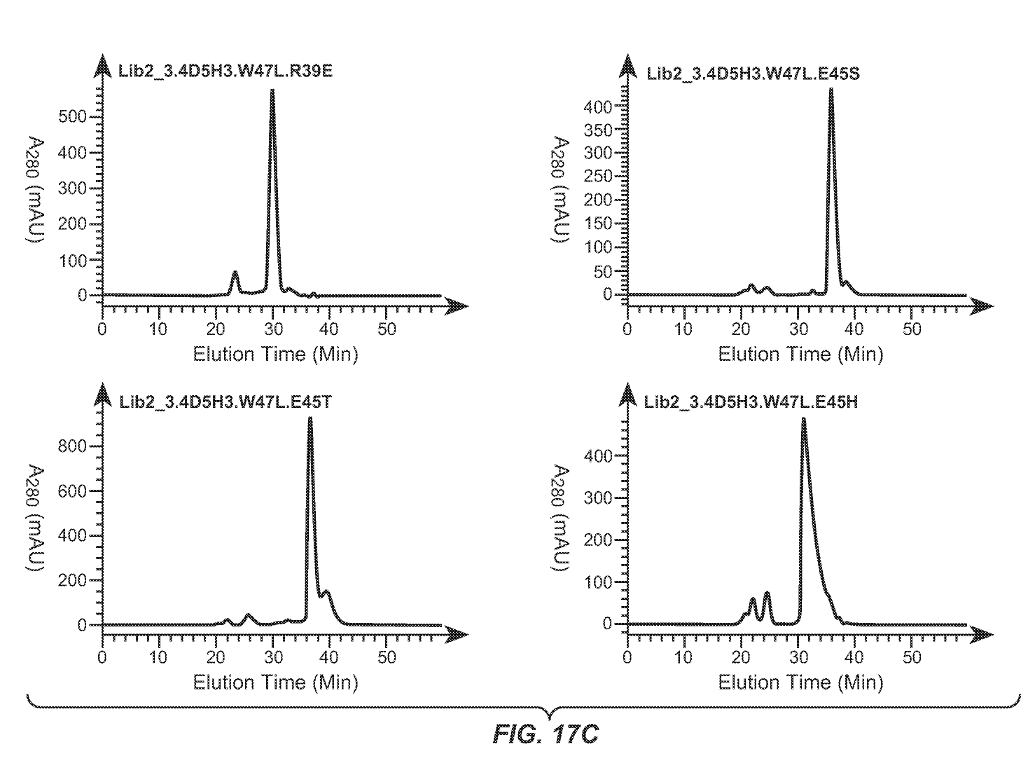

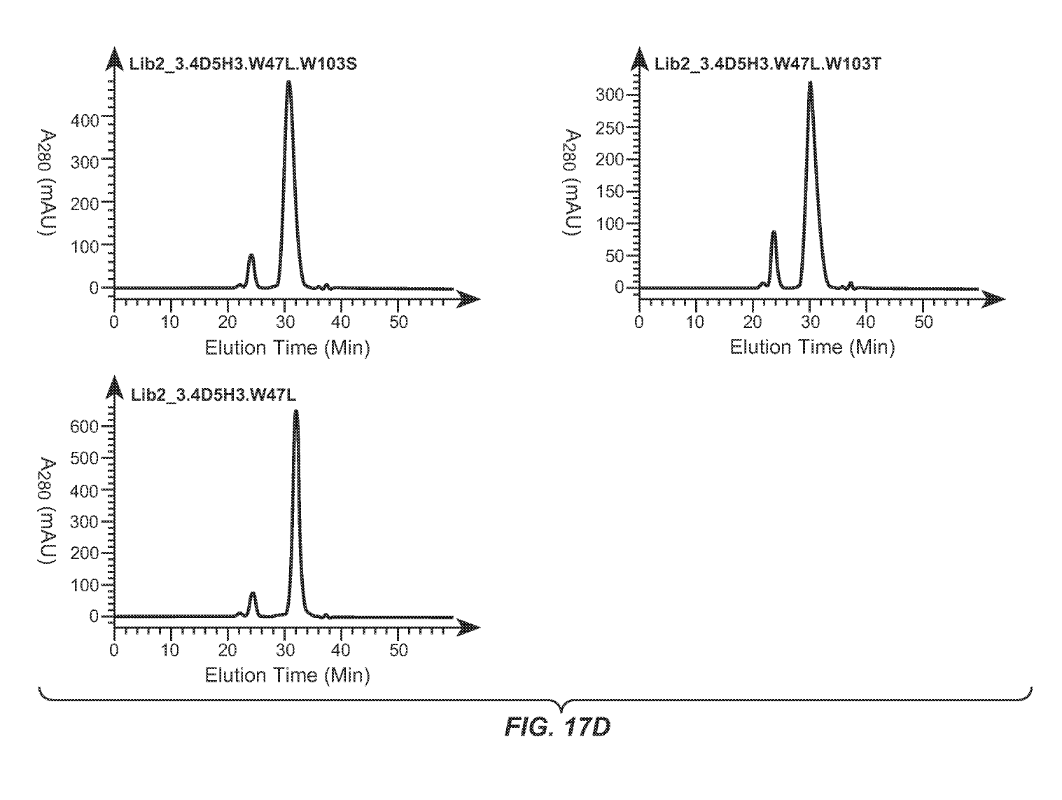

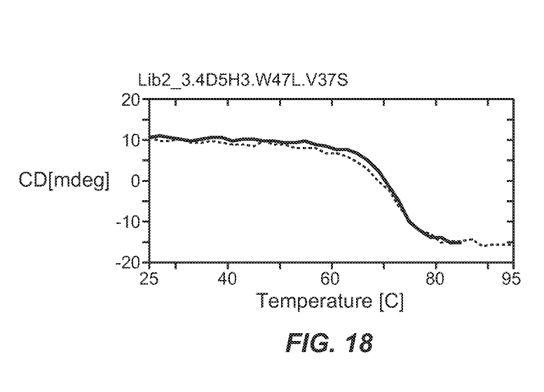

A further set of modified VH domains based on the Lib2_{—}3 framework was made to investigate whether a combination of the W47L mutation and another VL-interface residue mutation previously observed to have tolerated amino acid substitution (positions 37, 39, 45, or 103) might enhance the stability of the VH domain. Lib 2_{—}3.4D5H3.W47L and fourteen derived variants were constructed, expressed, purified, and characterized, as described above. These variants included W47L/V37S, W47L/V37T, W47L/R39S, W47L/R39T, W47L/R39K, W47L/R39H, W47L/R39Q, W47L/R39D, W47L/R39E, W47L/E45S, W47L/E45T, W47L/E45H, W47L/W103S, and W47L/W103T. For all of the mutants, the wild-type 4D5 CDR-H3 was used, and the framework regions were modified at four positions (71A, 73T, 78A, and 93A) (see FIG. 1B). All variants were analyzed for proper protein folding by gel filtration and circular dichroism, as described above. The results are shown in Table 4 and FIGS. 17A-D and 18.

Only one clone, Lib2_{—}3.4D5H3.W47L/V37S, showed an improved behavior in the gel filtration assay, eluting as approximately 97% monomeric at approximately 30 minutes. However, its yield was lower than that of earlier mutants (about 4 mg/L).

TABLE 4
Properties of Lib2_3 Double Mutants

| | | Calcu- | | | | |
|---|---|---|---|---|---|---|
| | | lated | | Aggre- | | Revers- |
| | Yield | MW | Apparent | gate | Tm | ible |
| Clone | (mg/L) | (D) | MW (D) | % | (° C.) | folding? |
| W47L | 7 | 12942 | 12800 | 10 | ND | ND |
| W47L/V37S | 3 | 12958 | 12910 | 3 | 73 | Yes |
| W47L/V37T | 5 | 12972 | 13340 | 11 | ND | ND |
| W47L/R39S | 8 | 12901 | 13110 | 10 | ND | ND |
| W47L/R39T | 6 | 12915 | 13410 | 16 | ND | ND |
| W47L/R39K | 9 | 12942 | 12640 | 10 | ND | ND |
| W47L/R39H | 8 | 12901 | 14680/15950 | 15 | ND | ND |
| W47L/R39Q | 7 | 12867 | 13450 | 10 | ND | ND |
| W47L/R39D | 5 | 12929 | 12910 | 12 | ND | ND |
| W47L/R39E | 2 | 12967 | 12780 | 12 | ND | ND |
| W47L/E45S | 8 | 12927 | 17400 | 17 | ND | ND |
| W47L/E45T | 6 | 12925 | 14620 | 30 | ND | ND |
| W47L/E45H | 7 | 12976 | 17730/18910 | 14 | ND | ND |
| W47L/ | 6 | 12871 | 12690 | 8 | ND | ND |
| W103S | | | | | | |
| W47L/ | 4 | 12885 | 12560 | 12 | ND | ND |
| W103T | | | | | | |

*ND: not determined. Only those molecules having apparently improved characteristics by gel filtration analysis were further analyzed.

### Example 5

### Contributions of CDR-H3 to VH Domain Stability in Certain Selectants

a. Alanine Scanning Analysis of CDR-H3 in Selectants from Library 1 and Library 2.

An ideal VH domain scaffold for constructing synthetic phage-displayed CH libraries should tolerate amino acid substitution in its CDRs to generate diversity while maintaining the overall stability of the domain through its fixed framework residues. The data from Library 1 showed a clear pattern of conservation in the region of the VH domain that interfaces with VL. However, no consensus sequences were observed in CDR-H3-containing loop of the VH domains, suggesting that that region was not involved in stabilizing the folding of most VH domains in the library. To confirm this analysis, an alanine shotgun-scanning combinatorial mutagenesis strategy was used to assess the contribution of each CDR-H3 loop residue to the folding of Lib1_{—}62 and the ten best-expressed domains from Library 2 (Lib2_{—}3, Lib2_{—}4, Lib2_{—}15, Lib2_{—}19, Lib2-48, Lib2_{—}56, Lib2_{—}61, Lib2_{—}87, Lib2_{—}89, and Lib2_{—}94).

Each of the amino acids in the CDR-H3 containing loop were alanine-scanned using phage-displayed libraries that preferentially allowed the side-chains of the randomized residues to vary between wild-type and alanine in equimolar proportions. Library construction was performed according to the procedure described in Example 1B. The stop-codon-containing oligonucleotides used were A22 (used for all clones except Lib2_{—}2, Lib2_{—}4 and Lib2_{—}94): ACT GCC GTC TAT TAT TGC TAA TAA TAA GGA ACA CTA GTC ACC GTC (SEQ ID NO: 232); oligonucleotide A24 (used for Lib2_{—}4):_ACT GCC GTC TAT AAA TGC TAA TAA TAA GGA ACA CTA GTC ACC GTC (SEQ ID NO: 233); and oligonucleotide B15 (used for Lib2_{—}94): ACT GCC GTC TAT TTT TGT TAA TAA TAA GGA ACA CTA GTC ACC GTC (SEQ ID NO: 234). The mutagenic oligonucleotides were as follows:

(SEQ ID NO: 235)

Oligo 5-1.
ACT GCC GTC TAT TAT TGC SST RCT KYT RCT RCT RMC
KCT RMA RMA GST KSG GST SMG GGA ACA CTA GTC ACC
GTC

(SEQ ID NO: 236)

Oligo 5-2.
ACT GCC GTC TAT AAC TGC RCT RCT SYG RCT KCT KCT
KYT RMA RYT KCT KSG GST GMT GGA ACA CTA GTC ACC
GTC

(SEQ ID NO: 237)

Oligo 5-3.
ACT GCC GTC TAT TAT TGC SST KCT SYG RCT RCT GMT
KCT RMA RCT GST SST GST SMG GGA ACA CTA GTC ACC
GTC

(SEQ ID NO: 238)

Oligo 5-4.
ACT GCC GTC TAT AAA TGC SST RCT KYT SCG RYG RMC
KCT RMA RMC GST KSG GST RMA GGA ACA CTA GTC ACC
GTC

(SEQ ID NO: 239)

Oligo 5-5.
ACT GCC GTC TAT TAT TGC SMG RCT KMT RCT RCT RMA
KCT RMA SST GST KCT GST SYG GGA ACA CTA GTC ACC
GTC

(SEQ ID NO: 240)

Oligo 5-6.
ACT GCC GTC TAT TAT TGC SST RCT KYT RMC RCT RMC
SYG GMA GST RCT KSG GST SCG GGA ACA CTA GTC ACC
GTC

(SEQ ID NO: 241)

Oligo 5-7.
ACT GCC GTC TAT TAT TGC KCT RCT KYT SMG GST RMC
RCT RMA RMA GYT KCT GST RMA GGA ACA CTA GTC ACC
GTC

(SEQ ID NO: 242)

Oligo 5-8.
ACT GCC GTC TAT TAT TGC GST RCT KYT KCT KCT RMC
KYT RMA RMA GST SST GST GMA GGA ACA CTA GTC ACC
GTC

(SEQ ID NO: 243)

Oligo 5-9.
ACT GCC GTC TAT TAT TGC RCT RCT KYT GST RCT SMG
KMT RMA RMA GST SST GST SYG GGA ACA CTA GTC ACC
GTC

(SEQ ID NO: 244)

Oligo 5-10.
ACT GCC GTC TAT TAT TGC GST RYG GYT KCT SCG RMA
GST SCG RYT KCT KSG GST SMG GGA ACA CTA GTC ACC
GTC

(SEQ ID NO: 245)

Oligo 5-11.
ACT GCC GTC TAT TAT TGC KCT RCT KMT RMC RCT RMA
SCG RMA GMA RCT SST GST RCT GGA ACA CTA GTC ACC
GTC

(SEQ ID NO: 246)

Oligo 5-12.
ACT GCC GTC TAT TTT TGC SST GST KYT KCT RCT GMT
KCT RMA SST GYT SST GST SST GGA ACA CTA GTC ACC
GTC

In each case, randomizations were performed using degenerate codons (where S corresponds to G or C; K corresponds to G or T; R corresponds to A or G; M corresponds to A or C; and Y corresponds to C or T), as indicated in the oligonucleotide sequences above.

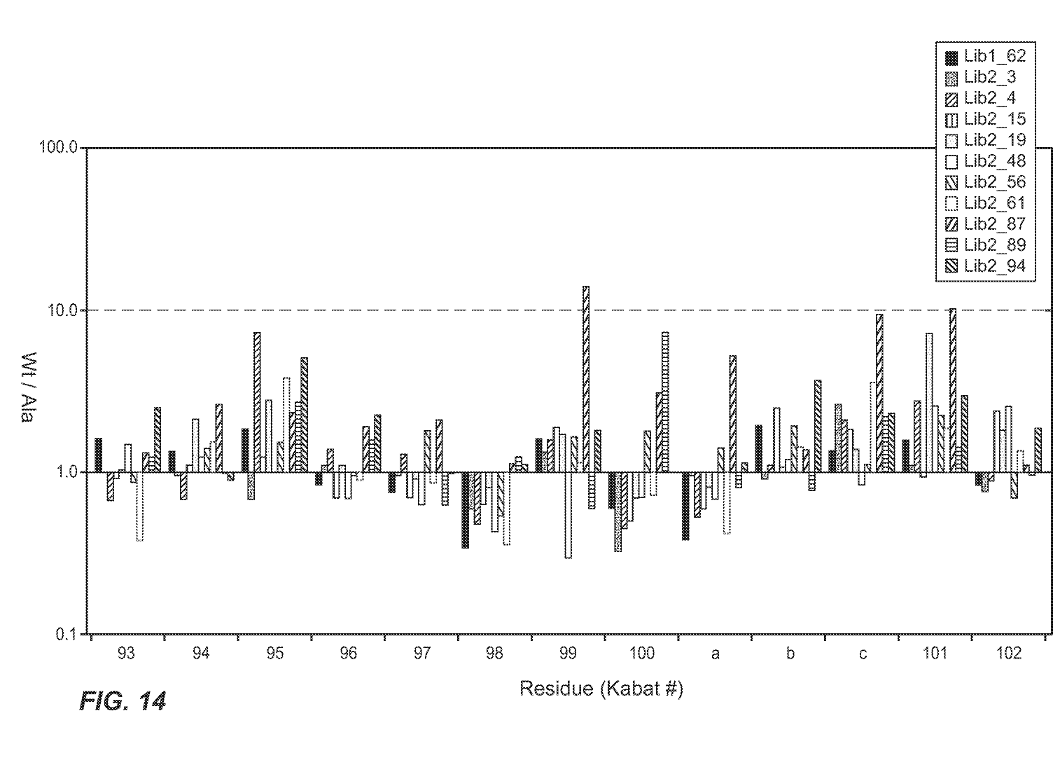

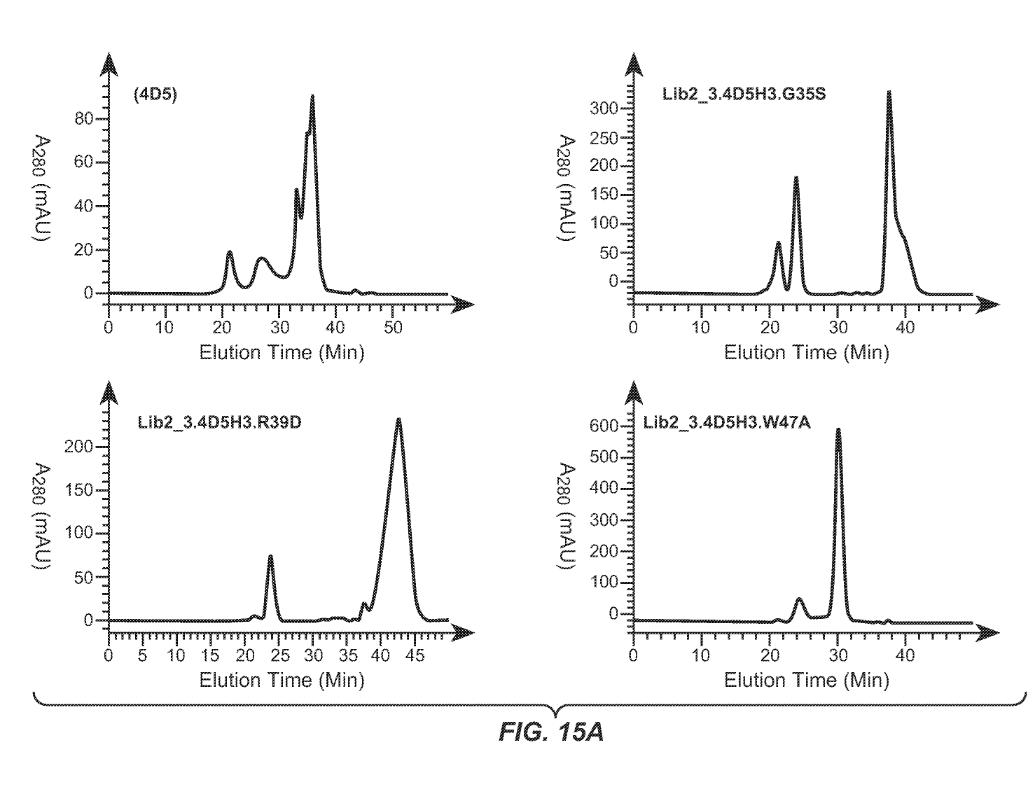

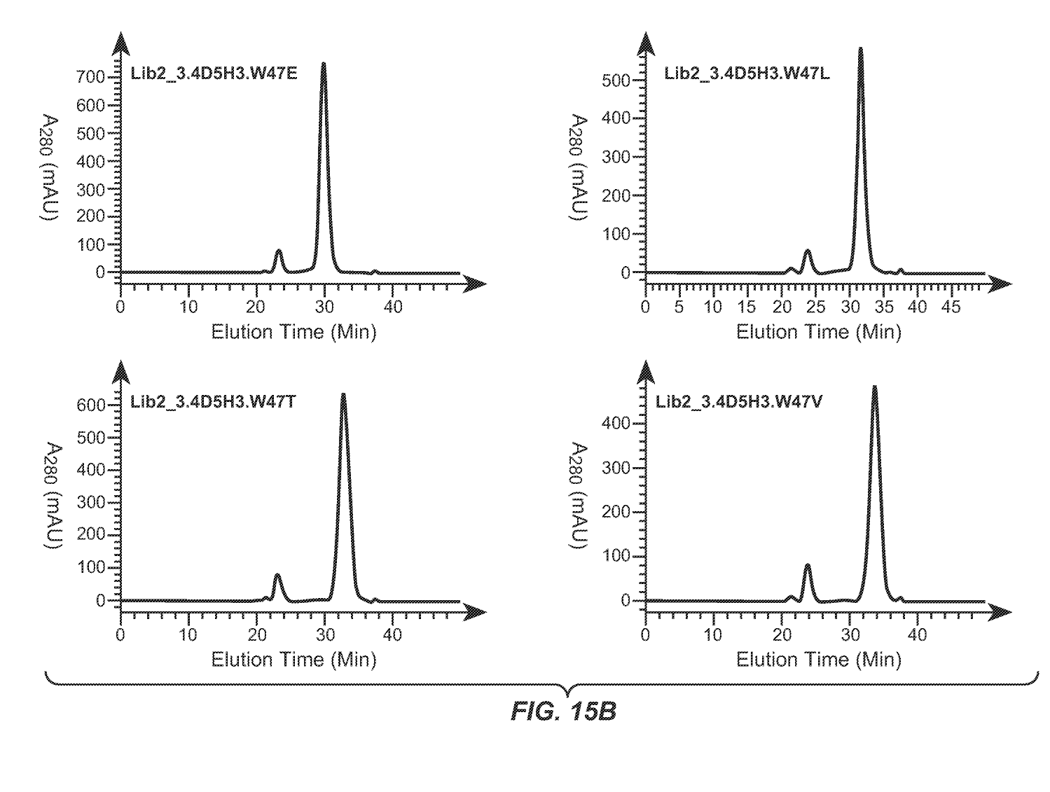

Library 5 was cycled through two rounds of affinity plate selection against Protein A to enrich for potentially highly stable variants. The methodology used was identical to that used for Library 1 (see Example 1C), but without an additional selection for binding to an anti-hexahistidine (SEQ ID NO: 274) antibody. After selection, 100 clones from each library were selected for sequencing, using the same methodology and primers as described in Example 1D. The wild-type/alanine ratio at each varied position was determined (FIG. 14), and those ratios were used to assess the contribution of each side-chain to the overall VH domain conformational stability.

CDR-H3 residues that are critical for the proper domain fold were not expected to tolerate alanine substitution, and therefore the wild-type residues should be strongly conserved at any such positions. Thus, residues presenting wild-type/alanine ratios greater than one represented residues that were important for VH domain stability. Residues presenting wild-type/alanine ratios less than one were tolerant to substitution. The CDR-H3 residues of the Lib1_{—}62 and Lib2_{—}3 VH domains had ratios close to 1 at all positions (see FIG. 14). Therefore, both clones were tolerant to alanine substitution in CDR-H3, and would serve as appropriate scaffolds for phage-displayed VH libraries in that they had highly stable domain folding but also a flexible CDR-H3 region to support diversity. On the contrary, clone Lib2_{—}87 exhibited several positions intolerant to alanine substitutions (e.g., positions 95, 99, 100a, 100c, and 101) (see FIG. 14). Consequently, no diversity could be introduced in its CDR-H3 without disrupting the overall domain stability.

b. Selected Mutational Analysis.

To confirm the alanine shotgun-scanning results, and to ensure that the CDR-H3 was not itself involved in Protein A binding, two Lib2_{—}3 mutants were constructed. In the first mutant, the Lib2_{—}3 CDR-H3 region was replaced by the wild-type 4D5 CDR-H3. In the second mutant, Protein A binding of Lib2_{—}3 was intentionally disrupted by replacing the threonine at position 57 with glutamate, resulting in a VH domain that should not bind normally to Protein A but which should still fold normally (Randen et al., Eur. J. Immunol. 23: 2682-86 (1993)). Both mutants were expressed and purified by Protein A chromatography as described in Example 1.

Lib2_{—}3.4D5H3, in which the Lib2_{—}3 CDR-H3 was replaced by the wild-type 4D5 CDR-H3 exhibited a high purification yield of about 11 mg/L, similar to, but lower than, the parent Lib2_{—}3 (up to 17 mg/L). Gel filtration/light scattering analysis showed that the variant was monomeric (Table 2). The Lib2_{—}3.4D5H3 Tₘ was close to 80° C., and its melting curve was fully reversible (Table 2). The results demonstrate that CDR-H3 was not significantly involved in the structural stability of the Lib2_{—}3 VH domain.

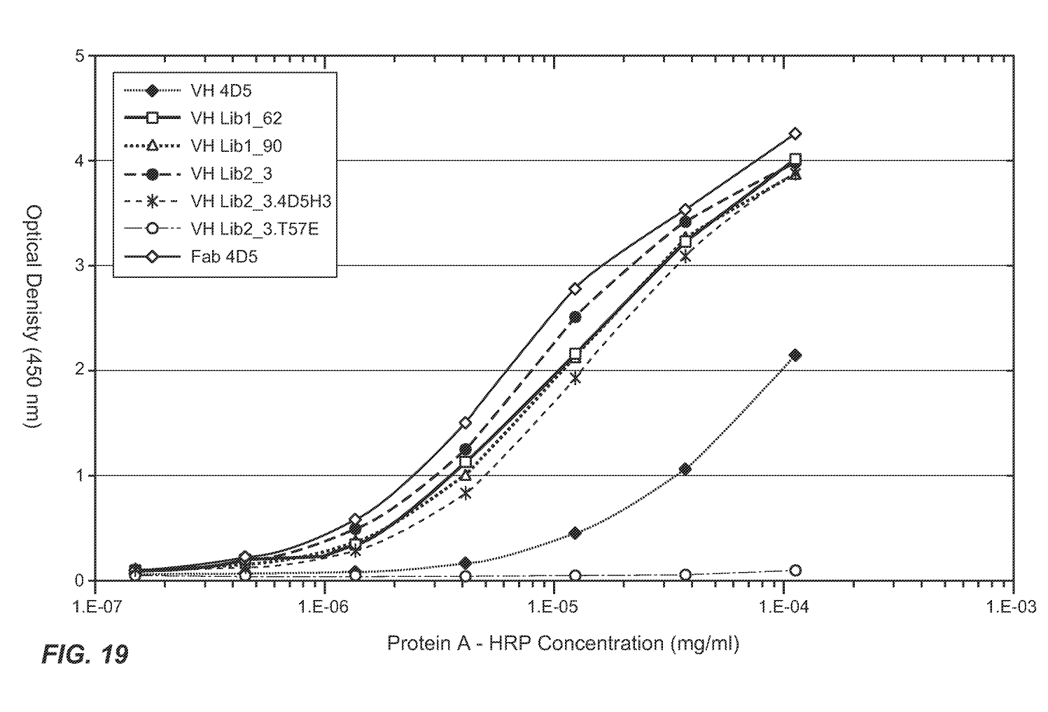

Lib2_{—}3.T57E, in which the threonine at position 57 was altered to glutamate, exhibited a low purification yield (around 2.5 mg/L) in the Protein A chromatography assay. A Protein A ELISA assay confirmed that binding to Protein A was effectively disrupted in that mutant VH domain (FIG. 19). Lib2_{—}3.T57E was monomeric in the gel filtration/light scattering assay, and its Tₘ and melting curve were similar to that of Lib2_{—}3 (Table 2), indicating that the Lib2_{—}3.T57E VH domain was correctly folded. Thus, the Lib2-3 CDR-H3 domain was not significantly involved in Protein A binding.

### Example 6

### Crystallographic Analysis of VH-B1a

Further experiments were undertaken to better understand the molecular basis for the high stability of the Lib2_{—}3.4D5H3 VH domain mutant. A version of Lib2_{—}3.4D5H3 was constructed lacking the histidine tag and having a modified linker region between the VH domain and the phage coat protein 3 open reading frames. The histidine tag tail was first removed and the linker modified by Kunkel mutagenesis using oligonucleotide E1 (GTC ACC GTC TCC TCG GAC AAA ACT CAC ACA TGC GGC CGG CCC TCT GGT TCC GGT GAT TTT (SEQ ID NO: 251)), using the procedures described above. An amber stop was introduced using Kunkel mutagenesis with oligonucleotide G1 (CTA GTC ACC GTC TCC TCG TAG GAC AAA ACT CAC ACA TGC (SEQ ID NO: 252)), following the procedures described above. The resulting molecule was named VH-B1a.

A crystallographic analysis of the VH-B1a protein was performed. Large scale preparation of VH-B1a domain was performed as described in Example 1 (D)(1) above. Following Protein A purification, 10 mg of the domain were loaded on a Superdex™ HiLoad™ 16/60 column (Amersham Bioscience) with 20 mM Tris pH 7.5, 0.5 M NaCl as mobile phase at a flow rate of 0.5 ml/min. The VH domain was then concentrated to 10 mg/ml. Sitting-drop experiments were performed by using the vapor-diffusion method using 2 μl drops consisting of a 1:1 ratio of protein solution and reservoir solution (1. 1 M sodium malonate pH 7.0, 0.1 M Hepes pH 7.0, 0.5% v/v Jeffamine ED-2001 pH7.0). Crystals grew after 1 week at 19° C. The resulting crystals were visibly not single and were broken down into smaller entities. Resulting crystals were directly flash frozen in liquid nitrogen. A data set was collected at the Stanford Synchrotron Radiation Laboratory (Stanford University).

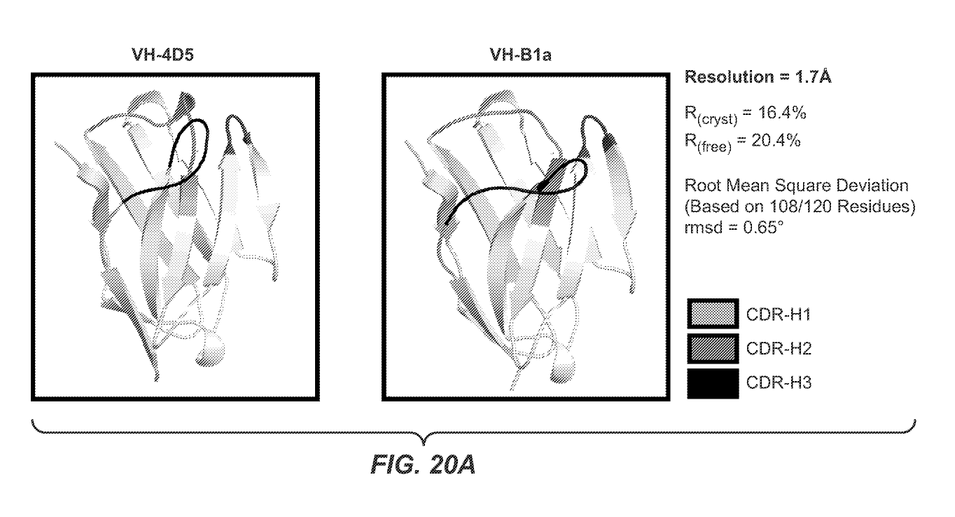

The data were processed by using the programs Denzo and Scalepack (Z. Otwinowski and W. Minor, Methods in Enzymology, Volume 276: Macromolecular Crystallography, part A, p. 307-326, 1997,C. W. Carter, Jr. & R. M. Sweet, Eds., Academic Press (New York)]. The structures were solved by molecular replacement using the program Phaser (McCoy et al. Acta Crystallogr D Biol Crystallogr, 2005 April; 61(Pt 4):458-64) and the coordinates of a solved Herceptin molecule (PDB entry 1N8Z). The structure was refined using the program REFMAC (Murshudov et al. Acta Crystallogr D Biol Crystallogr, 1997 May 1; 53(Pt 3):240-55). The model was manually adjusted using the program Coot (Emsley et al. Acta Crystallogr D Biol Crystallogr, 2004 December; 60(Pt 12 Pt 1):2126-32). VH-B1a crystallized in space group P1 with unit cell dimension of a=50.9 Å, b=54.1 Å, c=54.2 Å, α=110°, β=95.6° and γ=119°. The structure consists of 4 molecules per asymmetric unit. The resolution of the crystal structure was 1.7 Å. R_{(cryst)} was 16.4% and R_{(free)} was 20.4%, with a root mean square deviation (calculated with framework Calpha atoms of the 1N8Z VH domain for molecular replacement) of 0.650 (based on 108 of 120 residues). The structure is shown in FIG. 20A, right panel.

In contrast with the 4D5 VH domain structure (FIG. 20A, left panel), the CDRH3 loop region in VH-B1a (FIG. 20A, right panel) was shifted to be in closer proximity to the bulk of the molecule. The remainder of the VH-B1a structure was similar to that of the Herceptin VH domain (FIG. 20A) (Cho et al. Nature. 2003 Feb. 13; 421(6924):756-60), as indicated by the small rmsd of 0.63 Å.

A previous study using a modified VH domain had shown that the sidechain of a tryptophan at position 47 interacted with the cavity formed by replacement of a serine at position 35 with a glycine (Jespers et al., J. Mol. Biol. 337: 893-903 (2004)) (FIG. 20B, upper right panel), resulting in a more stable VH domain. A closer examination of the VH-B1a structure surprisingly revealed a reorientation of the side chains of Trp95 and Trp 103 from their positions in the Herceptin VH domain structure. Both of those tyrosine sidechains were flipped into a cavity formed following the replacement of His35 by a glycine (FIG. 20B, compare bottom right panel to bottom left panel). The sidechain of Trp47, however, did not notably change orientation between the 4D5 VH domain structure and the VH-B1a structure (FIG. 20B, compare bottom left panel to bottom right panel), unlike the structure of VH-Hel4 (FIG. 20B, compare upper and lower right panels).

One possible explanation for these data is that the Lib2_{—}3.4D5H3 VH domain mutant has enhanced stability relative to the wild-type 4D5 VH domain because the sidechains of Trp95 and Trp103 fit into the cavity created by the presence of a glycine at position 35. This interaction may limit the flexibility of the CDRH3 loop, and may lead to stabilization of the structure by, e.g., minimizing unfolding or preventing aberrant folding that would normally lead to aggregation and/or degradation. The above data show that while the sidechain of Trp47 may interact with the Gly35 cavity in certain circumstances (Jespers, J. Mol. Biol. 337: 893-903 (2004)), other proximal tryptophans may preferentially interact with the Gly35 cavity even in the presence of Trp47.

### Example 7

### Further Analysis of the B1a Variant

a. Oligomeric State Equilibrium Analysis

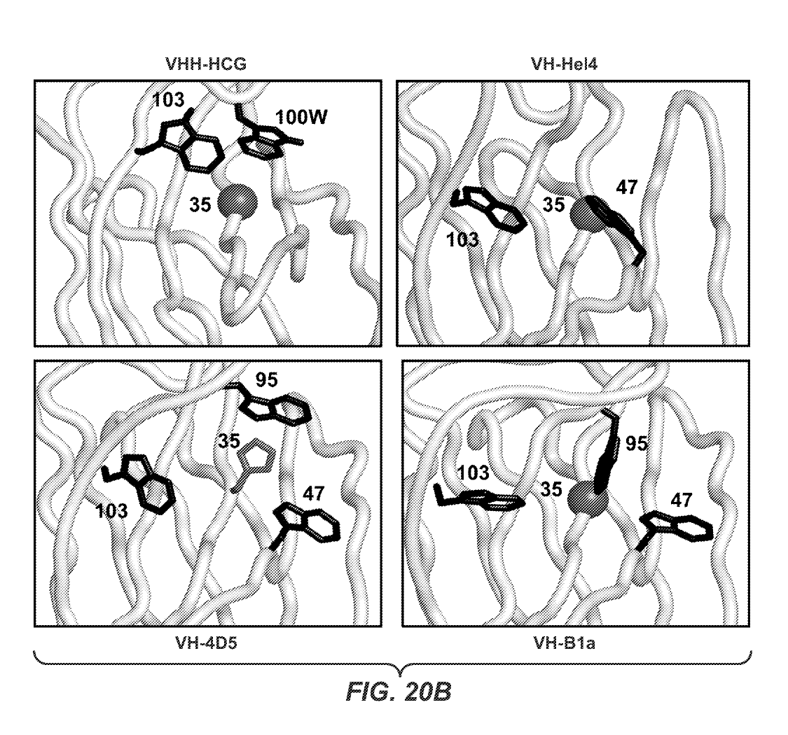

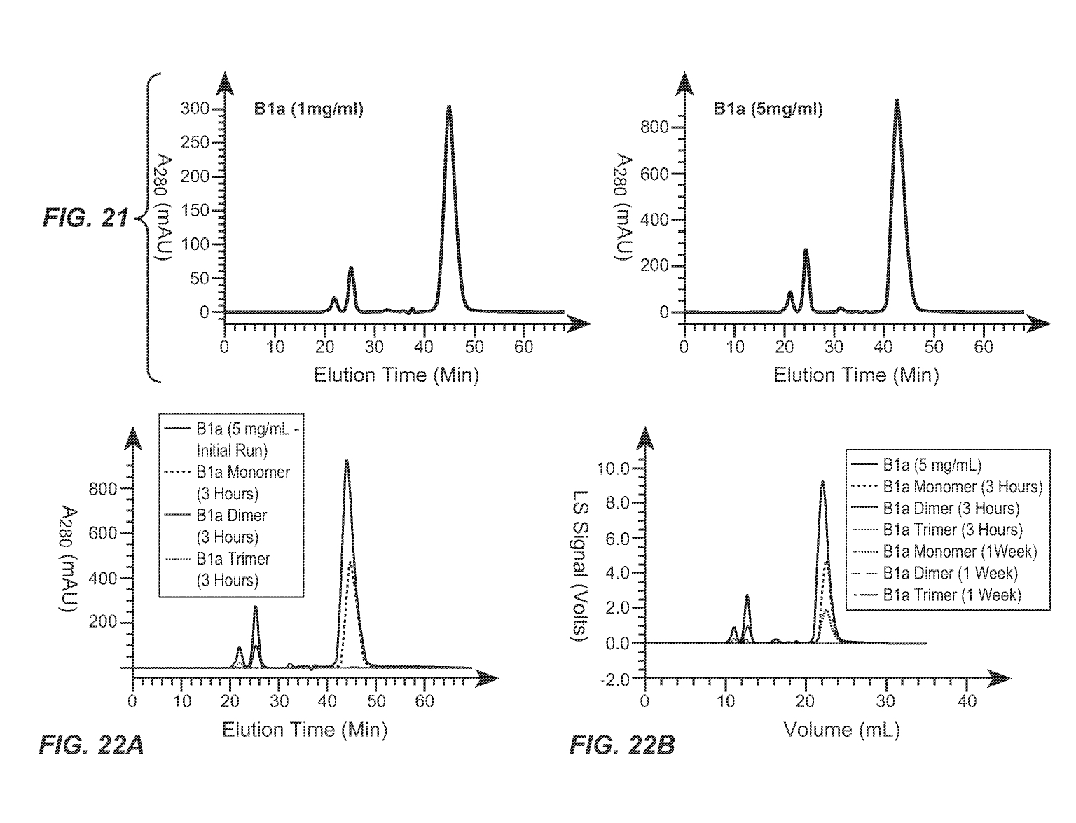

The oligomeric state of the B1a variant was assessed by gel filtration, using the light scattering procedure described in Example 1 (D)(2), above. As shown in FIG. 21, the B1a variant eluted as a series of three distinct peaks: largely monomer, but with some dimer and trimer peaks also visible. Generalized aggregation was not observed in the B1a variant, unlike wild-type VH domain, LibA2_{—}45, LibA2_{—}66, and LibA3_{—}87. Further experiments were performed to ascertain whether a dynamic equilibrium existed between the monomer, dimer, and trimer forms of the B1a variant, or whether each form was a stable entity.

The B1a variant was expressed in E. coli and purified using Protein A as described above (see Example 1D(1)). Two different concentrations of the purified B1a protein (1 mg/mL and 5 mg/mL) were then passed through a sizing column as described above (see Example 1D(1)). Identical elution profiles and similar oligomeric state ratios were obtained for both concentrations (see Table 5), demonstrating that the observed B1a protein multimerization was concentration-independent at least up through 5 mg/mL. The peaks corresponding to the monomer, dimer, and trimer forms from the 5 mg/mL sample run were collected individually and re-injected on the same gel filtration column approximately 3 hours after the initial run. As shown in FIG. 22A, the ratios of monomer, dimer, and trimer remained constant in this second sizing column run relative to the ratios observed in the first sizing column run. The monomer, dimer, and trimer fractions were stored at 4° C. for one week, and then were run again on the sizing column. As shown in FIG. 22B, the results were similar to those observed in the initial run, indicating that the monomer, dimer, and trimers of B1a are fairly stable.

TABLE 5
Recovery Times and Yields of Monomer, Dimer, Trimer Forms of B1a

| | | |
|---|---|---|
| Multimer State | Time (min) | Area % |

B1a (1 mg/mL)

| | | |
|---|---|---|
| Trimer | 22 | 3 |
| Dimer | 25 | 9 |
| Monomer | 45 | 88 |

B1a (5 mg/mL)

| | | |
|---|---|---|
| Trimer | 22 | 4 |
| Dimer | 25 | 11 |
| Monomer | 44 | 85 |

Reinjected monomer from B1a (3 hours)

| | | |
|---|---|---|
| Trimer | 22 | 0 |
| Dimer | 25 | 0 |
| Other | 40 | 1 |
| Monomer | 45 | 99 |

Reinjected monomer from B1a (1 week)

| | | |
|---|---|---|
| Trimer | 20 | 1 |
| Dimer | 24 | 1 |
| Other | 43 | 2 |
| Monomer | 45 | 96 |

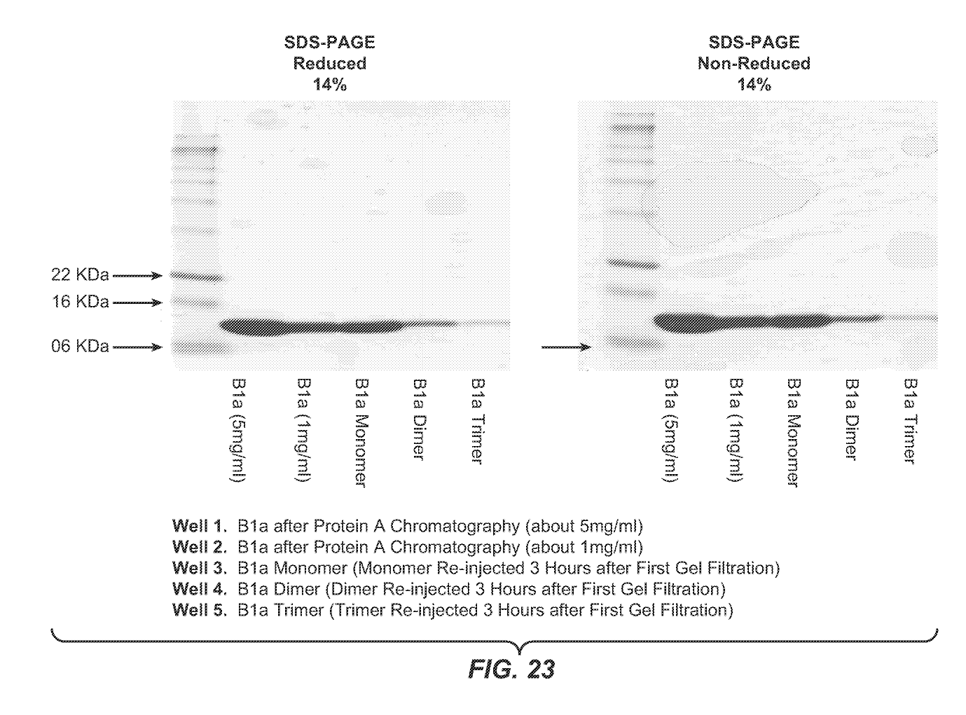

To further characterize the stable B1a protein dimer and trimer formation, samples were analyzed on both denaturing and non-denaturing SDS-polyacrylamide gels (see FIG. 23). In each gel, the first and second lanes represent the protein pool after Protein A purification at 5 mg/mL or 1 mg/mL, and the other three lanes in each gel show the re-injected monomer, dimer, and trimer forms of the protein. Because both gels showed all samples migrating at approximately 13 kDa, the size of the monomeric form, it was apparent that the formation of the dimer and trimer forms was not dependent on disulfide bond formation (compare left and right panels in FIG. 23).

Thus, the monomer, dimer, and trimer forms of the B1a protein were separable, stable, and apparently not due to disulfide bond formation. A possible explanation for multimerization of these proteins is that they may result from a strand swap mechanism, as has been observed previously in certain camelid VH domains (Spinelli et al., 2004, FEBS Lett. 564(1-2): 35-40).

b. Construction of VH-B1a Variants with Point Mutations in the Former Light Chain Interface

Having found that the B1a protein was largely free from aggregation and stable in solution, a series of B1a mutants containing point mutations in the former light chain interface were constructed in order to determine the individual contribution of each residue in VH domain B1a that differed from the wild-type 4D5 sequence. A series of mutant B1a VH domains were prepared in which each of the substituted amino acids was mutated back to the wild-type counterpart in three different position 47 backgrounds: tryptophan, leucine, or threonine. Twelve mutant B1a VH domains were constructed using Kunkel mutagenesis as described above: B1a(G35H/W47); B1a(G35H/W47L); B1a(G35H/W47T); B1a(Q39R/W47); B1a(Q39R/W47L); B1a(Q39R/W47T); B1a(E45L/W47); B1a(E45L/W47L); B1a(E45L/W47T); B1a(S50R/W47); B1a(S50R/W47L); and B1a(S50R/W47T). The oligonucleotides used in the mutagenesis were as follows:

(SEQ ID NO: 253)

G34 (G35H mutation)
ATT AAA GAC ACC TAT ATA CAC TGG GTC CGT CGG GCC

(SEQ ID NO: 254)

G35 (L47W mutation)
GGT AAG GGC GAG GAA TGG GTT GCA AGT ATT TAT CCT

(SEQ ID NO: 255)

G36 (L47T mutation)
GGT AAG GGC GAG GAA ACC GTT GCA AGT ATT TAT CCT

(SEQ ID NO: 256)

G37 (R39Q/L47W mutations)
TAT ATA GGA TGG GTC CGT CAG GCC CCG GGT AAG GGC
GAG GAA TGG GTT GCA AGT ATT TAT CCT

(SEQ ID NO: 257)

G38 (R39Q mutation)
TAT ATA GGA TGG GTC CGT CAG GCC CCG GGT AAG GGC
GAG

(SEQ ID NO: 258)

G39 (R39Q/L47T mutations)
TAT ATA GGA TGG GTC CGT CAG GCC CCG GGT AAG GGC
GAG GAA ACC GTT GCA AGT ATT TAT CCT

(SEQ ID NO: 259)

G40 (E45L/L47W mutations)
CGG GCC CCG GGT AAG GGC CTG GAA TGG GTT GCA AGT
ATT TAT CCT

(SEQ ID NO: 260)

G41 (E45L mutation)
CGG GCC CCG GGT AAG GGC CTG GAA CTG GTT GCA AGT
ATT

(SEQ ID NO: 261)

G42 (E45L/L47T mutations)
CGG GCC CCG GGT AAG GGC CTG GAA ACC GTT GCA AGT
ATT TAT CCT

(SEQ ID NO: 262)

G43 (L47W/S50R mutations)
CCG GGT AAG GGC GAG GAA TGG GTT GCA CGT ATT TAT
CCT ACG AAT GGT

(SEQ ID NO: 263)

G44 (S50R mutation)
GGC GAG GAA CTG GTT GCA CGT ATT TAT CCT ACG AAT
GGT

(SEQ ID NO: 264)

G45 (L47T/S50R mutations)
CCG GGT AAG GGC GAG GAA ACC GTT GCA CGT ATT TAT
CCT ACG AAT GGT

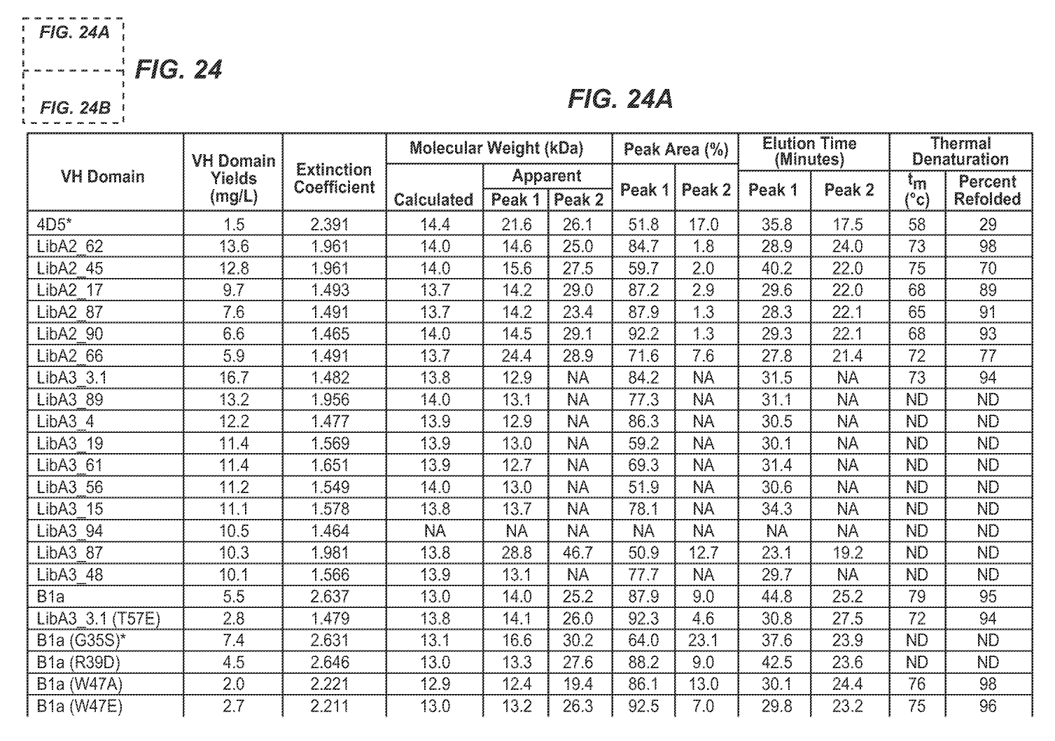

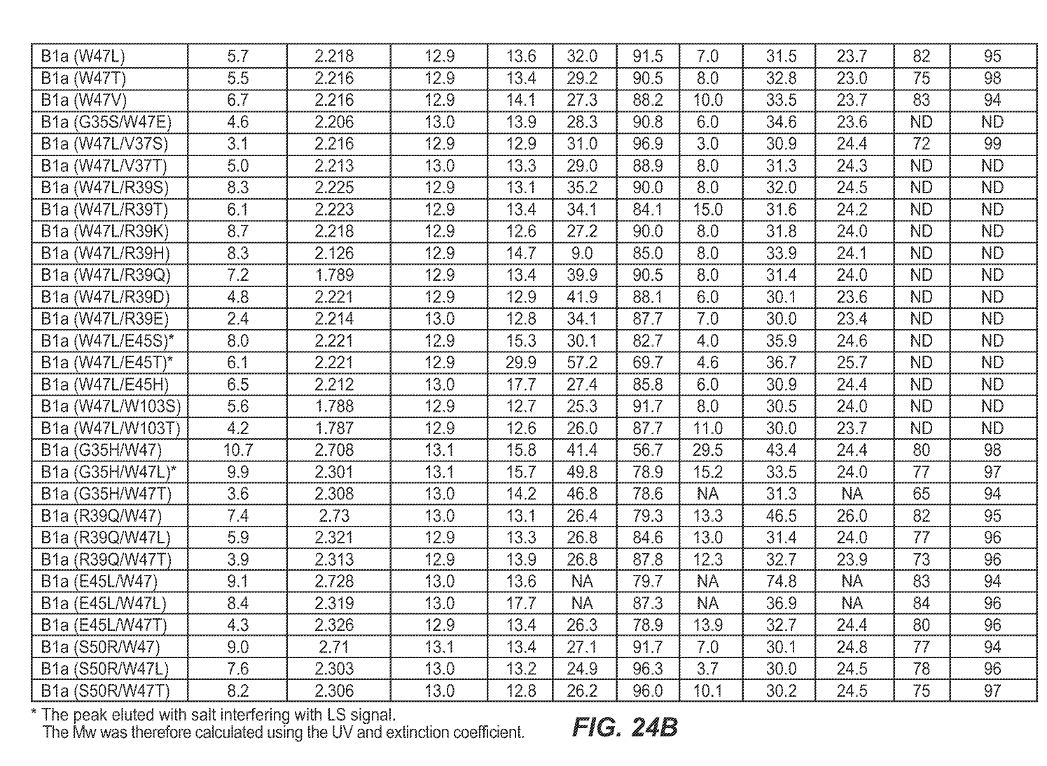

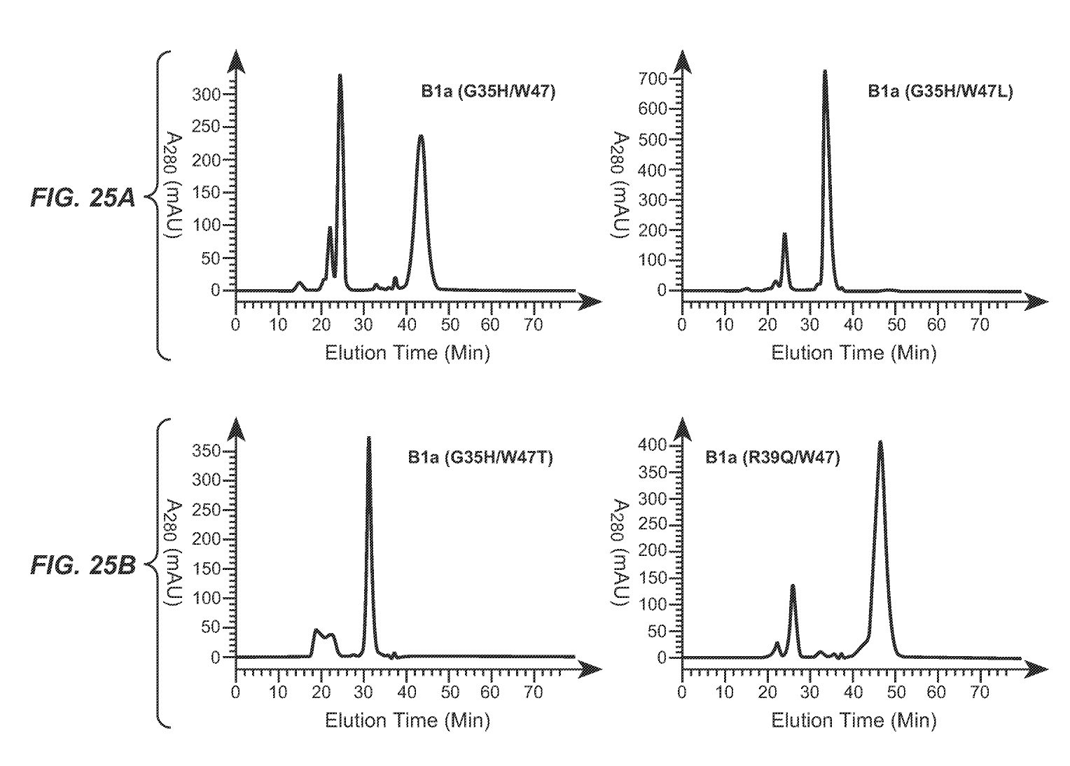

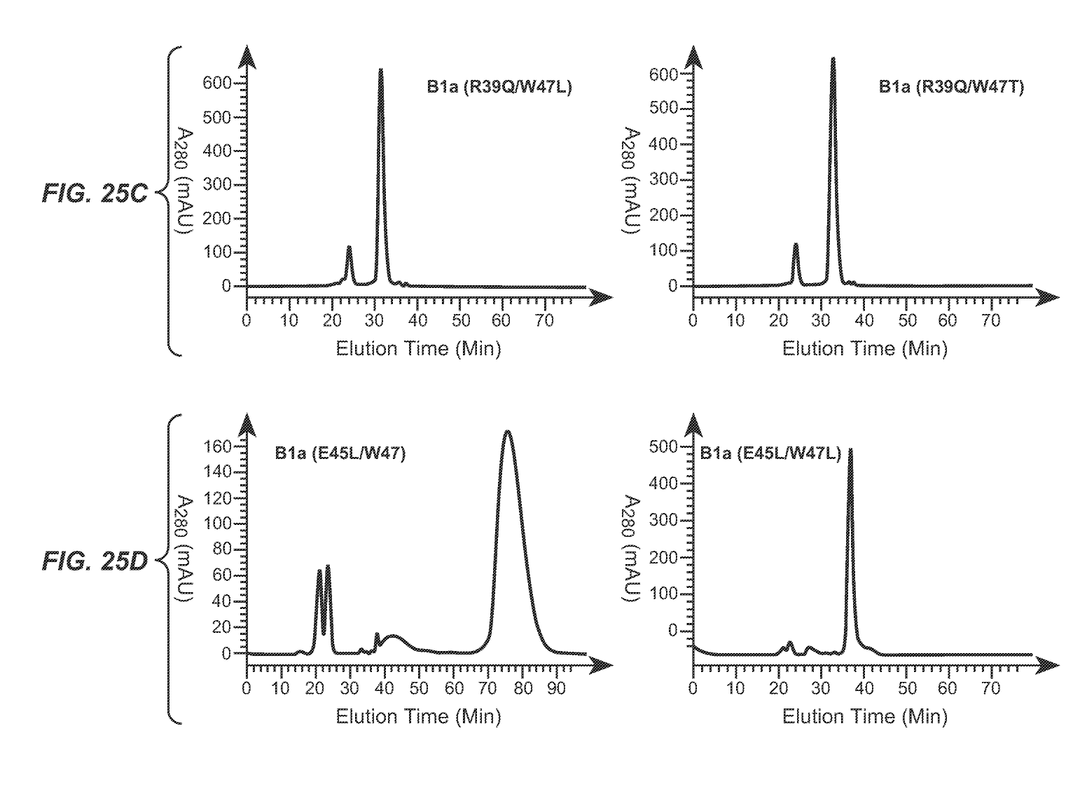

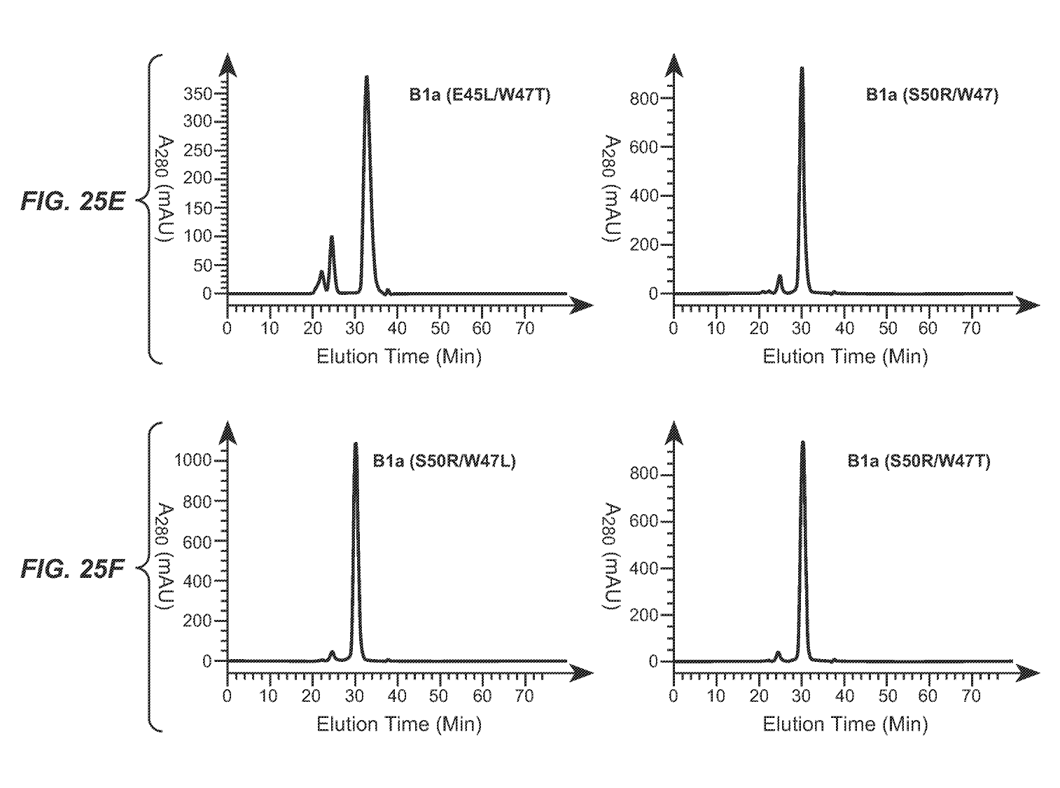

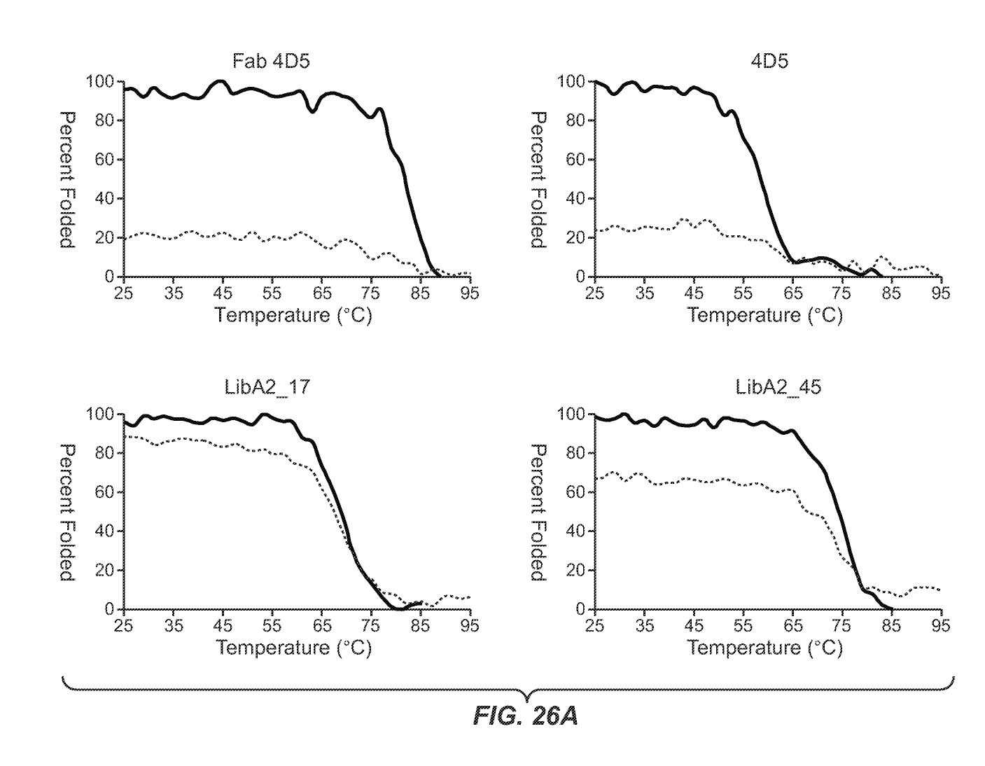

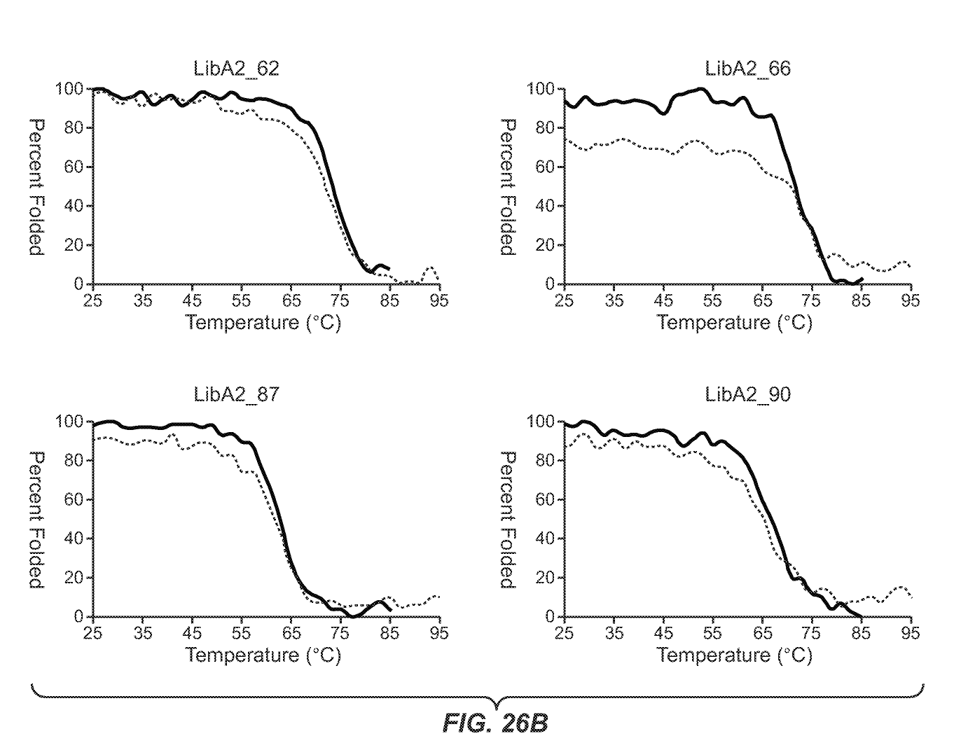

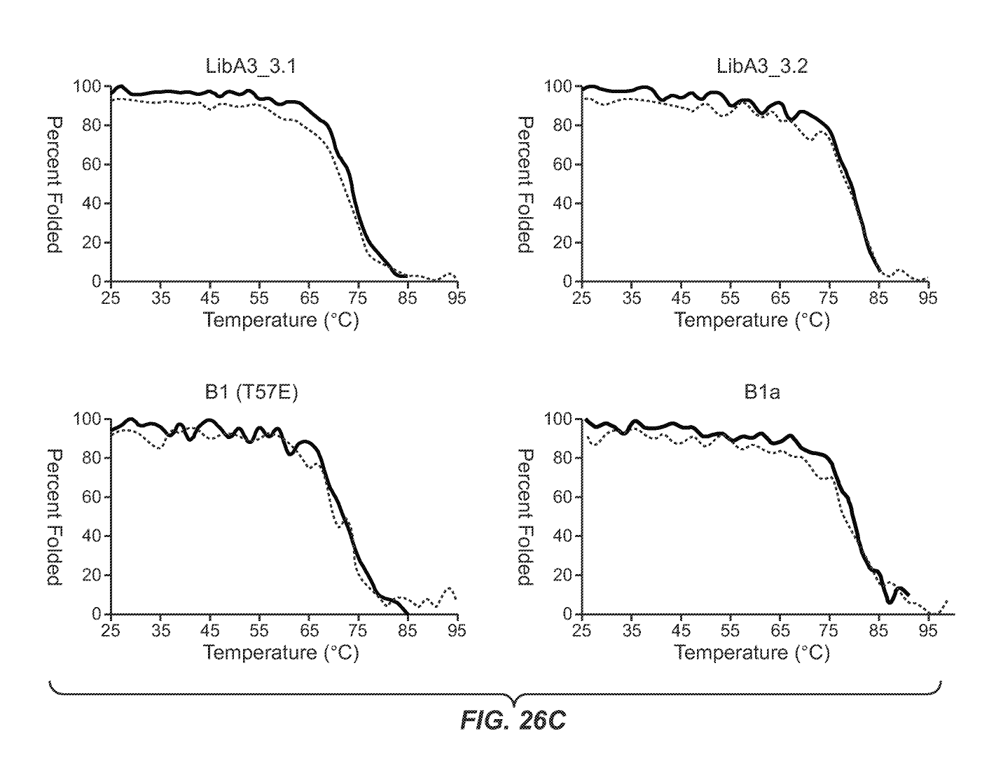

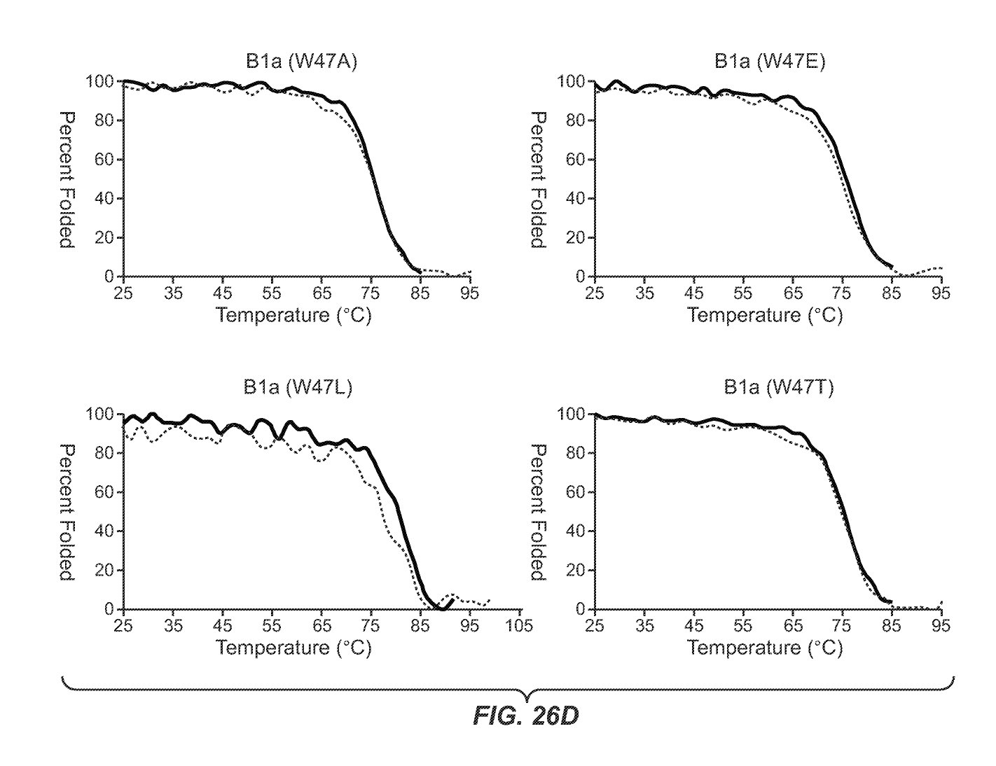

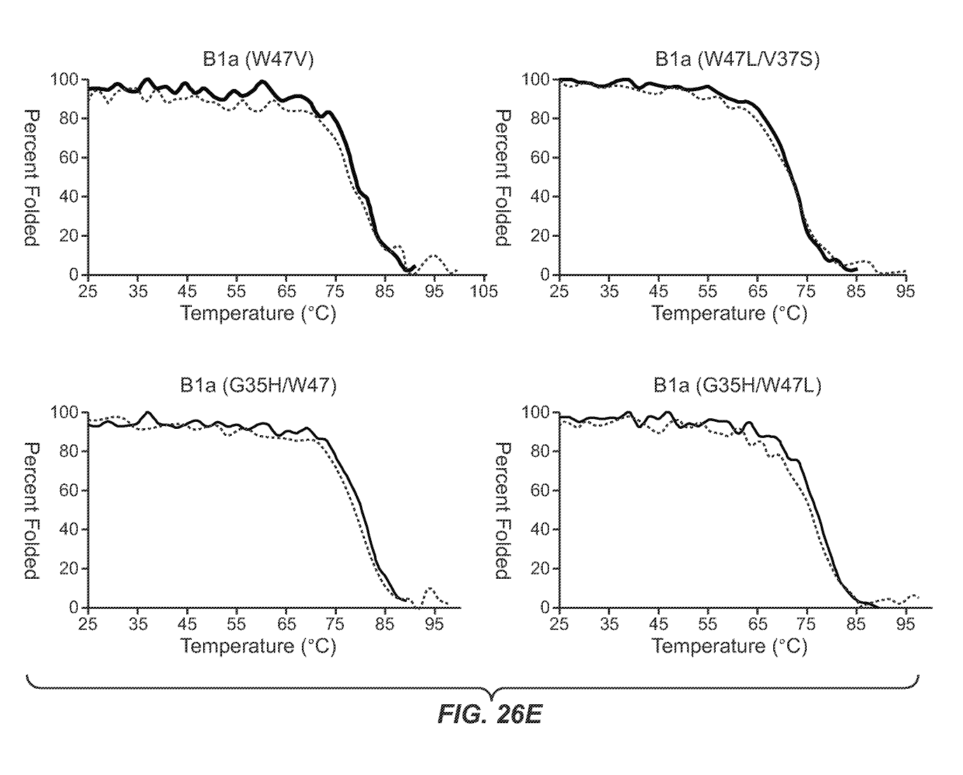

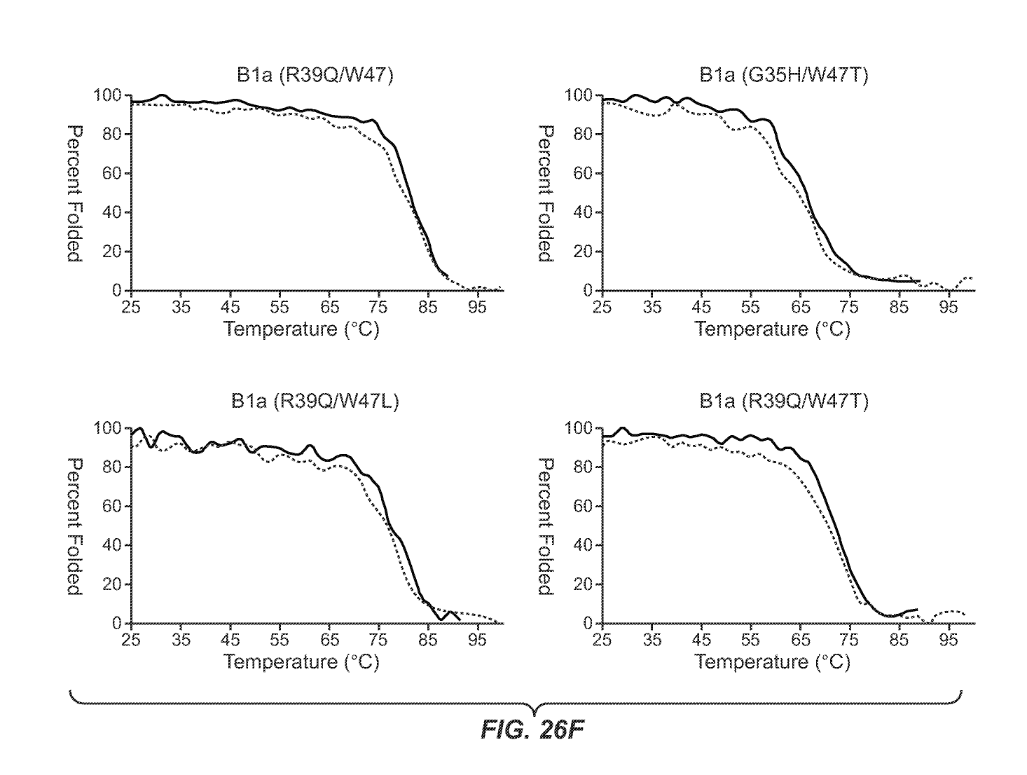

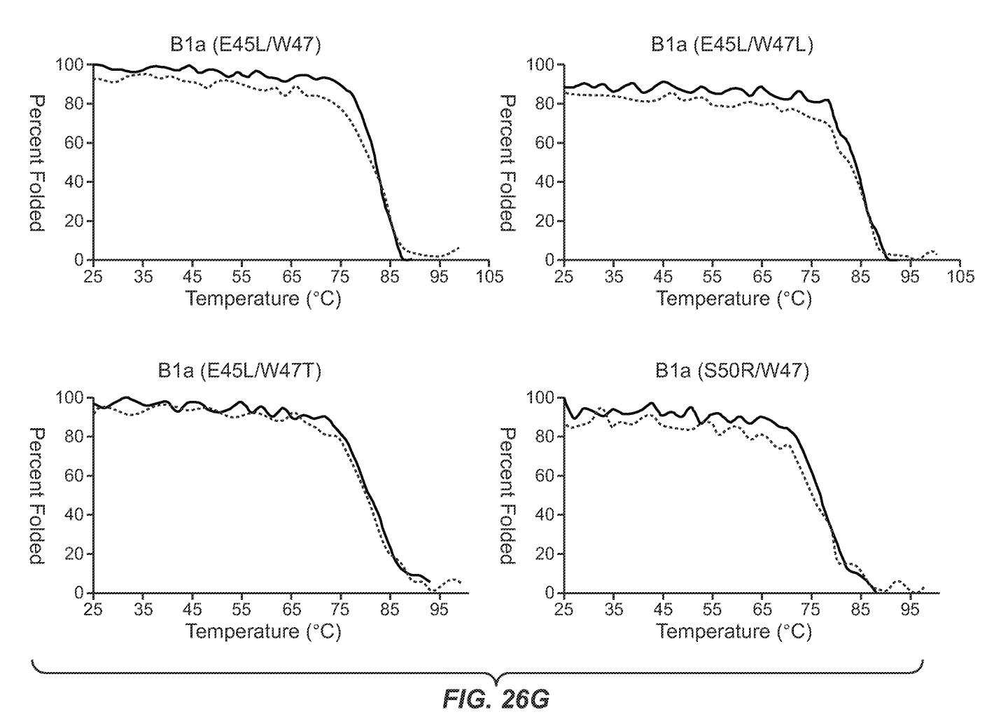

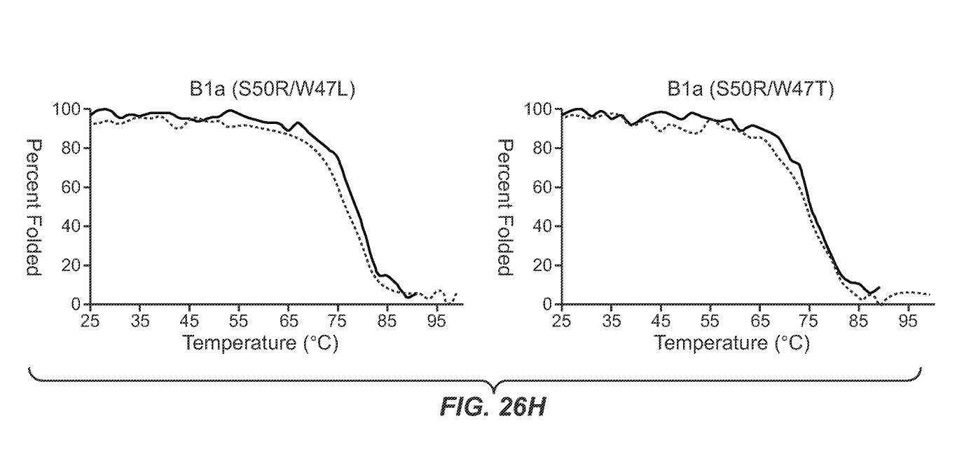

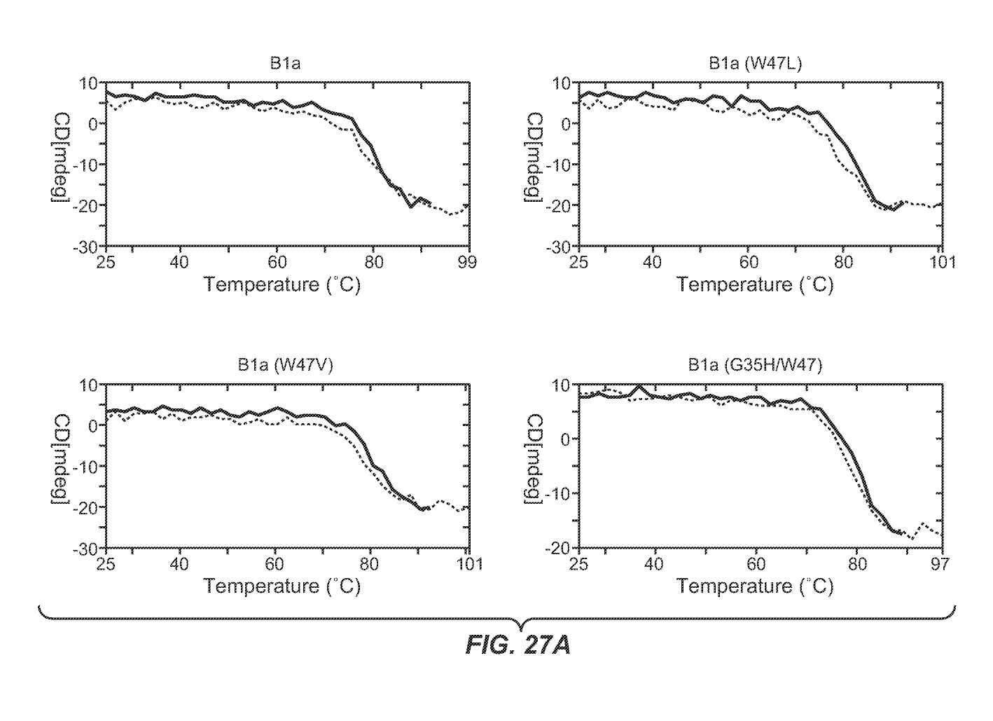

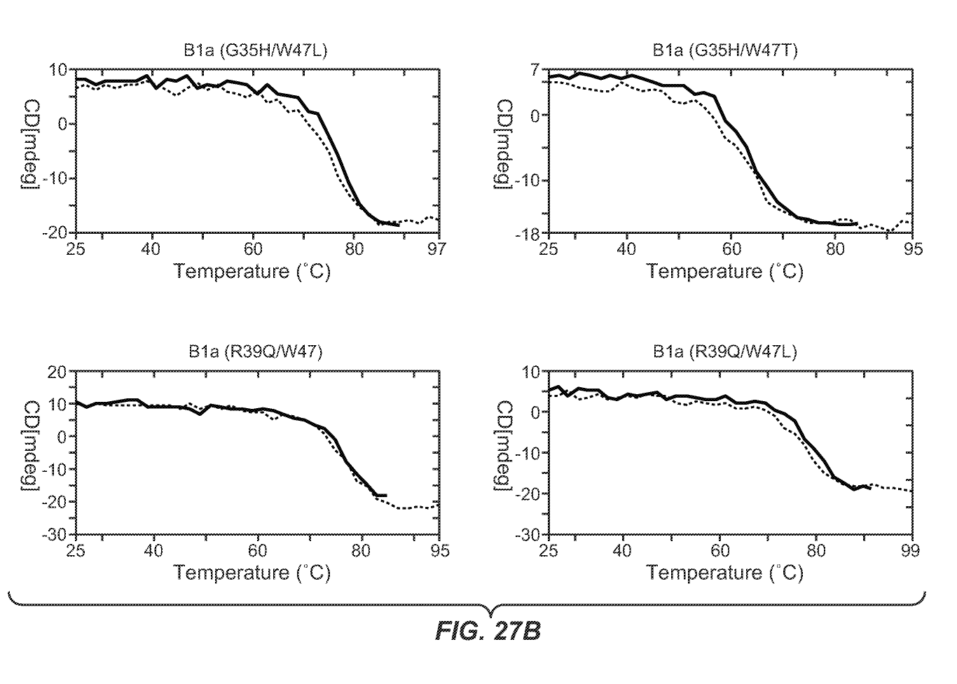

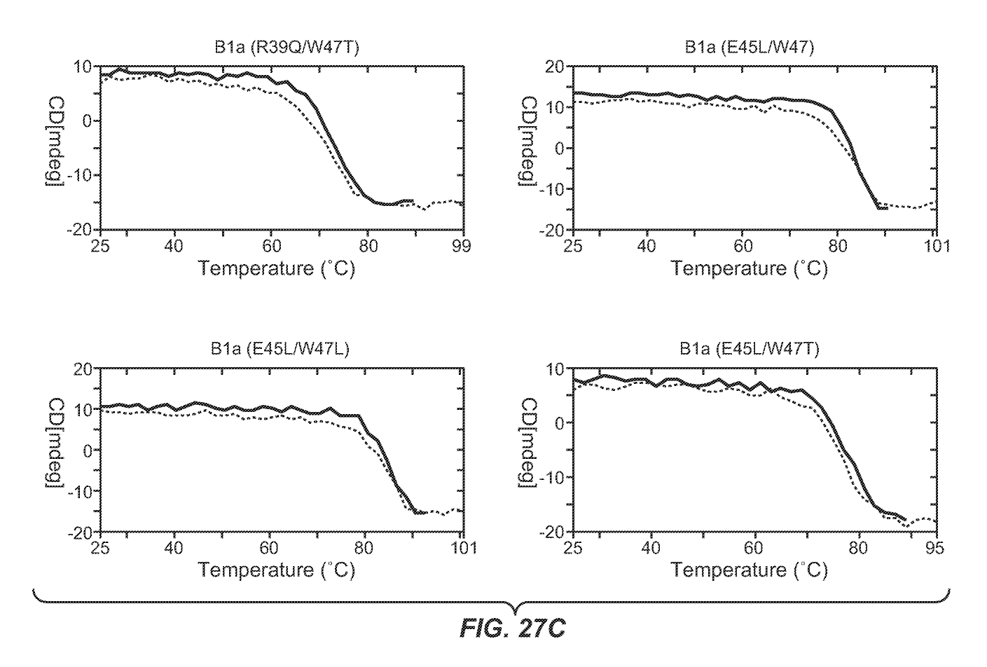

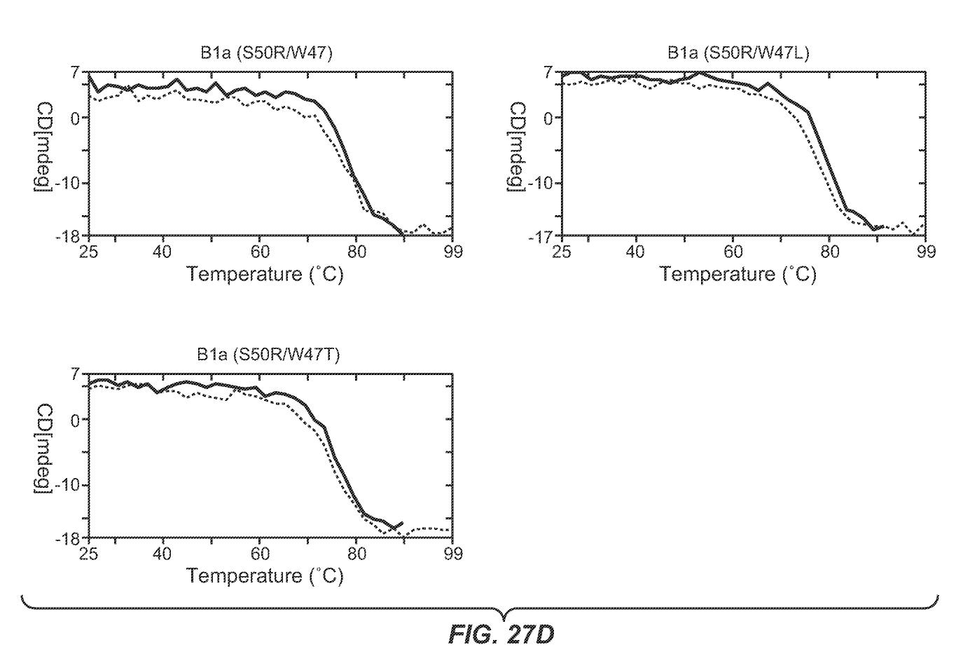

Each mutant was subsequently expressed in 500 mL shake flasks of E. coli BL21 and purified by Protein A chromatography, as described previously. Each clone was analyzed using three different criteria: the purification yield after protein A purification (using the protocols described in Example 1(D)(1); data from the results are shown in FIGS. 24A-24B), the oligomeric state as determined by gel filtration analysis (using the protocols described in Example 1(D)(2); the results are shown in FIGS. 25A-25F)), and the thermal stability and folding percentage, as determined by circular dichroism (using the protocols described in Example 1 (D)(3); the results are shown graphically in FIGS. 26A-26H and 27A-27D, and in tabular form in FIGS. 24A and 24B). FIGS. 24-27 contain graphs and data that are referred to in the following descriptions of the B1a VH domain and B1a mutant VH domains.

Each of the mutated B1a proteins were expressed in E. coli as a soluble protein and the resulting cell lysates were purified by chromatography on columns containing Protein A-coupled resin using the procedures described in Example 1(D)(1). The wild-type B1a protein was Protein A-purified at a yield of up to 7 mg/mL. This protein was 88% monomeric and eluted from the S75 chromatography column after 45 minutes, indicating that it was largely retained on the column. When the glycine at position 35 of the B1a VH domain was mutated back to histidine, the protein could be purified at higher yield (up to 11 mg/L of culture), while the elution time on the gel filtration column remained unchanged. However, the G35H B1a mutant had a clear tendency to aggregate based on its gel filtration column profile (only 57% of the domain had the apparent Mw of a monomer). This showed that a glycine at position 35 was potentially important, possibly to accommodate a bulky residue such as tryptophan at position 47. That tryptophan is physically close to position 35, and although the crystal structure of the B1a VH domain suggests that the tryptophan at position 47 does not fit deeply into the cavity created by the removal of the histidine side chain (unlike the deep fit observed in the case of Hel4 (Jespers et al., supra), even the slight association between the cleft at position 35 and W47 seems to stabilize the protein. If W47 were solvent-exposed in both the B1a VH domain and in the B1a(G35H) VH domain, it would explain the fact that the retention time for the two different proteins is the same. An interaction between H35 and W47 is apparently detrimental for the conformational stability of the domain, hypothetically inducing β-sheet deformation. The circular dichroism profiles of the B1a protein (having a glycine at position 35) and B1a (H35) were similar, with both proteins having high melting temperature (80° C.) and still being refoldable after thermodenaturation. The histidine substitution thus did not apparently affect the thermal stability of the domain, although it did affect the propensity of the domain to aggregate. Thus it is apparent that aggregation and thermal stability seem to be influenced by different residues and are not necessarily inter-dependent.

Clone B1a(W47L) had a greatly reduced retention time on the column (31 minutes) and a slightly higher monomeric content (91%) as compared to B1a. This lowered retention time may be attributed to the replacement of the bulky solvent-exposed tryptophan at position 47 with leucine. When the glycine at position 35 was mutated back to histidine in the W47L background, the yield of the mutant B1a protein increased (up to 10 mg/L culture as compared to 6 mg/L for the parental clone), while the retention time remained constant. The monomeric content decreased to 79%, slightly less than that observed upon G35H mutation in the W47 background. That suggests that the aggregation caused by the presence of a histidine side chain at position 35 is somehow reduced when a smaller, aliphatic side chain such as leucine is present at position 47, even though the monomeric ratio still drops quite significantly. The thermal stability was not affected by the presence of a histidine at position 35 in the L47 context, similar to the findings with the G35H mutant in the W47 context.

Clone B1a(W47T) had a similar chromatographic profile to B1a(W47L), a threonine at position 47 apparently also being able to decrease the ‘stickiness’ of the isolated VH domain on the gel filtration matrix. When a histidine was introduced at position 35 in the context of W47T, the chromatographic profiles were similar to that of the G35/W47T mutant. However, the thermal stability of the domain was affected by the presence of the threonine at position 47, either with a Glycine or Histidine at position 35. The melting temperature dropped from 82° C. to 75° C. when L47 was replaced by a threonine in a G35 background, and even more dramatically from 77° C. to 65° C. in a H35 context. Yet, the domain was still able to refold reversibly after thermodenaturation.

Thus, replacement of a histidine with glycine at position 35 was notably beneficial in preventing aggregation and maintaining the monomeric form of the B1a VH domain in solution, particularly when there was also a tryptophan residue at position 47. However, replacement of a histidine with a glycine at position 35 had no beneficial effect on the thermal stability of the domain or its retention time. Moreover, removal of a bulky sidechain at position 47 greatly reduced the retention time of the molecule and had an effect on its propensity to aggregate.

Introduction of an arginine at position 39 in the B1a VH domain had no beneficial effect on either the protein yield or the retention time of the molecule. Indeed, mutating this position back to its original amino acid, a glutamine, did not affect the protein yield or retention time of the domain in any of the three position 47 backgrounds analyzed in the study. Nevertheless, introduction of an arginine at position 39 significantly reduced the aggregation tendency of the VH domain, especially in the W47 framework context (increasing the monomer percentage from 79% to 88% with W47, from 85% to 91% with L47, and from 88% to 90% with T47). The presence of an arginine residue at position 39 also enhanced the thermal stability of the domain in all backgrounds (an observed decrease in melting temperature from 75° C. to 80° C. with W47, from 75° C. to 82° C. with L47, and from 70° C. to 75° C. with T47). The refoldability of the domain was not affected in any of the mutants made. Finally, as already discussed in the context of the G35H and G35 studies, the presence of a threonine residue at position 47 affects the melting temperature of the VH domain.

Introduction of a leucine residue in place of a glutamate residue at position 45 slightly increased the protein yield when a tryptophan or leucine was present at position 47. More importantly, the retention time of the VH domain was considerably reduced in the E45L/W47 mutant as compared to the E45/W47 molecule—from 75 minutes to 45 minutes. The retention time was also reduced to a lesser extent in the presence of leucine at position 47 (from 37 minutes to 33 minutes). The retention time of B1a(E45L/W47T) was similar to that of B1a (W47T), suggesting that perhaps the presence of a hydrophilic residue such as threonine at position 47 can compensate for the absence of hydrophilic residue like glutamate at position 45. The presence of a glutamate residue at position 45 also apparently reduced the aggregation tendency of the VH domain (increasing the monomer percentage from 80% to 88% in the W47 context, from 87% to 91% in the L47 context, and from 79% to 90% in the T47 context). However, glutamate was slightly unfavorable for the thermal stability of the domain in the presence of tryptophan or leucine at position 47 (an observed decrease in melting temperature from 85° C. to 80° C. with W47 and from 85° C. to 82° C. with L47). The refoldability of the domain was not affected in any case. Finally, as observed previously with glycine or histidine at position 35 and with arginine or glutamate at position 39, the presence of a threonine at position 47 affects the melting temperature of the VH domain (from 85° C. in clones B1a(E45L/W47) or B1a(E45L/W47L) to 75° C. in clone B1a(E45L/W47T).

The serine at position 50 of the B1a VH domain was disadvantageous in many aspects (retention time, aggregation tendency, and protein yield). Substitution of the serine at that position with an arginine residue dramatically reduced the retention time of the domain when a tryptophan was present at position 47 (from 45 minutes to 30 minutes). This same S50R substitution decreased retention time to a lesser extent when a leucine was present at position 47 (from 31 minutes to 29 minutes). The same beneficial effect of the S50R B1a mutation was observed for aggregation (increase in monomer percentage from 88% to 92% with W47, from 91% to 96% with L47, and from 90% to 96% with T47). The protein yield was also improved in all of the position 47 contexts studied (increase in yield from 7 mg/L to 9 mg/L for W47, from 6 mg/L to 7 mg/L with L47, and from 6 mg/L to 8 mg/L with T47). The melting temperature was the only parameter negatively affected by an S50R mutation in the B1a VH domain, and only in the context of W47 and W47L (decrease in melting temperature from 80° C. to 75° C. with W47 and from 82° C. to 75° C. with L47). In the R50 background, threonine has no negative effect on the melting temperature. Structurally, positions 35, 47, and 50 are in very close contact. Serine is a hydrophilic residue but is not charged at physiological/neutral pH. Arginine, though, is positively charged at physiological pH. While not being bound by any particular theory, it is possible that a positive charge at position 50 may interact favorably with neighboring residues in the structure, such as positions 35 and 47, and/or that a positive charge at position 50 stabilizes the domain through the formation of a salt bridge with a negatively charged residue such as a glutamic acid at position 45.

### Example 8

### Effects of Combining Several Mutants on Stability and Folding

The preceding mutagenesis studies highlighted the importance of several residues in the VH domain for stability and proper folding of that domain. To assess whether combinations of modifications at such residues might further enhance the stability/folding of the domain, a number of VH domains including multiple mutations were constructed. Eight mutant B1a VH domains were constructed using Kunkel mutagenesis on a VH domain already containing a W47L mutation as described above: B1a (W47L/W103R); B1a(W47L/V37S/S50R); B1a(W47L/V37S/W103S); B1a(W47L/V37S/W103R); B1a(W47L/S50R/W103S); B1a(W47L/S50R/W103R); B1a(W47L/V37S/S50R/W103S); and B1a(W47L/V37S/S50R/W103R). The oligonucleotides used in the mutagenesis were as follows:

(SEQ ID NO: 265)

G46 (V37S mutation)
GAC ACC TAT ATA GGA TGG TCT CGT CGG GCC CCG GGT

(SEQ ID NO: 266)

G47 (S50R mutation)
GAG GAA CTG GTT GCA CGT ATT TAT CCT ACG AAT GGT

(SEQ ID NO: 267)

G48 (W103S mutation)
TTC TAT GCT ATG GAC TAC TCT GGT CAA GGA ACA CTA
GTC

(SEQ ID NO: 268)

G49 (W103R mutation)
TTC TAT GCT ATG GAC TAC CGT GGT CAA GGA ACA CTA
GTC

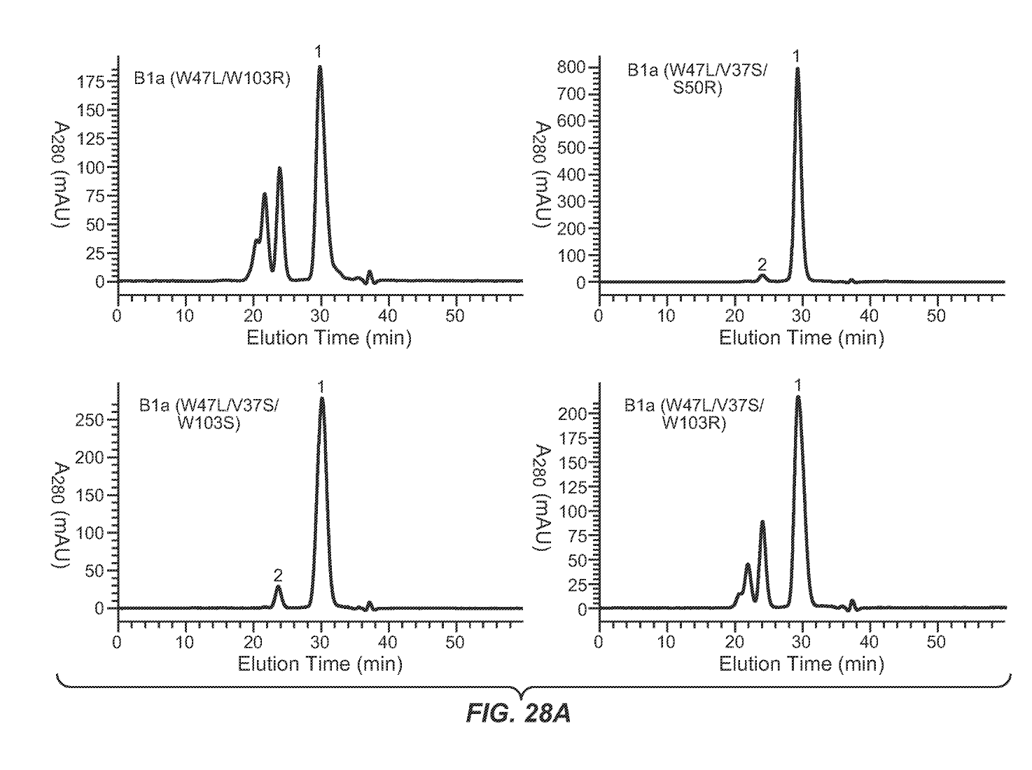

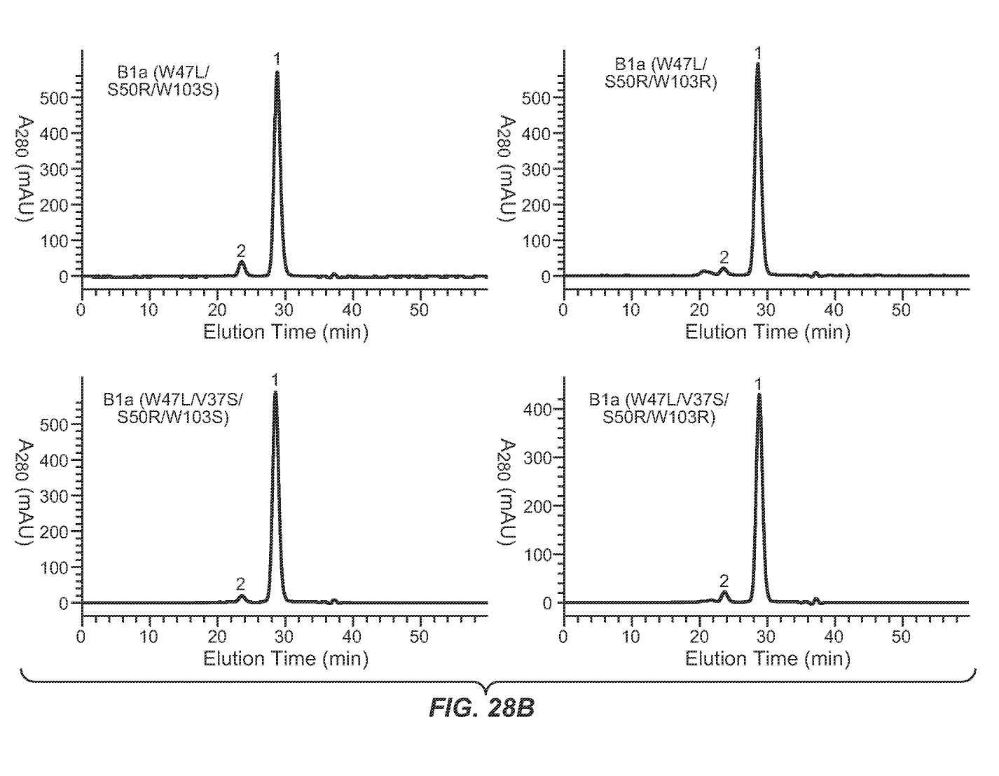

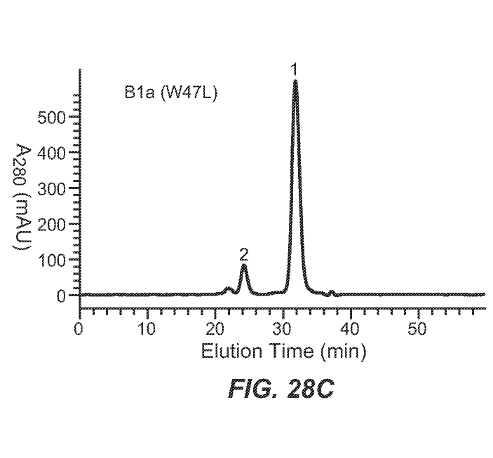

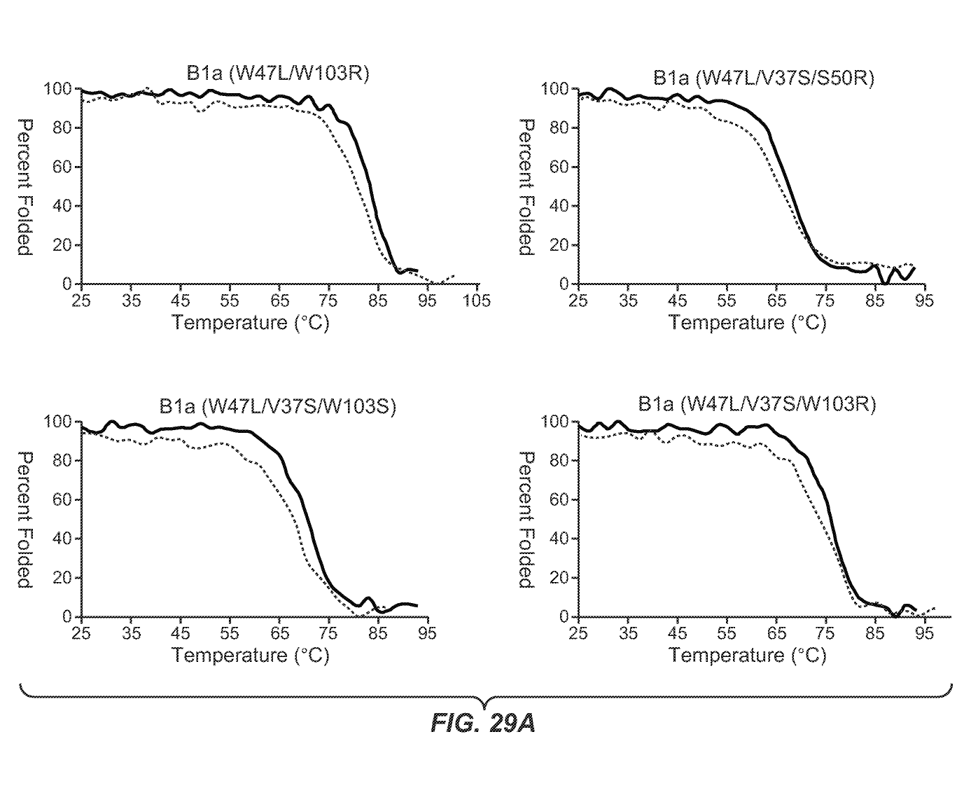

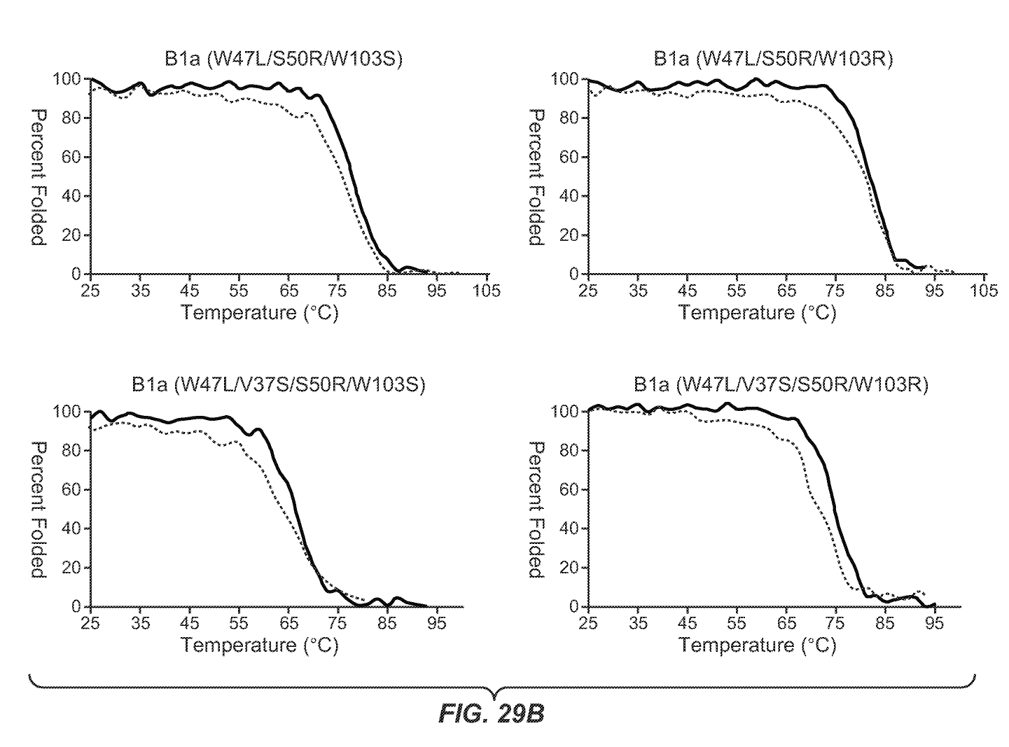

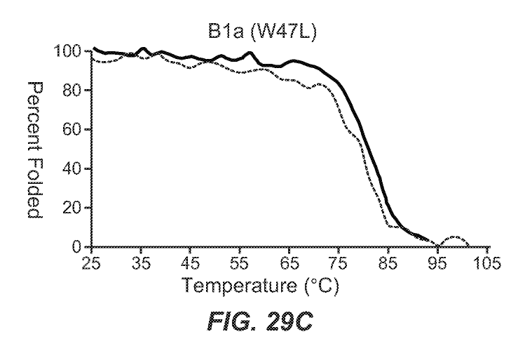

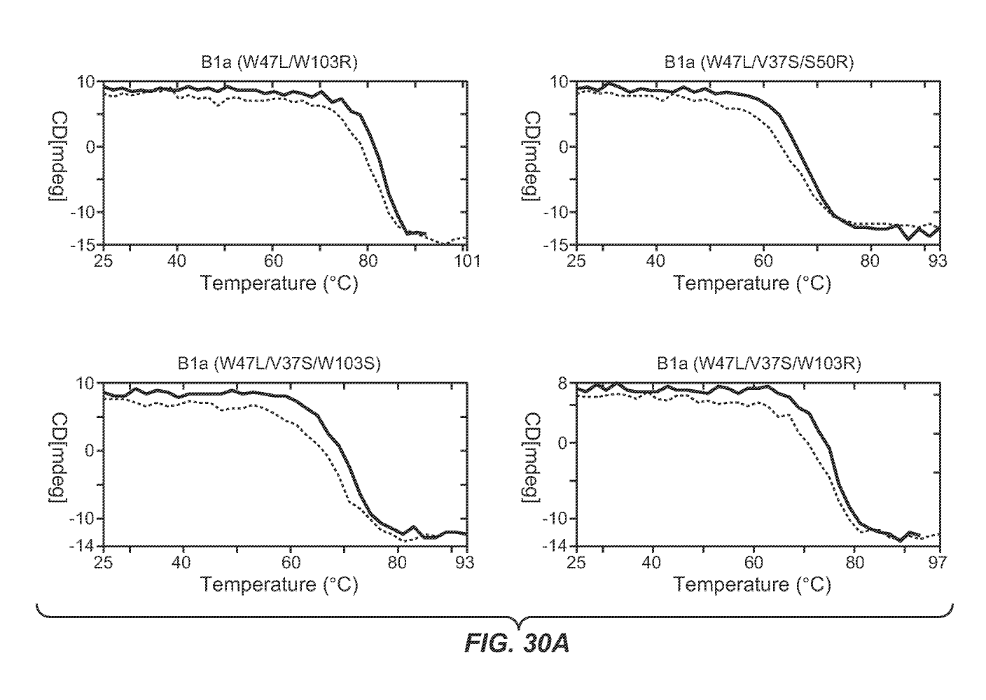

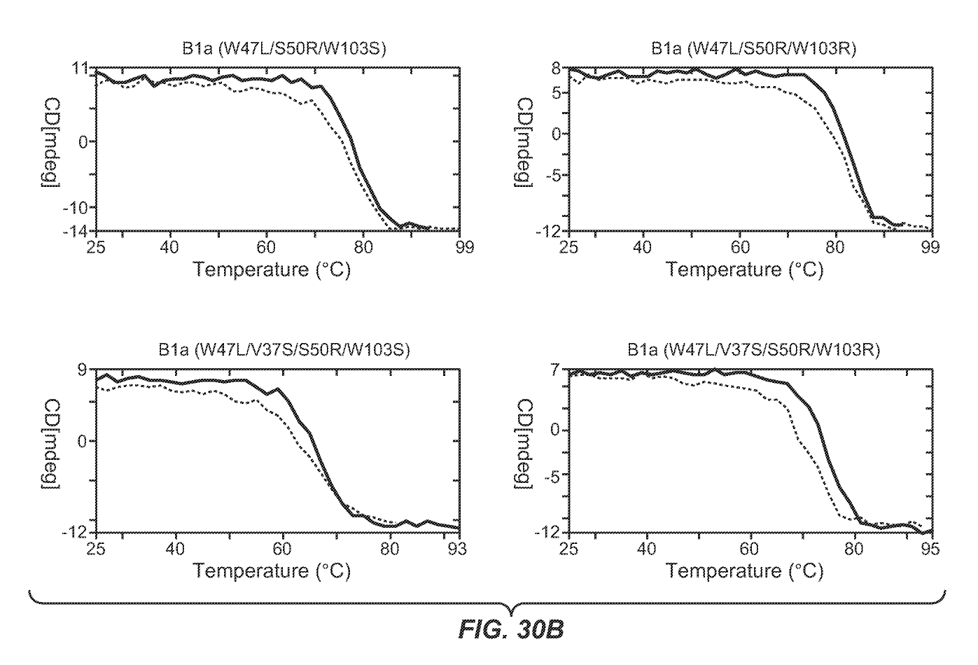

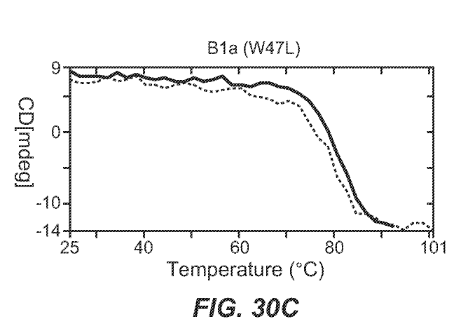

Each mutant was subsequently expressed in 500 mL shake flasks of E. coli BL21 and purified by Protein A chromatography, as described previously. Each clone was analyzed using three different criteria: the purification yield after protein A purification (using the protocols described in Example 1(D)(1); data from the results are shown in FIGS. 24A and 24B), the oligomeric state as determined by light scattering analysis (using the protocols described in Example 1(D)(2); the results are shown in FIGS. 28A-28C and FIGS. 24A and 24B)), and the thermal stability and folding percentage, as determined by circular dichroism (using the protocols described in Example 1(D)(3); the results are shown graphically in FIGS. 29A-29C and 30A-30C, and in tabular form in FIGS. 24A and 24B). FIGS. 24A, 24B, and 28-30 contain graphs and data that are referred to in the following descriptions of the B1a VH domain and B1a mutant VH domains.

Each of the mutated B1a proteins were expressed in E. coli as a soluble protein and the resulting cell lysates were purified by chromatography on columns containing Protein A-coupled resin using the procedures described in Example 1(D)(1). The B1a(W47L) protein was Protein A-purified at a yield of up to 6 mg/mL (see Example 7). This protein was 91.5% monomeric, had a Tₘ of 82° C. and eluted from the S75 chromatography column after 32 minutes, indicating that it was retained on the column. As shown in Example 7, Clone B1a(W47L/V37S) had increased monomeric content (about 97%) over B1a(W47L), but significantly decreased thermal stability (Tₘ of 72° C. versus the B1a(W47L) Tₘ of 82° C.) (see FIGS. 24A and 24B). Also shown in Example 7, Clone B1a(W47L/S50R) had a greater yield and greater monomeric percentage (about 96%) than the W47L clone, but a decreased Tₘ (77° C., higher than that observed for the W47L/V37S mutant) (see FIGS. 24A and 24B). Those two mutations were thus combined, and the triple mutant was characterized. Clone B1a(W47L/V37S/S50R) displayed a better yield than the W47L/V37S mutant, but lesser than either the W47L mutant or the W47L/S50R mutant. This triple mutant also had a high (97%) monomeric content, similar to the W47L/V37S mutant, but higher than either the W47L or W47L/S50R mutants. However, the triple mutant had a significantly lower Tₘ than any of these other mutants (66° C.), demonstrating that neither S50R nor V37S can compensate for their separate detrimental effects on the thermal stability of the protein.

The effect of mutations at position 103 (expected to increase hydrophilicity of the former VL interface) was also examined in the context of the W47L mutation. When the tryptophan at position 103 of the B1a VH domain was mutated to arginine, the protein could be purified at higher yield (up to 7 mg/L of culture). However, the W47L/W103R mutant had a clear tendency to aggregate based on its gel filtration column profile (only 56% of the domain had the apparent Mw of a monomer). This showed that an arginine at position 103 was potentially promoting self-aggregation of the VH domain. The circular dichroism profiles of the B1a(W47L) protein and B1a(W47L/W103R) show that the W103R mutation slightly increased the thermal stability of the domain. The W47L/W103S clone (described in Example 7) had a lesser yield than the W47L/W103R clone, but a much higher monomer percentage. W103S does not appear to affect the monomeric content of the protein or its thermal stability but removes a bulky hydrophobic residue from the former VL interface and reduces the propensity of the domain to interact with the gel filtration matrix, as shown by a reduction in the retention time on a gel filtration column.

Clone B1a(W47L/V37S/W103R) had a lower yield and Tₘ than either the W47L or the W47L/W103R mutants, but did have an improved monomer percentage (69%) over B1a(W47L/W103R). When the tryptophan at position 103 was replaced by serine rather than arginine (B1a(W47L/V37S/W103S)), the yield improved over the W103R mutant, but was still less than the yields obtained for the W47L or W47L/W103R mutants, or for the W47L/W103S mutant. Even less aggregation was observed in the W47L/V37S/W103S mutant than in the W47L/W103 S mutant, but the Tₘ was significantly lower than that of the W47L/W103R mutant. Clone B1a(W47L/S50R/W103S) had a lower yield, but a higher percentage of monomer content than either the W47L or the W47L/S50R mutants, and a lower Tₘ than the W47L mutant. Clone B1a(W47L/S50R/W103R) had the same yield as the W47L mutant, but a lower yield than the W47L/S50R mutant, a higher percentage of monomer content than either of the other two mutants (and significantly higher than the W47L/S50R mutant), and a slightly lower Tₘ than either of the other two mutants. The inclusion of mutations at position 103 in the context of W47L and either V37S or S50R thus generally tended to decrease aggregation but at the expense of thermal stability.

The combined effects of mutations at all four positions (47, 37, 50, and 103) were assessed. The clone B1a(W47L/V37S/S50R/W103S) had a similar or better yield than either the V37S or S50R triple mutant containing W103S, and a higher monomer percentage (97%) than either triple mutant, but a significantly lower Tₘ than either triple mutant (66° C.). The clone B1a(W37L/V37S/S50R/W103R) had a better yield than the W47L/V37S/W103R triple mutant but a lesser yield than the W47L/S50R/W103R triple mutant. The monomer percentage was identical to that of the S50R triple mutant, but greater than that of the V37S triple mutant. The Tₘ, however, was less than either of the triple mutants.

The yield of each of the above-described mutants was reduced compared to the parental clone B1a(W47L). The best combination appears to be B1a(W47L/S50R/W103R). However, other mutants have showed the individual contribution of W103R to the aggregation of the domain. Therefore even though S50R seems to compensate for the negative effect of W103R, it may be more productive for synthetic library construction to use B1a(W47L/S50R/W103S).

1. An isolated antibody variable domain wherein the antibody variable domain comprises one or more amino acid alterations as compared to the naturally-occurring antibody variable domain, and wherein the one or more amino acid alterations increase the stability of the antibody variable domain.

2. The antibody variable domain of claim 1, wherein the antibody variable domain is a heavy chain antibody variable domain.

3. The isolated heavy chain antibody variable domain of claim 2, wherein the isolated heavy chain antibody variable domain is of the VH3 subgroup.

4. The isolated heavy chain antibody variable domain of claim 2, wherein the increased stability of the isolated heavy chain antibody variable domain is measured by a decrease in aggregation of the isolated heavy chain antibody variable domain.

5. The isolated heavy chain antibody variable domain of claim 2, wherein the increased stability of the isolated heavy chain antibody variable domain is measured by an increase in Tₘ of the isolated heavy chain antibody variable domain.

6. The isolated heavy chain antibody variable domain of claim 2, wherein the one or more amino acid alterations increase the hydrophilicity of a portion of the isolated heavy chain antibody variable domain responsible for interacting with a light chain variable domain.

7. The isolated heavy chain antibody variable domain of claim 2, wherein the one or more amino acid alterations are selected from alterations at amino acid positions 35, 37, 45, 47, and 93-102.

8. The isolated heavy chain antibody variable domain of claim 7, wherein amino acid position 35 is alanine, amino acid position 45 is valine, amino acid position 47 is methionine, amino acid position 93 is threonine, amino acid position 94 is serine, amino acid position 95 is lysine, amino acid position 96 is lysine, amino acid position 97 is lysine, amino acid position 98 is serine, amino acid position 99 is serine, amino acid position 100 is proline, and amino acid position 100a is isoleucine.

9. The isolated heavy chain antibody variable domain of claim 8, wherein the isolated heavy chain antibody variable domain has an amino acid sequence comprising SEQ ID NOs: 28 and 54.

10. The isolated heavy chain antibody variable domain of claim 9, wherein amino acid position 35 is glycine, amino acid position 45 is tyrosine, amino acid position 93 is arginine, amino acid position 94 is threonine, amino acid position 95 is phenylalanine, amino acid position 96 is threonine, amino acid position 97 is threonine, amino acid position 98 is asparagine, amino acid position 99 is serine, amino acid position 100 is lysine, and amino acid position 100a is lysine.

11. The isolated heavy chain antibody variable domain of claim 10, wherein the isolated heavy chain antibody variable domain has an amino acid sequence comprising SEQ ID NOs: 26 and 52.

12. The isolated heavy chain antibody variable domain of claim 7, wherein amino acid position 35 is serine, amino acid position 37 is alanine, amino acid position 45 is methionine, amino acid position 47 is serine, amino acid position 93 is valine, amino acid position 94 is threonine, amino acid position 95 is glycine, amino acid position 96 is asparagine, amino acid position 97 is arginine, amino acid position 98 is threonine, amino acid position 99 is leucine, amino acid position 100 is lysine, and amino acid position 100a is lysine.

13. The isolated heavy chain antibody variable domain of claim 12, wherein the isolated heavy chain antibody variable domain has an amino acid sequence comprising SEQ ID NOs: 31 and 57.

14. The isolated heavy chain antibody variable domain of claim 7, wherein amino acid position 35 is serine, amino acid position 45 is arginine, amino acid position 47 is glutamic acid, amino acid position 93 is isoleucine, amino acid position 95 is lysine, amino acid position 96 is leucine, amino acid position 97 is threonine, amino acid position 98 is asparagine, amino acid position 99 is arginine, amino acid position 100 is serine, and amino acid position 100a is arginine.

15. The isolated heavy chain antibody variable domain of claim 14, wherein the isolated heavy chain antibody variable domain has an amino acid sequence comprising SEQ ID NOs: 39 and 65.

16. The isolated heavy chain antibody variable domain of claim 6, wherein the amino acid at amino acid position 35 is a small amino acid.

17. The isolated heavy chain antibody variable domain of claim 16, wherein the small amino acid is selected from glycine, alanine, and serine.

18. The isolated heavy chain antibody variable domain of claim 6, wherein the amino acid at amino acid position 37 is a hydrophobic amino acid.

19. The isolated heavy chain antibody variable domain of claim 18, wherein the hydrophobic amino acid is selected from tryptophan, phenylalanine, and tyrosine.

20. The isolated heavy chain antibody variable domain of claim 6, wherein the amino acid at amino acid position 45 is a hydrophobic amino acid.

21. The isolated heavy chain antibody variable domain of claim 20, wherein the hydrophobic amino acid is selected from tryptophan, phenylalanine, and tyrosine.

22. The isolated heavy chain antibody variable domain of claim 6, wherein amino acid position 35 is selected from glycine and alanine and amino acid position 47 is selected from tryptophan and methionine.

23. The isolated heavy chain antibody variable domain of claim 6, wherein amino acid position 35 is serine, and amino acid position 47 is selected from phenylalanine and glutamic acid.

24. The isolated heavy chain antibody variable domain of claim 2, wherein the one or more amino acid alterations are selected from alterations at amino acid positions 35, 37, 39, 44, 45, 47, 50, 91, 93-100b, 103, and 105.

25. The isolated heavy chain antibody variable domain of claim 24, wherein amino acid position 35 is glycine, amino acid position 39 is arginine, amino acid position 45 is glutamic acid, amino acid position 50 is serine, amino acid position 93 is arginine, amino acid position 94 is serine, amino acid position 95 is leucine, amino acid position 96 is threonine, amino acid position 97 is threonine, amino acid position 99 is serine, amino acid position 100 is lysine, amino acid position 100a is threonine, and amino acid position 103 is arginine.

26. The isolated heavy chain antibody variable domain of claim 25, wherein the isolated heavy chain antibody variable domain has an amino acid sequence comprising SEQ ID NOs: 139 and 215.

27. The isolated heavy chain antibody variable domain of claim 6, wherein the amino acid at any of amino acid positions 39, 45, and 50 are hydrophilic amino acids.

28. The isolated heavy chain antibody variable domain of claim 6, wherein each of the amino acids at amino acid positions 39, 45, and 50 are hydrophilic amino acids.

29. The isolated heavy chain antibody variable domain of claim 28, wherein amino acid position 39 is arginine, amino acid position 45 is glutamic acid, and amino acid position 50 is serine.

30. The isolated heavy chain antibody variable domain of claim 22, wherein each of the amino acids at amino acid positions 39, 45, and 50 are hydrophilic amino acids.

31. The isolated heavy chain antibody variable domain of claim 22, wherein amino acid position 39 is arginine, amino acid position 45 is glutamic acid, and amino acid position 50 is serine.

32. The isolated heavy chain antibody variable domain of claim 6, wherein amino acid positions 37, 44, and 91 are wild-type.

33. The isolated heavy chain antibody variable domain of claim 6, wherein the isolated heavy chain antibody variable domain is tolerant to substitution at each amino acid position in CDR-H3.

34. The isolated heavy chain antibody variable domain of claim 33, wherein the isolated heavy chain antibody variable domain has an amino acid sequence comprising SEQ ID NO: 26.

35. The isolated heavy chain antibody variable domain of claim 33, wherein the isolated heavy chain antibody variable domain has an amino acid sequence comprising SEQ ID NO: 139.

36. The isolated heavy chain antibody variable domain of claim 2, wherein the one or more amino acid alterations are selected from alterations at amino acid positions 35, 37, 39, 44, 45, 47, 50, and 91.

37. The isolated heavy chain antibody variable domain of claim 36, wherein the amino acid at amino acid position 35 is selected from glycine, alanine, serine, and glutamic acid; the amino acid at amino acid position 39 is glutamic acid; and the amino acid at amino acid position 50 is selected from glycine and arginine, and wherein the amino acids at amino acid positions 37, 44, 47, and 91 are wild-type.

38. The isolated heavy chain antibody variable domain of claim 36, wherein the amino acid at amino acid position 35 is glycine, the amino acid at amino acid position 37 is a hydrophobic amino acid; the amino acid at amino acid position 39 is arginine; the amino acid at amino acid position 44 is a small amino acid; the amino acid at amino acid position 45 is glutamic acid; the amino acid at amino acid position 47 is selected from leucine, valine, and alanine; the amino acid at amino acid position 50 is selected from serine and arginine; and the amino acid at amino acid position 91 is a hydrophobic amino acid.

39. The isolated heavy chain antibody variable domain of claim 2, having an amino acid sequence comprising SEQ ID NO: 26.

40. The isolated heavy chain antibody variable domain of claim 2, having an amino acid sequence comprising SEQ ID NO: 139.

41. The isolated heavy chain antibody variable domain of claim 40, further comprising an alteration at amino acid position 35.

42. The isolated heavy chain antibody variable domain of claim 41, wherein the amino acid at amino acid position 35 is selected from glycine, serine and aspartic acid.

43. The isolated heavy chain antibody variable domain of claim 40, further comprising an alteration at amino acid position 39.

44. The isolated heavy chain antibody variable domain of claim 43, wherein the amino acid at amino acid position 39 is aspartic acid.

45. The isolated heavy chain antibody variable domain of claim 40, further comprising an alteration at amino acid position 47.

46. The isolated heavy chain antibody variable domain of claim 45, wherein the amino acid at amino acid position 47 is selected from alanine, glutamic acid, leucine, threonine, and valine.

47. The isolated heavy chain antibody variable domain of claim 40, further comprising alterations at amino acid position 47 and another amino acid position.

48. The isolated heavy chain antibody variable domain of claim 47, wherein the amino acid at amino acid position 47 is glutamic acid and the amino acid at amino acid position 35 is serine.

49. The isolated heavy chain antibody variable domain of claim 47, wherein the amino acid at amino acid position 47 is leucine and the amino acid at amino acid position 37 is selected from serine and threonine.

50. The isolated heavy chain antibody variable domain of claim 47, wherein the amino acid at amino acid position 47 is leucine and the amino acid at amino acid position 39 is selected from serine, threonine, lysine, histidine, glutamine, aspartic acid, and glutamic acid.

51. The isolated heavy chain antibody variable domain of claim 47, wherein the amino acid at amino acid position 37 is leucine and the amino acid at amino acid position 45 is selected from serine, threonine, and histidine.

52. The isolated heavy chain antibody variable domain of claim 47, wherein the amino acid at amino acid position 37 is leucine and the amino acid at amino acid position 103 is selected from serine and threonine.

53. The isolated heavy chain antibody variable domain of claim 2, wherein the amino acid at amino acid position 35 is glycine; wherein the amino acid at amino acid position 39 is arginine; wherein the amino acid at amino acid position 45 is glutamic acid; wherein the amino acid at amino acid position 47 is leucine; and wherein the amino acid at amino acid position 50 is serene.

54. The isolated heavy chain antibody variable domain of claim 53, further comprising a serine at amino acid position 37.

55. The isolated heavy chain antibody variable domain of claim 2, wherein the amino acid at amino acid position 35 is glycine; wherein the amino acid at amino acid position 39 is arginine; wherein the amino acid at amino acid position 45 is glutamic acid; wherein the amino acid at amino acid position 47 is leucine; and wherein the amino acid at amino acid position 50 is arginine.

56. The isolated heavy chain antibody variable domain of claim 2, wherein the amino acid at amino acid position 37 is serine; wherein the amino acid at amino acid position 47 is leucine; wherein the amino acid at amino acid position 50 is arginine; and wherein the amino acid at amino acid position 103 is selected from serine and arginine.

57. The isolated heavy chain antibody variable domain of claim 56, wherein the amino acid at amino acid position 103 is serine.

58. The isolated heavy chain antibody variable domain of claim 56, wherein the amino acid at amino acid position 103 is arginine.

59. The isolated heavy chain antibody variable domains of claim 57, further comprising one or more mutations at amino acid positions 35, 39, or 45.

60. The isolated heavy chain antibody variable domain of claim 59, wherein the amino acid at amino acid position 35 is glycine, the amino acid at amino acid position 39 is arginine, and the amino acid at amino acid position 45 is glutamic acid.

61. A polynucleotide encoding any of the isolated heavy chain antibody variable domain of claim 1.

62. A replicable expression vector comprising a polynucleotide of claim 60.

63. A host cell comprising the replicable expression vector of claim 62.

64. A library of vectors of claim 63, wherein the plurality of vectors encode a plurality of antibody variable domains.

65. A composition comprising at least one isolated heavy chain antibody variable domain, wherein the at least one isolated heavy chain antibody variable domain is selected from the antibody variable domain of claim 1.

66. A plurality of isolated heavy chain antibody variable domains, wherein the isolated heavy chain antibody variable domains are selected from the antibody variable domain of claim 1.

67. The plurality of isolated heavy chain antibody variable domains of claim 66, wherein each isolated heavy chain antibody variable domain comprises one or more variant amino acids in at least one complementarity determining region (CDR) selected from CDR-H1, CDR-H2, and CDR-H3.

68. A method of generating a plurality of isolated heavy chain antibody variable domains, comprising altering one or more framework regions of the heavy chain antibody variable domain as compared to the wild-type heavy chain antibody variable domain, wherein the one or more amino acid alterations increases the stability of the isolated heavy chain antibody variable domain.

69. A method of increasing the stability of an isolated heavy chain antibody variable domain, comprising altering one or more framework amino acids of the isolated heavy chain antibody variable domain as compared to the wild-type heavy chain antibody variable domain, wherein the one or more framework amino acid alterations increases the stability of the isolated heavy chain antibody variable domain.

70. The isolated heavy chain antibody variable domain of claim 23, wherein each of the amino acids at amino acid positions 39, 45, and 50 are hydrophilic amino acids.

71. The isolated heavy chain antibody variable domain of claim 23, wherein amino acid position 39 is arginine, amino acid position 45 is glutamic acid, and amino acid position 50 is serine.

72. The isolated heavy chain antibody variable domain of claim 58, further comprising one or more mutations at amino acid positions 35, 39, or 45.

73. The isolated heavy chain antibody variable domain of claim 72, wherein the amino acid at amino acid position 35 is glycine, the amino acid at amino acid position 39 is arginine, and the amino acid at amino acid position 45 is glutamic acid.
